(19) United States
(12) Patent Application Publication
(10) Pub. No.: US 2025/0177550 A1
(43) Pub. Date: Jun. 5, 2025

(54) A SCORING METHOD FOR AN ANTI-FR ALPHA ANTIBODY DRUG CONJUGATE THERAPY

(71) Applicant: AstraZeneca AB, Södertälje (SE)

(72) Inventors: Nicholas DURHAM, Wilmington, DE (US); Jorge ZERON-MEDINA CUAIRAN, Wilmington, DE (US); Ansh KAPIL, Munchen (DE); Gunter SCHMIDT, Munchen (DE); Jixin WANG, Gaithersburg, MD (US)

(21) Appl. No.: 18/845,835

(22) PCT Filed: Mar. 9, 2023
(86) PCT No.: PCT/EP2023/056028

§ 371 Date: Sep. 10, 2024

Related U.S. Application Data
(60) Provisional application No. 63/319,051, filed on Mar. 11, 2022.

(51) Int. Cl. A61K 47/68; A61P 35/00; G01N 33/574

(52) U.S. Cl. CPC A61K 47/6849; A61K 47/68037; A61K 47/6889; A61P 35/00; G01N 33/57492; G01N 33/57496

(57) ABSTRACT

A method for predicting the efficacy of an antibody drug conjugate (ADC) therapy involves determining the mean optical density (OD) of membrane and optionally cytoplasm staining of each cancer cell in a whole slide image. A diagnostic antibody to which a dye is linked targets the same folate receptor alpha protein as does the ADC antibody. Statistical operations are performed on the OD of staining, such as computing the median, absolute deviation, difference in OD of membrane and cytoplasm staining, percentage of cells a minimum OD, or the sum of minimally stained cells plus insufficiently stained cells near stained cells. For example, a zero, high or low ADC dosage is recommended based on whether median OD falls below, between or above two OD thresholds, respectively. The upper and lower OD thresholds are correlated to responses of training patients treated with the ADC and whose stained cancer tissue has been analyzed.

### TECHNICAL FIELD

The present invention relates to a method for determining a score indicative of how a cancer patient will respond to a therapy that uses an anti-FRα antibody-drug conjugate.

### BACKGROUND

Assessing a cancer patient's probability of response to a given treatment is an essential step in determining a cancer patient's treatment regimen. Such an assessment is often based on histological analysis of tissue samples from a cancer patient and involves identifying and classifying cancers using standard grading schemes. Immunohistochemical (IHC) staining can be used to distinguish marker-positive cells that express a particular protein from marker-negative cells that do not express the protein. IHC staining typically involves multiple dyes, which includes one or more dyes connected to protein-specific antibodies and another dye that is a counterstain. A common counterstain is hematoxylin, which labels DNA and thus stains nuclei.

A protein specific stain or biomarker can be used to identify the regions of the tissue (e.g., tumor tissue) of the cancer patient that are likely to exhibit a response to a predetermined therapy. For example, a biomarker that stains epithelial cells can help to identify the suspected tumor regions. Then other protein specific biomarkers are used to characterize the cells within the cancerous tissue. The cells stained by a specific biomarker can be identified and quantified, and subsequently a score indicating the number of positively stained cells and negatively stained cells can be visually estimated by pathologists. This score can then be compared to scores of other cancer patients that have been calculated in the same way. If the response of these other patients to a given cancer treatment is known, the pathologist can predict, based on a comparison of the score calculated for the cancer patient with the scores of the other patients, how likely the cancer patient is to respond to a given treatment. However, visual assessment by pathologists is prone to variability and subjectivity.

One promising cancer treatment involves an antibody-drug conjugate (ADC) having a drug with cytotoxicity conjugated to an antibody, whose antigen is expressed on the surface of cancer cells. The ADC binds to the antigen and undergoes cellular internalization so as to deliver the drug selectively to cancer cells and to accumulate the drug within those cancer cells and kill them.

Folate receptors (FRs) are membrane-bound proteins present on the cell surface and thus may be exploited to develop new ADCs. The FR family includes FRα, FRβ, FRγ and FRδ. FR binds folate molecules and transports them into cells, such that the folate molecules are delivered to the folate cycle to support metabolism of nucleotides. In particular, folate is important for DNA synthesis, methylation and repair (Cheung, et al., Oncotarget. 2016; 7(32):52553-52574).

FRα is a glycosylphosphatidylinositol (GPI)-anchored membrane protein having high affinity to the active form of folate, 5-methyltetrahydrofolate (5-MTF). Previous studies have shown that FRα plays a crucial role in embryogenesis (Kelemen, Int J Cancer. 2006; 119(2):243-250). Folate transport in adults, however, is mainly driven by ubiquitous expression of reduced folate carriers and proton coupled folate transporters (Zhao, et al., Annu Rev Nutr. 2011; 31:177-201). The distribution of FRα expression in adults is usually limited to the apical surfaces of polarized epithelia, such as choroid plexus, kidney, lung, and placenta.

Overexpression of FRα, which is also known as folate binding protein (FBP) and is associated with the gene FOLR1, is frequently observed in tumor cells such as ovarian, lung (e.g., non-small cell lung cancer (NSCLC)) and breast carcinomas (Shi, et al., Drug Des Devel Ther. 2015; 9:4989-4996). In particular, a previous study has found that the level of soluble FRα in the blood of ovarian cancer patients is elevated, supporting the potential application of FRα as a biomarker of early ovarian cancer (Basal, et al., PLoS One. 2009; 4(7):e6292). Pre-clinical ovarian models have also revealed that overexpression of FRα is associated with tumor progression, and the binding of folate to FRα could mediate activation of the pro-oncogene STAT3 (Hansen, et al., Cell Signal. 2015; 27(7):1356-1368).

### SUMMARY

A computer-based method is sought for generating a repeatable and objective score predicting a cancer patient's response to a treatment involving a therapeutic FRα antibody-drug conjugate.

A method for predicting how a cancer patient will respond to a therapy involving an antibody drug conjugate (ADC) involves determining a predicted efficacy score based on the optical density of membrane and, optionally, cytoplasm staining, by a dye linked to a diagnostic antibody. The ADC includes an ADC payload and an ADC antibody that targets a protein on each cancer cell. Both the diagnostic antibody and the ADC antibody target the folate receptor alpha (FRα) protein on cancer cells.

A tissue sample is immunohistochemically stained using a dye linked to the diagnostic antibody that binds to the FRα protein on the cancer cells in the tissue sample. A digital image of the tissue sample is acquired. Image analysis is performed on the digital image to detect the cancer cells using a convolutional neural network. For each cancer cell, the optical density of staining is determined based on the staining intensities of the dye in the membrane and/or cytoplasm of the cancer cell and/or in the membranes and cytoplasms of other cancer cells that are closer than a predefined distance to the cancer cell. A predicted efficacy score is generated that predicts the response of the cancer patient to the ADC therapy based on various statistical operations performed on the optical density of staining. Patients having a score higher than a predetermined threshold are recommended for a therapy involving the ADC.

In one embodiment, a method of predicting a response of a cancer patient to an anti-FRα ADC involves detecting cancer cells and determining the mean optical density (optionally the median of the mean optical density) of membrane staining of all cancer cells in the digital image of the tissue sample from the cancer patient. The ADC includes an ADC payload and an ADC antibody that targets a FRα protein on cancer cells. A tissue sample is immunohistochemically stained using a dye linked to a diagnostic antibody. The diagnostic antibody binds to the FRα protein on the cancer cells in the tissue sample. A digital image of the tissue sample is acquired, and cancer cells are detected in the digital image. For each cancer cell, the mean staining intensity of the dye in the membrane is measured. In one embodiment, the percentage of cancer cells with a more intense staining intensity than a predetermined threshold is measured. In another embodiment, the staining intensity of the dye in the membranes of other cancer cells that are closer than a predefined distance to the cancer cell is also measured. The staining intensity of each membrane is computed based on the average optical density of a brown diaminobenzidine (DAB) signal in pixels of the membrane. The response of the cancer patient to the ADC is predicted based on whether the predicted efficacy score exceeds a predetermined threshold. A therapy involving the ADC may be administered to the cancer patient when the predicted efficacy score exceeds the predetermined threshold.

In an embodiment relating to the median optical density falling above a threshold, a predicted efficacy score is generated to predict a response of a cancer patient to an ADC that includes an ADC payload and an ADC antibody that targets a folate receptor alpha (FRα) protein on cancer cells. A tissue sample of the cancer patient is immunohistochemically stained using a dye linked to a diagnostic antibody that binds to the FRα protein on the cancer cells in the tissue sample. A digital image of the tissue sample is acquired. For each cancer cell, the mean optical density of staining by the dye in the membrane of the cancer cell is determined. Then the median optical density of staining of all cancer cells in the digital image is determined. A predicted efficacy score is generated for the tissue sample based on the median optical density. The predicted efficacy score is positive if the median optical density is equal to or greater than an optical density threshold and negative if the median optical density is less than the optical density threshold. The optical density threshold is correlated to responses of a cohort of training patients treated with the ADC. A therapy involving the ADC is recommended to the cancer patient if the predicted efficacy score is positive. The therapy involving the ADC may be administered to the cancer patient when the predicted efficacy score is positive.

In an embodiment relating to the percentage of cancer cells whose mean optical density of staining falls above a minimum optical density threshold, a predicted efficacy score is generated that predicts the response of a cancer patient to an ADC that includes an ADC payload and an ADC antibody that targets the FRα protein on cancer cells. A tissue sample is immunohistochemically stained using a dye linked to a diagnostic antibody that binds to the protein on the cancer cells in the tissue sample. A digital image of the tissue sample is acquired. Cancer cells in the digital image are detected using image analysis. For each cancer cell, an optical density of staining of the dye in the cell membrane is determined. Each cancer cell is identified as being either (i) optical-density positive if the optical density of the cancer cell is equal to or greater than an optical density threshold, or (ii) optical-density negative if the mean optical density of the cancer cell is less than the optical density threshold. A predicted efficacy score is generated for the tissue sample based on a percentage of cancer cells in the digital image that are optical-density positive. The predicted efficacy score is positive if the percentage of cancer cells that are optical-density positive is equal to or greater than a percentage threshold and negative if the percentage of cancer cells that are optical-density positive is less than the percentage threshold. The optical density threshold and the percentage threshold are correlated to responses of a cohort of training patients treated with the ADC. A therapy involving the ADC is recommended to the cancer patient if the predicted efficacy score is positive. The therapy involving the ADC may be administered to the cancer patient when the predicted efficacy score is positive.

In an embodiment relating to the spatial proximity of variously stained cells, a cancer patient is identified for treatment with an ADC that includes an ADC payload and an ADC antibody that targets the FRα protein on cancer cells. A tissue sample from the cancer patient is immunohistochemically stained using a dye linked to a diagnostic antibody that binds to the FRα protein on the cancer cells in the tissue sample. A digital image of the tissue sample is acquired. Cancer cells are detected in the digital image. For each cancer cell, the mean optical density of staining by the dye in the cell membrane is determined. Each cancer cell is identified as being either (i) optical-density positive if the mean optical density of the cancer cell is equal to or greater than an optical density threshold, or (ii) optical-density negative if the mean optical density of the cancer cell is less than the optical density threshold. A proximity score for the tissue sample is generated that equals a percentage of the cancer cells in the digital image that are either optical-density positive or optical-density negative but within a predefined distance of an optical-density positive cancer cell. The cancer patient is identified as one who will likely benefit from administration of the ADC if the proximity score exceeds a predetermined percentage threshold. A therapy involving the ADC may be administered to the cancer patient when the proximity score exceeds the predetermined percentage threshold.

In an embodiment relating to the median absolute deviation of the optical density of staining of the cancer cells in a whole slide image, a predicted efficacy score is generated that predicts the response of a cancer patient to an ADC that includes an ADC payload and an ADC antibody that targets the FRα protein on cancer cells. A tissue sample is immunohistochemically stained using a dye linked to a diagnostic antibody that binds to the FRα protein on the cancer cells in the tissue sample. A digital image of the tissue sample is acquired. Cancer cells are detected in the digital image. For each cancer cell, the mean optical density of staining by the dye in the membrane of the cancer cell is determined. Then the median optical density of all cancer cells in the digital image is determined. The median absolute deviation of the optical densities of the cancer cells from the median optical density of all cancer cells in the digital image is determined. A predicted efficacy score for the tissue sample is generated based on the median absolute deviation. The predicted efficacy score is positive if the median absolute deviation is equal to or greater than a deviation threshold and negative if the median absolute deviation is less than the deviation threshold. The deviation threshold is correlated to responses of a cohort of training patients treated with the ADC. A therapy involving the ADC is recommended to the cancer patient if the predicted efficacy score is positive. The therapy involving the ADC may be administered to the cancer patient when the predicted efficacy score is positive.

In an embodiment relating to recommending an effective dosage of an ADC based on membrane staining, a recommended dosage is determined for treating a cancer patient with the ADC that includes an ADC payload and an ADC antibody that targets the FRα protein on cancer cells. A tissue sample is immunohistochemically stained using a dye linked to a diagnostic antibody that binds to the FRα protein on the cancer cells in the tissue sample. A digital image of the tissue sample is acquired. Cancer cells in the digital image are detected. For each cancer cell, a mean optical density of staining by the dye in the membrane of the cancer cell is determined. Then the median optical density of all cancer cells in the digital image is determined. The recommended dosage is determined based on whether the median optical density falls below a lower optical density threshold, between the lower optical density threshold and an upper optical density threshold, or above the upper optical density threshold. The recommended dosage is zero if the median optical density falls below the lower optical density threshold, a higher dosage if the median optical density falls between the lower optical density threshold and the upper optical density threshold, and a lower dosage if the median optical density falls above the upper optical density threshold. The lower optical density threshold and the upper optical density threshold are correlated to responses of a cohort of training patients who were treated with the ADC. Using this method, a treatment recommendation involving the ADC and a recommended dosage is made for the cancer patient. The recommended dosage of the therapy involving the ADC may be administered to the cancer patient based on the median optical density of all cancer cells in the digital image.

In an embodiment relating to the difference between the optical densities of membrane staining and cytoplasm staining, a predicted efficacy score is generated that predicts the response of a cancer patient to an ADC that includes an ADC payload and an ADC antibody that targets the FRα protein on cancer cells. A tissue sample is immunohistochemically stained using a dye linked to a diagnostic antibody that binds to the protein on the membranes and in the cytoplasm of cancer cells in the tissue sample. A digital image, such as a whole slide image, of the tissue sample is acquired. Cancer cells in the digital image are detected using image analysis, optionally involving a convolutional neural network. For each cancer cell, the mean optical density of staining by the dye in the membrane of the cancer cell is determined. For each cancer cell, the mean optical density of staining by the dye in the cytoplasm of the cancer cell is determined. The difference between the mean optical density of staining of the membrane and the mean optical density of staining of the cytoplasm is determined for each cancer cell in the digital image. From among all cancer cells in the digital image, the 85% quantile of the difference between the mean optical density of staining of the membrane and the mean optical density of staining of the cytoplasm is identified. A therapy involving the ADC is recommended to the cancer patient if the 85% quantile of the difference exceeds a predetermined difference threshold. The therapy involving the ADC may be administered to the cancer patient when the 85% quantile of the difference exceeds the predetermined difference threshold.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 shows the amino acid sequences (SEQ ID No: 1 through SEQ ID No.: 36) of various CDRs of various constructs of the ADC anti-FRα antibody.

FIG. 2 shows the amino acid sequences (SEQ ID No: 37 through SEQ ID No.: 48) of the heavy chain and light chain variable regions of various constructs of the anti-FRα antibody.

FIG. 3 shows the amino acid sequences (SEQ ID No: 49 through SEQ ID No.: 54) of the heavy chain and light chain, respectively, of three constructs of the anti-FRα antibody.

FIG. 4 shows the amino acid sequences (SEQ ID No: 55 through SEQ ID No.: 60) of the heavy chain and light chain, respectively, of three additional constructs of the anti-FRα antibody.

FIG. 5 shows the amino acid sequences (SEQ ID No: 61 through SEQ ID No.: 108) of various light-chain and heavy-chain variable framework regions of the anti-FRα antibody.

FIG. 6 shows the amino acid sequences (SEQ ID No: 109 through SEQ ID No.: 111) of three constant domain regions of the anti-FRα antibody.

FIG. 7 is a table illustrating three exemplary number systems used to identify sequences to define a complementarity-determining region (CDR).

FIG. 8 shows the amino acid sequences (SEQ ID No: 112 through SEQ ID No.: 113) of the FRα protein.

FIG. 9 is a flowchart of steps by which an analysis system analyzes digital images of stained tissue to predict how a cancer patient will likely respond to an anti-FRα ADC based on the percentage of cancer cells that are either positively stained or not positively stained but near a positively stained cell.

FIG. 10 shows digital images illustrating the image analysis process of step 12 of FIG. 9.

FIG. 11 illustrates image analysis steps in which nucleus objects of cancer cells are detected.

FIG. 12 illustrates image analysis steps in which nucleus objects are used to detect membranes.

FIG. 13 is a screenshot of the results of the image analysis steps in an image analysis software environment.

FIG. 14 shows a sample calculation of a binary spatial proximity score for ten exemplary cells based on cell separation to reflect the uptake of the ADC payload into neighboring cells.

FIG. 15 illustrates the mechanism by which an anti-FRα ADC therapy kills cancer cells.

FIG. 16 shows sample quantitative results of staining intensities from image analysis using gray values of membrane and cytoplasm pixels.

FIG. 17 lists the exemplary quantitative amounts of staining on the membranes and in the cytoplasms of the image of FIG. 16.

FIG. 18 illustrates the calculation of a continuous spatial proximity score for each of the three cells shown in FIG. 17 based on cell separation to reflect the uptake of the ADC payload into neighboring cells.

FIG. 19 shows a plot and bar graph that correlate a binary spatial proximity score to the actual outcomes of patient-derived xenograft (PDX) studies of patients with ovarian, NSCLC, colon and endometrial cancer.

FIG. 20 is a plot showing the binary spatial proximity score by increasing magnitude for 28 PDX models of ovarian, NSCLC and endometrial cancer.

FIG. 21 is a receive operating characteristic (ROC) curve used to determine the optimal cutpoint to stratify responsive and non-responsive PDX models.

FIG. 22 is a bar graph of the binary spatial proximity score ordered in decreasing magnitude for 197 tissue samples from a database of ovarian cancer patients.

FIG. 23 is a flowchart of steps of a method for predicting the efficacy of an anti-FRα ADC based on the percentage of cancer cells in a whole slide image that exhibit at least a minimum amount of staining.

FIG. 24 shows a plot and bar graph that correlate a predicted efficacy score based on percentage of minimally stained cells to the actual outcomes of PDX studies of patients with ovarian, NSCLC, colon and endometrial cancer.

FIG. 25 is a plot showing the predicted efficacy score (based on % minimally stained cells) by increasing magnitude for 28 PDX models from ovarian, NSCLC and endometrial cancer patients.

FIG. 26 is an ROC curve used to determine the optimal cutpoint to stratify responsive and non-responsive PDX models based on the percentage of minimally stained cells.

FIG. 27 is a bar graph of the predicted efficacy score (based on % minimally stained cells) ordered in decreasing magnitude for 197 tissue samples from a database of ovarian cancer patients.

FIG. 28 is a flowchart of steps of a method for predicting the efficacy of an anti-FRα ADC based on the median of the absolute deviation of staining of each cell from the median membrane staining of all cancer cells in the digital image.

FIG. 29 shows a plot and bar graph that correlate a predicted efficacy score based on the median of the absolute deviation of staining intensity to the actual outcomes of PDX studies of patients with ovarian, NSCLC, colon and endometrial cancer.

FIG. 30 is a plot showing the predicted efficacy score (median absolute deviation of staining) by increasing magnitude for 28 PDX models from ovarian, NSCLC and endometrial cancer patients.

FIG. 31 is an ROC curve used to determine the optimal cutpoint to stratify responsive and non-responsive PDX models based on the median absolute deviation of staining.

FIG. 32 is a bar graph of the predicted efficacy score (median absolute deviation of staining) ordered in decreasing magnitude for 197 tissue samples from a database of ovarian cancer patients.

FIG. 33 is a flowchart of steps of a method for determining the recommended dosage of an anti-FRα ADC based on the median optical density of membrane staining in the digital image.

FIG. 34 shows a plot showing the correlation between the median optical density of membrane staining and tumor response of PDX models from ovarian, NSCLC and endometrial cancer patients to 5 mg/kg of the anti-FRα ADC.

FIG. 35 is a plot showing the predicted efficacy score (based on median optical density of membrane staining) by increasing magnitude for 28 PDX models from ovarian, NSCLC and endometrial cancer patients for 5 mg/kg of the anti-FRα ADC.

FIG. 36 is an ROC curve used to determine the optimal cutpoint to stratify responsive and non-responsive PDX models with 5 mg/kg of the anti-FRα ADC based on the median optical density of membrane staining.

FIG. 37 shows a plot showing the correlation between the median optical density of membrane staining and tumor response of PDX models from ovarian, NSCLC and endometrial cancer patients to 2.5 mg/kg of the anti-FRα ADC.

FIG. 38 is a plot showing the predicted efficacy score (based on median optical density of membrane staining) by increasing magnitude for 28 PDX models from ovarian, NSCLC and endometrial cancer patients for 2.5 mg/kg of the anti-FRα ADC.

FIG. 39 is an ROC curve used to determine the optimal cutpoint to stratify responsive and non-responsive PDX models with 2.5 mg/kg of the anti-FRα ADC based on the median optical density of membrane staining.

FIG. 40 is a graph showing the similar distribution of the FRα protein in ovarian cancer PDX models and in human ovarian tumors.

FIG. 41 is a bar graph of the predicted efficacy score (based on median optical density of membrane staining) ordered in decreasing magnitude for 197 ovarian cancer patients showing upper and lower optical density thresholds that stratify the patients that are to receive higher, lower and zero dosages of the anti-FRα ADC.

FIG. 42 is a flowchart of steps of a method for predicting the efficacy of an anti-FRα ADC based on the difference between the optical densities of membrane staining and cytoplasm staining.

FIG. 43 shows a plot that correlates a predicted efficacy score based on the difference between membrane and cytoplasm staining intensity to the actual outcomes of PDX studies of patients with ovarian, NSCLC, colon and endometrial cancer.

FIG. 44 is a plot showing the predicted efficacy score (difference between membrand and cytoplasm OD) by increasing magnitude for 28 PDX models from ovarian, NSCLC and endometrial cancer patients.

FIG. 45 is an ROC curve used to determine the optimal cutpoint to stratify responsive and non-responsive PDX models based on the difference between membrane and cytoplasm staining intensity.

FIG. 46 is a bar graph of the predicted efficacy score (difference between membrand and cytoplasm OD) ordered in decreasing magnitude for 197 tissue samples from a database of ovarian cancer patients.

### DETAILED DESCRIPTION

The present invention relates to novel methods for predicting a response of a cancer patient to an antibody drug conjugate (ADC) including an ADC antibody that targets the folate receptor alpha (FRα) protein on cancer cells, wherein the response is predicted based on statistical operations using the measured optical density of staining by a dye linked to a diagnostic antibody that also targets the FRα protein. Other aspects of the invention relate to methods of treating a cancer patient by administering a therapy involving the ADC based on a quantitative continuous score (QCS) computed using one of the statistical operations. Yet other aspects of the invention relate to methods of identifying cancer patients for treatment with the ADC based on a QCS score.

A first embodiment involves a novel method for determining a recommended dosage of an FRα antibody-drug conjugate (ADC) for a cancer patient based on whether the median optical density of all cancer cells in a tissue sample stained using a diagnostic antibody that binds to the same protein as does the ADC antibody falls below, between or above two optical density thresholds determined based on the responses of a cohort of training patients treated with the ADC.

A second embodiment relates to a method for generating a predicted efficacy score indicative of how a cancer patient will respond to a therapy involving the ADC based on the median optical density of all cancer cells in a tissue sample of the cancer patient stained using a diagnostic antibody that binds to the same protein as does the ADC antibody.

A third embodiment relates to a method for generating a predicted efficacy score indicative of how a cancer patient will respond to the ADC based on the percentage of cancer cells in the patient's tissue sample that exhibit an optical density of staining that is equal to or greater than an optical density threshold.

A fourth embodiment relates to identifying a cancer patient who will likely benefit from administration of the ADC based on a proximity score equaling a percentage of cancer cells in the patient's tissue sample that either have a mean optical density of staining above an optical density threshold or have a mean optical density of staining below the optical density threshold but are disposed within a predefined distance of a cell whose mean optical density of staining lies above the optical density threshold.

A fifth embodiment relates to a method for generating a predicted efficacy score indicative of how a cancer patient will respond to the ADC based on the median absolute deviation (MAD) of the optical densities of stained cancer cells in a digital image of the patient's tissue sample from the median optical density of all cancer cells in the digital image, wherein the predicted efficacy score predicts a positive response of the patient to the ADC if the predicted efficacy score exceeds a predetermined deviation threshold.

A sixth embodiment relates to a method for generating a predicted efficacy score indicative of how a cancer patient will respond to the ADC based on identifying the 85% quantile of the difference between the optical density of membrane staining and the optical density of cytoplasm staining. The predicted efficacy score predicts a positive response of the patient to the ADC if the predicted efficacy score exceeds a difference threshold.

### I. General Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

Unless otherwise indicated, any nucleic acid sequences are written left to right in 5′ to 3′ orientation. Amino acid sequences are written left to right in amino-to-carboxy orientation, respectively. Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids is defined in conformity with the IUPACIUB Joint Commission on Biochemical Nomenclature (JCBN). A polypeptide may be coded for by more than one nucleotide sequence due to the degeneracy of the genetic code.

As used herein, the term “about” encompasses an acceptable degree of error for the quantity measured given the nature or precision of the measurements. Exemplary degrees of error are within 20 percent (%), typically, within 10%, and more typically, within 5% of a given value or range of values. Preferably, the term “about” as used herein includes plus or minus (±) 5%, preferably ±4%, ±3%, ±2%, ±1%, ±0.5%, ±0.1%, of the numerical value of the number with which it is being used.

Some embodiments of the novel predictive methods disclosed herein involve a Quantitative Continuous Score (QCS). The QCS may indicate a predicted efficacy score, a recommended dosage or an indication of predicted survival time. As used herein, the term “QCS Positive” refers to cancer that is likely to show a response to an anti-FRα ADC therapy. The term “QCS Negative” refers to cancer that is unlikely to show a response to an anti-FRα ADC therapy.

### II. Anti-FRα Antibodies of ADC

### A. Antibody Sequences

The antibodies or antigen-binding fragments of the ADC as defined herein include the listed complementarity-determining region (CDR) sequences or variable heavy and variable light chain sequences (reference antibodies), as well as functional variants thereof. A functional variant binds to the same target antigen as does the reference antibody, and preferably exhibits the same antigen cross-reactivity as does the reference antibody. The functional variants may have a different affinity for the target antigen when compared to the reference antibody, but substantially the same affinity is preferred.

In some embodiments, functional variants of a reference antibody show sequence variation at one or more CDRs when compared to corresponding reference CDR sequences. Thus, a functional antibody variant may include a functional variant of a CDR. Where the term “functional variant” is used in the context of a CDR sequence, this means that the CDR has at most 2, preferably at most 1, amino acid differences when compared to a corresponding reference CDR sequence, and when combined with the remaining 5 CDRs (or variants thereof) enables the variant antibody to bind to the same target antigen as the reference antibody, and preferably to exhibit the same antigen cross-reactivity as the reference antibody. A functional variant may be referred to as a “variant antibody”.

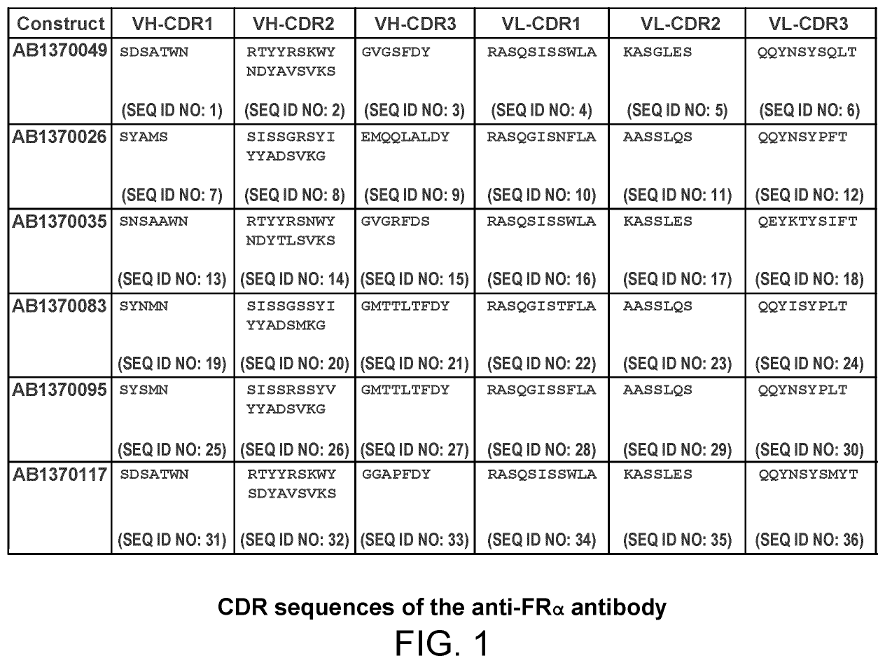

FIGS. 1-6 show the CDR sequences, the VH and VL sequences, the heavy chain and light chain sequences, the FR sequences and the constant domain sequences of the constructs AB1370049, AB1370026, AB1370035, AB1370083, AB1370095 and AB1370117. In the event of any discrepancy between the sequences listed below and the sequences in FIGS. 1-6, the sequences in FIGS. 1-6 take precedence.

FIG. 1 shows various CDRs associated with the ADC antibody. In some embodiments, the ADC antibody or antigen-binding fragment thereof includes CDRs associated with any one of the six constructs AB1370049, AB1370026, AB1370035, AB1370083, AB1370095 or AB1370117, wherein the CDRs are determined by Kabat, Chothia, or IMGT. FIG. 1 lists the sequences SEQ ID NO: 1 through SEQ ID NO: 36 of the CDRs, as defined by Kabat.

In some embodiments, the ADC anti-FRα antibody, or antigen-binding fragment thereof, comprises the following CDRs:
a heavy chain CDR1 of SEQ ID NO: 1 (SDSATWN);
a heavy chain CDR2 of SEQ ID NO: 2 (RTYYRSKWYNDYAVSVKS);
a heavy chain CDR3 of SEQ ID NO: 3 (GVGSFDY);
a light chain CDR1 of SEQ ID NO: 4 (RASQSISSWLA);
a light chain CDR2 of SEQ ID NO: 5 (KASGLES); and
a light chain CDR3 of SEQ ID NO: 6 (QQYNSYSQLT).

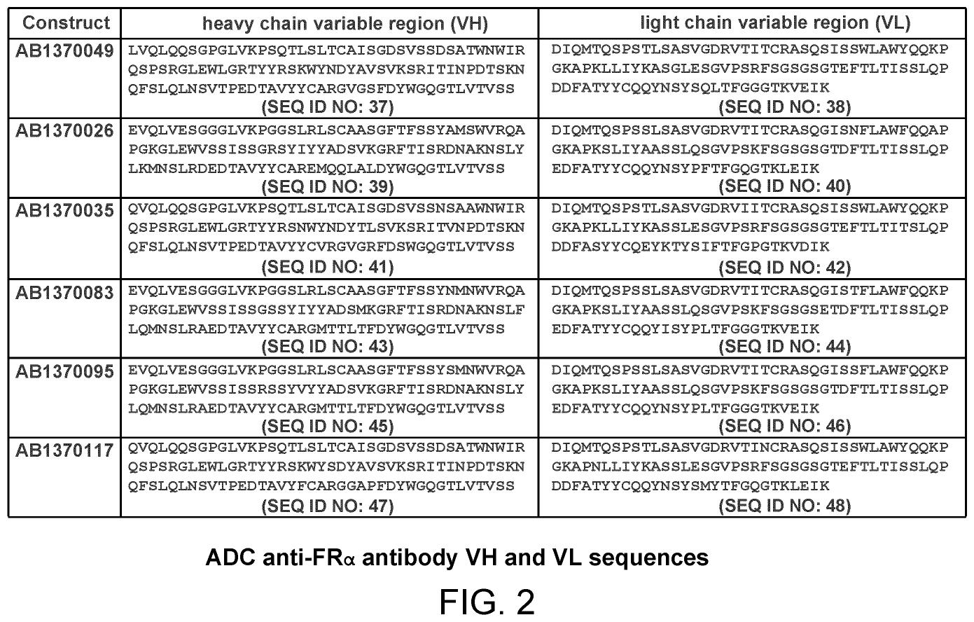

FIG. 2 lists the sequences SEQ ID NO: 37 through SEQ ID NO: 48 of the CDRs.

In some embodiments, the ADC anti-FRα antibody, or antigen-binding fragment thereof, has a heavy-chain variable region (VH) comprising an amino acid sequence that is at least 85% identical, at least 90% identical, at least 95% identical, at least 97% identical, at least 99% identical, or identical to the amino acid sequence of SEQ ID NO: 37 and a light chain variable region (VL) comprising an amino acid sequence that is at least 85% identical, at least 90% identical, at least 95% identical, at least 97% identical, or at least 99% identical, or identical to the amino acid sequence of SEQ ID NO: 38. In some embodiments, the ADC anti-FRα antibody, or antigen-binding fragment thereof, comprises a VH of SEQ ID NO: 37 and a VL of SEQ ID NO: 38.

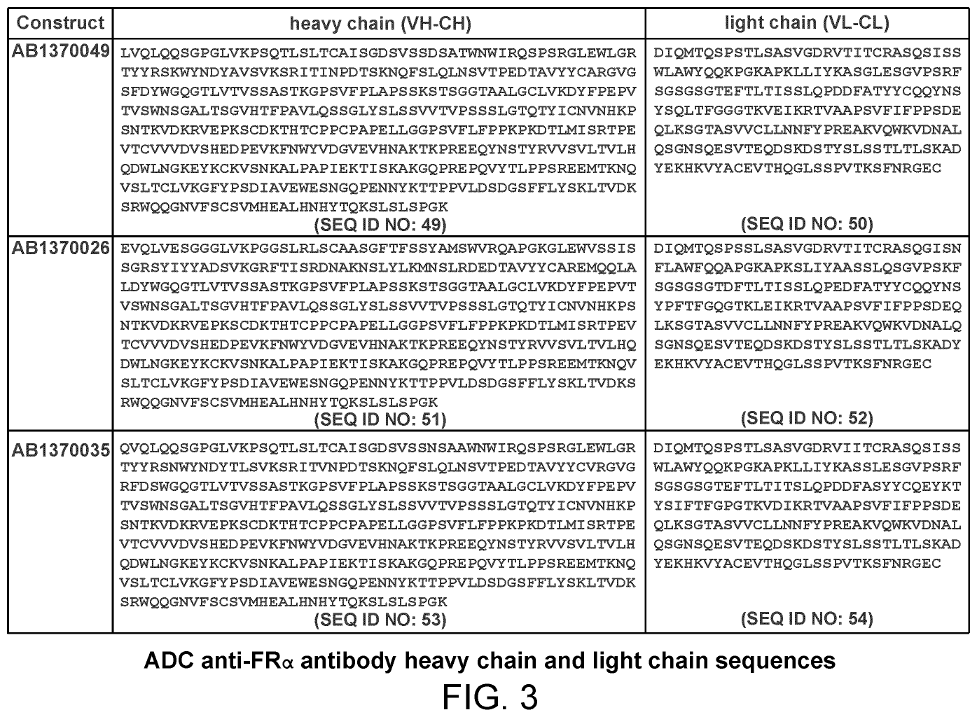

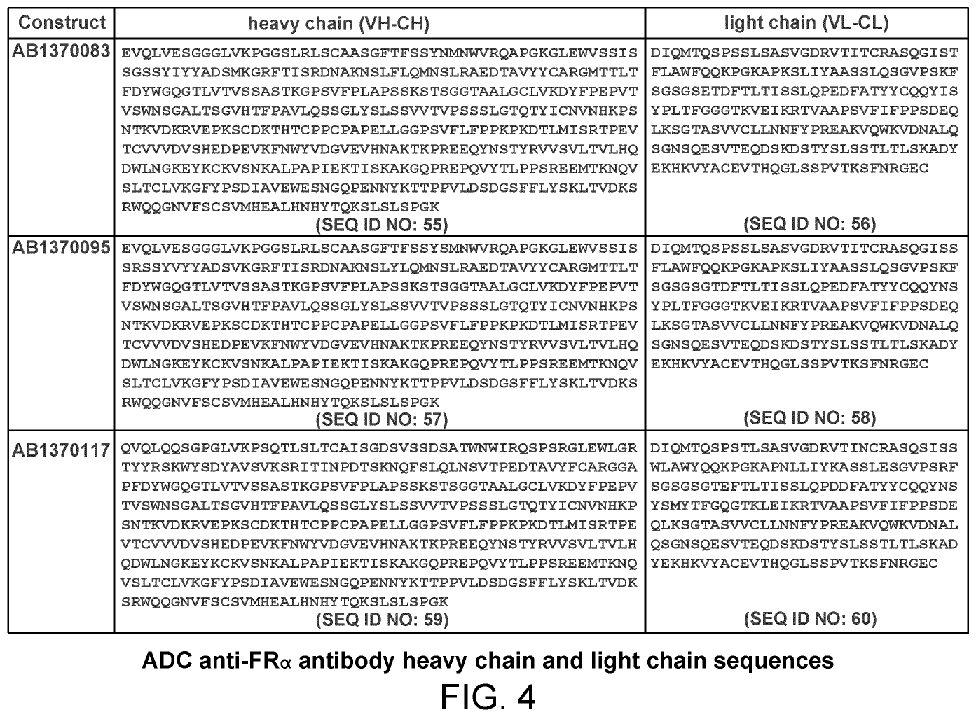

FIG. 3 shows amino acid sequences SEQ ID NO: 49 through SEQ ID NO: 54 of heavy chains (VH-CH) and light chains (VL-CL) of the constructs AB1370049, AB1370026 and AB1370035. FIG. 4 shows amino acid sequences SEQ ID NO: 55 through SEQ ID NO: 60 of VH-CH and VL-CL of the constructs AB1370083, AB1370095 and AB1370117.

In some embodiments, the ADC anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 49 and a light chain comprising an amino acid sequence that is at least 85% identical, at least 90% identical, at least 95% identical, at least 97% identical, at least 99% identical, or identical to the amino acid sequence of SEQ ID NO: 50. In some embodiments, the ADC anti-FRα antibody comprises a heavy chain amino acid sequence of SEQ ID NO: 49 and a light chain amino acid sequence of SEQ ID NO: 50.

In some embodiments, the ADC anti-FRα antibody, or antigen-binding fragment thereof, comprises the following CDRs:
a heavy chain CDR1 of SEQ ID NO: 7 (SYAMS);
a heavy chain CDR2 of SEQ ID NO: 8 (SISSGRSYIYYADSVKG);
a heavy chain CDR3 of SEQ ID NO: 9 (EMQQLALDY);
a light chain CDR1 of SEQ ID NO: 10 (RASQGISNFLA);
a light chain CDR2 of SEQ ID NO: 11 (AASSLQS); and
a light chain CDR3 of SEQ ID NO: 12 (QQYNSYPFT).

In some embodiments, the ADC anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 39 and a VL comprising an amino acid sequence that is at least 85% identical, at least 90% identical, at least 95% identical, at least 97% identical, at least 99% identical, or identical to the amino acid sequence of SEQ ID NO: 40. In some embodiments, the ADC anti-FRα antibody, or antigen-binding fragment thereof, comprises a VH of SEQ ID NO: 39 and a VL of SEQ ID NO: 40.

In some embodiments, the ADC anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 51 and a light chain comprising an amino acid sequence that is at least 85% identical, at least 90% identical, at least 95% identical, at least 97% identical, at least 99% identical, or identical to the amino acid sequence of SEQ ID NO: 52. In some embodiments, the ADC anti-FRα antibody comprises a heavy chain amino acid sequence of SEQ ID NO: 51 and a light chain amino acid sequence of SEQ ID NO: 52.

In some embodiments, the ADC anti-FRα antibody, or antigen-binding fragment thereof, comprises the following CDRs:
a heavy chain CDR1 of SEQ ID NO: 13 (SNSAAWN);
a heavy chain CDR2 of SEQ ID NO: 14 (RTYYRSNWYNDYTLSVKS);
a heavy chain CDR3 of SEQ ID NO: 15 (GVGRFDS);
a light chain CDR1 of SEQ ID NO: 16 (RASQSISSWLA);
a light chain CDR2 of SEQ ID NO: 17 (KASSLES); and
a light chain CDR3 of SEQ ID NO: 18 (QEYKTYSIFT).

In some embodiments, the ADC anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 41 and a VL comprising an amino acid sequence that is at least 85% identical, at least 90% identical, at least 95% identical, at least 97% identical, at least 99% identical, or identical to the amino acid sequence of SEQ ID NO: 42. In some embodiments, the ADC anti-FRα antibody, or antigen-binding fragment thereof, comprises a VH of SEQ ID NO: 41 and a VL of SEQ ID NO: 42.

In some embodiments, the ADC anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 53 and a light chain comprising an amino acid sequence that is at least 85% identical, at least 90% identical, at least 95% identical, at least 97% identical, at least 99% identical, or identical to the amino acid sequence of SEQ ID NO: 54. In some embodiments, the ADC anti-FRα antibody comprises a heavy chain amino acid sequence of SEQ ID NO: 53 and a light chain amino acid sequence of SEQ ID NO: 54.

In some embodiments, the ADC anti-FRα antibody, or antigen-binding fragment thereof, comprises the following CDRs:
a heavy chain CDR1 of SEQ ID NO: 19 (SYNMN);
a heavy chain CDR2 of SEQ ID NO: 20 (SISSGSSYIYYADSMKG);
a heavy chain CDR3 of SEQ ID NO: 21 (GMTTLTFDY);
a light chain CDR1 of SEQ ID NO: 22 (RASQGISTFLA);
a light chain CDR2 of SEQ ID NO: 23 (AASSLQS); and
a light chain CDR3 of SEQ ID NO: 24 (QQYISYPLT).

In some embodiments, the ADC anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 43 and a VL comprising an amino acid sequence that is at least 85% identical, at least 90% identical, at least 95% identical, at least 97% identical, at least 99% identical, or identical to the amino acid sequence of SEQ ID NO: 44. In some embodiments, the ADC anti-FRα antibody, or antigen-binding fragment thereof, comprises a VH of SEQ ID NO: 43 and a VL of SEQ ID NO: 44.

In some embodiments, the ADC anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 55 and a light chain comprising an amino acid sequence that is at least 85% identical, at least 90% identical, at least 95% identical, at least 97% identical, at least 99% identical, or identical to the amino acid sequence of SEQ ID NO: 56. In some embodiments, the ADC anti-FRα antibody comprises a heavy chain amino acid sequence of SEQ ID NO: 55 and a light chain amino acid sequence of SEQ ID NO: 56.

In some embodiments, the ADC anti-FRα antibody, or antigen-binding fragment thereof, comprises the following CDRs:
a heavy chain CDR1 of SEQ ID NO: 25 (SYSMN);
a heavy chain CDR2 of SEQ ID NO: 26 (SISSRSSYVYYADSVKG);
a heavy chain CDR3 of SEQ ID NO: 27 (GMTTLTFDY);
a light chain CDR1 of SEQ ID NO: 28 (RASQGISSFLA);
a light chain CDR2 of SEQ ID NO: 29 (AASSLQS); and
a light chain CDR3 of SEQ ID NO: 30 (QQYNSYPLT).

In some embodiments, the ADC anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 45 and a VL comprising an amino acid sequence that is at least 85% identical, at least 90% identical, at least 95% identical, at least 97% identical, at least 99% identical, or identical to the amino acid sequence of SEQ ID NO: 46. In some embodiments, the ADC anti-FRα antibody, or antigen-binding fragment thereof, comprises a VH of SEQ ID NO: 45 and a VL of SEQ ID NO: 46.

In some embodiments, the ADC anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 57 and a light chain comprising an amino acid sequence that is at least 85% identical, at least 90% identical, at least 95% identical, at least 97% identical, at least 99% identical, or identical to the amino acid sequence of SEQ ID NO: 58. In some embodiments, the ADC anti-FRα antibody comprises a heavy chain amino acid sequence of SEQ ID NO: 57 and a light chain amino acid sequence of SEQ ID NO: 58.

In some embodiments, the ADC anti-FRα antibody, or antigen-binding fragment thereof, comprises the following CDRs:
a heavy chain CDR1 of SEQ ID NO: 31 (SDSATWN);
a heavy chain CDR2 of SEQ ID NO: 32 (RTYYRSKWYSDYAVSVKS);
a heavy chain CDR3 of SEQ ID NO: 33 (GGAPFDY);
a light chain CDR1 of SEQ ID NO: 34 (RASQSISSWLA);
a light chain CDR2 of SEQ ID NO: 35 (KASSLES); and
a light chain CDR3 of SEQ ID NO: 36 (QQYNSYSMYT).

In some embodiments, the ADC anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 47 and a VL comprising an amino acid sequence that is at least 85% identical, at least 90% identical, at least 95% identical, at least 97% identical, at least 99% identical, or identical to the amino acid sequence of SEQ ID NO: 48. In some embodiments, the ADC anti-FRα antibody, or antigen-binding fragment thereof, comprises a VH of SEQ ID NO: 47 and a VL of SEQ ID NO: 48.

In some embodiments, the ADC anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 59 and a light chain comprising an amino acid sequence that is at least 85% identical, at least 90% identical, at least 95% identical, at least 97% identical, at least 99% identical, or identical to the amino acid sequence of SEQ ID NO: 60. In some embodiments, the ADC anti-FRα antibody comprises a heavy chain amino acid sequence of SEQ ID NO: 59 and a light chain amino acid sequence of SEQ ID NO: 60.

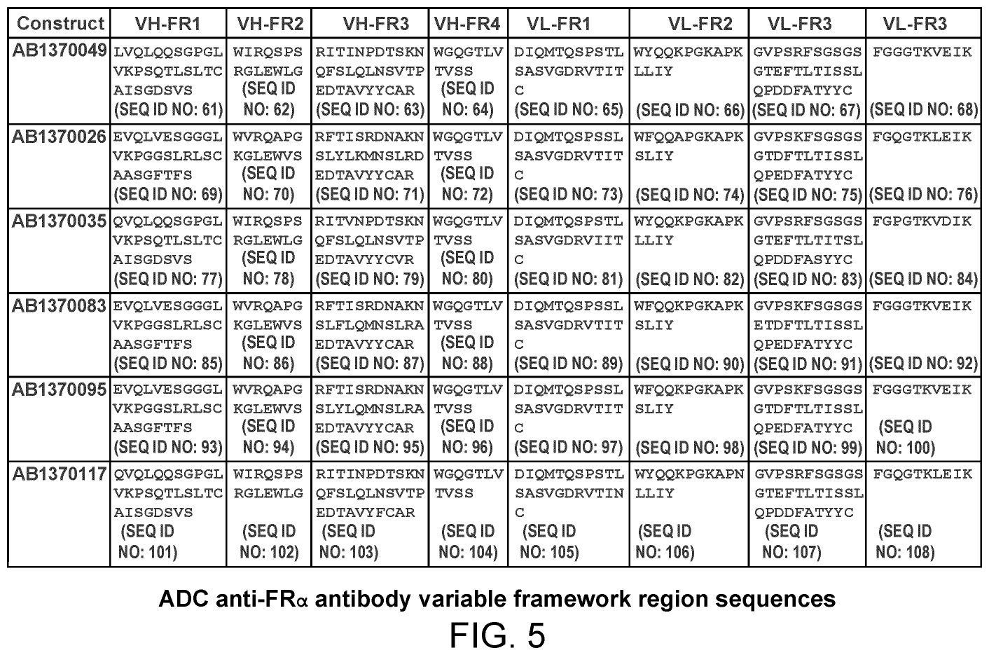

FIG. 5 shows amino acid sequences SEQ ID NO: 61 through SEQ ID NO: 108 of heavy chain and light chain framework regions of the constructs AB1370049, AB1370026, AB1370035, AB1370083, AB1370095 and AB1370117.

In some embodiments, the ADC anti-FRα antibody, or antigen-binding fragment thereof, comprises: (a) light chain VL-FR1, VL-FR2, VL-FR3, and VL-FR4 that are at least 80%, 85%, 90% or 95% identical, or identical to the reference light chains VL-FR1, VL-FR2, VL-FR3, and VL-FR4, respectively, of any one of the constructs AB1370049, AB1370026, AB1370035, AB1370083, AB1370095 or AB1370117; and (b) heavy chain VH-FR1, VH-FR2, VH-FR3, and VH-FR4 that are at least 80%, 85%, 90% or 95% identical, or identical to reference heavy chain VH-FR1, VH-FR2, VH-FR3, and VH-FR4, respectively, of any one of the constructs AB1370049, AB1370026, AB1370035, AB1370083, AB1370095 or AB1370117, as shown in FIG. 5.

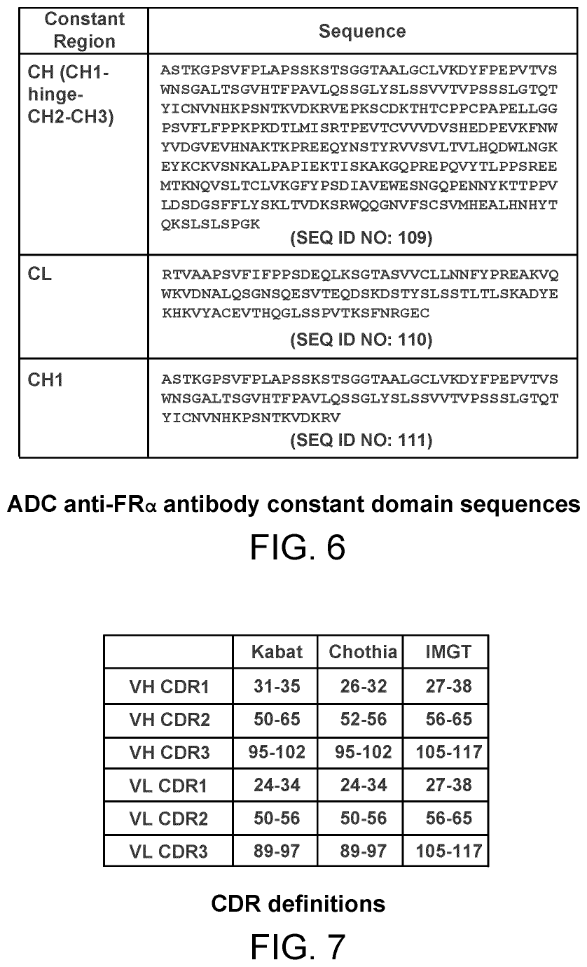

FIG. 6 lists the amino acid sequences SEQ ID NO: 109 through SEQ ID NO: 111 of a heavy chain constant domain, a light chain constant domain, and a heavy chain constant domain CH1.

In some embodiments, the ADC anti-FRα antibody includes a constant heavy chain comprising an amino acid sequence that is at least 85% identical, at least 90% identical, at least 95% identical, at least 97% identical, at least 99% identical, or identical to the amino acid sequence of SEQ ID NO: 109 and a constant light chain comprising an amino acid sequence that is at least 85% identical, at least 90% identical, at least 95% identical, at least 97% identical, at least 99% identical, or identical to the amino acid sequence of SEQ ID NO: 110. In some embodiments, the ADC anti-FRα antibody comprises a constant heavy chain amino acid sequence of SEQ ID NO: 109 and a constant light chain amino acid sequence of SEQ ID NO: 110.

In some embodiments, the ADC anti-FRα antigen-binding fragment comprises a constant heavy chain comprising an amino acid sequence that is at least 85% identical, at least 90% identical, at least 95% identical, at least 97% identical, at least 99% identical, or identical to the amino acid sequence of SEQ ID NO: 111 and a constant light chain comprising an amino acid sequence that is at least 85% identical, at least 90% identical, at least 95% identical, at least 97% identical, at least 99% identical, or identical to the amino acid sequence of SEQ ID NO: 110. In some embodiments, the ADC anti-FRα antigen-binding fragment comprises a constant heavy chain amino acid sequence of SEQ ID NO: 111 and a constant light chain amino acid sequence of SEQ ID NO: 110.

The ADC antibodies may include minor variations in the amino acid sequences, providing that the variations in the amino acid sequences maintain at least 75%, more preferably at least 80%, at least 90%, at least 95%, and most preferably at least 99% sequence identity to the ADC antibody or antigen-binding fragment thereof as defined anywhere herein.

The ADC antibodies may include variants in which amino acid residues from one species are substituted for the corresponding residue in another species, either at the conserved or non-conserved positions. In some embodiments, amino acid residues at non-conserved positions are substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in the ADC antibodies does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

In addition to the twenty standard amino acids, non-standard amino acids (such as 4-hydroxyproline, 6-N-methyl lysine, 2-aminoisobutyric acid, isovaline and α-methyl serine) may be substituted for amino acid residues of the ADC antibodies. A limited number of non-conservative amino acids, amino acids that are not encoded by the genetic code, and unnatural amino acids may be substituted for amino acid residues. The ADC antibodies can also include non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations are carried out in a cell-free system that includes an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). In a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of the ADC antibodies.

Essential amino acids in the ADC antibodies can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g., the translocation or protease components) of the ADC antibodies.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). These authors disclose methods for simultaneously randomizing two or more positions in a polypeptide, selecting for functional polypeptides, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

The “percent sequence identity” between two or more nucleic acid or amino acid sequences is a function of the number of identical positions shared by the sequences. Thus, percent sequence identity may be calculated as the number of identical nucleotides/amino acids divided by the total number of nucleotides/amino acids, multiplied by 100. Calculations of percent sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a person skilled in the art.

Any of a variety of sequence alignment methods can be used to determine percent sequence identity, including, without limitation, global methods, local methods and hybrid methods, such as, e.g., segment approach methods. Protocols to determine percent sequence identity are routine procedures within the scope of one skilled in the art. Global methods align sequences from the beginning to the end of the molecule and determine the best alignment by adding up scores of individual residue pairs and by imposing gap penalties. Non-limiting methods include, for example, CLUSTAL W. See, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement. See, e.g., Osamu Gotoh, Significant Improvement in Accuracy of Multiple Protein Sequence Alignments by Iterative Refinement as Assessed by Reference to Structural Alignments, 264(4) J. MoI. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van WaIIe et al., Align-M: A New Algorithm for Multiple Alignment of Highly Divergent Sequences, 20(9) Bioinformatics: 1428-1435 (2004).

Percent sequence identity can be determined by conventional methods. See, for example, Altschul et al., Bull. Math. Bio. 48:603-16, 1986 and Henikoff and Henikoff, Proc. Natl. Acad. Sci. USA 89:10915-19, 1992. Two amino acid sequences are aligned to optimize the alignment scores using a gap opening penalty of 10, a gap extension penalty of 1, and the “blosum 62” scoring matrix of Henikoff and Henikoff (ibid.).

In some embodiments, the variable domains in both the heavy and light chains of an ADC antibody or antigen-binding fragment thereof are altered by at least partial replacement of one or more CDRs and/or by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It is not necessary to replace all of the CDRs with the complete CDRs from the donor variable region to transfer the antigen-binding capacity of one variable domain to another. Rather, it is only necessary to transfer those residues that are necessary to maintain the activity of the antigen-binding site. Given the explanations set forth in U.S. Pat. Nos. 5,585,089, 5,693,761 and 5,693,762, each of which is incorporated herein by reference, it will be well within the competence of those skilled in the art to carry out routine experimentation to obtain a functional antibody with reduced immunogenicity.

In some embodiments, the ADC antibody or antigen-binding fragment thereof can include, in addition to a VH and a VL, a heavy chain constant region or fragment thereof. In some embodiments, the heavy chain constant region is a human heavy chain constant region, e.g., a human IgG constant region or a human IgG1 constant region.

In some embodiments, a residue is inserted into the heavy chain constant region for site-specific conjugation. For example, a cysteine residue may be inserted between amino acid S239 and V240 in the CH2 region of IgG1, which may be referred to as “a 239 insertion” or “239i.”

In some embodiments, the ADC antibodies disclosed herein can be modified to comprise alterations or modifications to one or more of the three heavy chain constant domains (CH1, CH2 or CH3) and/or to the light chain constant domain (CL). In some embodiments, a modified constant region wherein one or more domains are partially or entirely deleted are contemplated. In some embodiments, a modified ADC antibody will include domain deleted constructs or variants wherein the entire CH2 domain has been removed (ΔCH2 constructs). In some embodiments, the omitted constant region domain can be replaced by a short amino acid spacer (e.g., 10 residues) that provides some of the molecular flexibility typically imparted by the absent constant region. The deletion or inactivation (through point mutations or other means) of a constant region domain can reduce Fc receptor binding of the circulating modified antibody. In other cases it can be that constant region modifications moderate complement binding and thus reduce the serum half-life and nonspecific association of a conjugated ADC payload (e.g., cytotoxin). Yet other modifications of the constant region can be used to eliminate disulfide linkages or oligosaccharide moieties that allow for enhanced localization due to increased antigen specificity or antibody flexibility. In some embodiments, the ADC antibody or antigen-binding fragment thereof has no antibody-dependent cellular cytotoxicity (ADCC) activity and/or no complement-dependent cytotoxicity (CDC) activity.

In some embodiments, the ADC antibody or antigen-binding fragment thereof can be engineered to fuse the CH3 domain directly to the hinge region of the respective modified antibodies or fragments thereof. In other constructs, a peptide spacer can be inserted between the hinge region and the modified CH2 and/or CH3 domains. For example, compatible constructs can be expressed in which the CH2 domain has been deleted, and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. Amino acid spacers can, in some cases, prove to be immunogenic and elicit an unwanted immune response against the construct. In some embodiments, any spacer added to the construct can be relatively non-immunogenic, or even omitted altogether, so as to maintain the desired biochemical qualities of the modified antibodies.

Besides the deletion of whole constant region domains, an ADC antibody or antigen-binding fragment thereof provided herein can be modified by the partial deletion or substitution of a few or even a single amino acid in a constant region. For example, the mutation of a single amino acid in selected areas of the CH2 domain can be enough substantially to reduce Fc binding and thereby increase tumor localization. Similarly, one or more constant region domains that control the effector function (e.g., complement C1Q binding) can be fully or partially deleted. Such partial deletions of the constant regions can improve selected characteristics of the ADC antibody or antigen-binding fragment thereof (e.g., serum half-life) while leaving other desirable functions associated with the subject constant region domain intact. Moreover, the constant regions of the ADC antibody and antigen-binding fragment thereof can be modified through the mutation or substitution of one or more amino acids that enhances the profile of the resulting construct. In this respect it is possible to disrupt the activity provided by a conserved binding site (e.g., Fc binding), while substantially maintaining the configuration and immunogenic profile of the modified antibody or antigen-binding fragment thereof. In some embodiments, there may be an addition of one or more amino acids to the constant region to enhance desirable characteristics such as decreasing or increasing effector function or provide for more ADC payload (e.g., cytotoxin) or carbohydrate attachment. In some embodiments, it can be desirable to insert or replicate specific sequences derived from selected constant region domains.

In some embodiments, a heavy chain constant region or fragment thereof, e.g., a human IgG constant region or fragment thereof, can include one or more amino acid substitutions relative to a wild-type IgG constant domain wherein the modified IgG has an increased half-life compared to the half-life of an IgG having the wild-type IgG constant domain. For example, the IgG constant domain can contain one or more amino acid substitutions of amino acid residues at positions 251-257, 285-290, 308-314, 385-389, and 428-436, wherein the amino acid position numbering is according to the EU index as set forth in Kabat. In some embodiments, the IgG constant domain can contain one or more of a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Serine (S), Histidine (H), Phenylalanine (F), or Tyrosine.

In some embodiments, the ADC antibodies or antigen-binding fragments thereof comprise a YTE mutant. The terms “YTE” or “YTE mutant” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgG1. See U.S. Pat. No. 7,658,921, which is incorporated herein by reference. The YTE mutant has been shown to increase the serum half-life of antibodies approximately four-times as compared to wild-type versions of the same antibody (Dall'Acqua et al., J. Biol. Chem. 281:23514-24 (2006); Robbie et al., (2013) Antimicrob. Agents Chemother. 57, 6147-6153). See also U.S. Pat. No. 7,083,784, which is incorporated herein by reference in its entirety.

In some embodiments, the ADC antibody or antigen-binding fragment thereof comprises:
L at the N-terminus (e.g., position 1) of the VH;
E at the N-terminus (e.g., position 1) of the VH; or
Q at the N-terminus (e.g. position 1) of the VH.

### B. Definitions and Antibody Formats

As used herein, the term “antibody” refers to an immunoglobulin molecule that specifically binds to, or is immunologically reactive with, a particular antigen.

Generally, an antibody includes at least two “light chains” (LC) and two “heavy chains” (HC). The light chains and heavy chains of such antibodies are polypeptides that include several domains. Each heavy chain includes a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain (abbreviated herein as “VL”) and a light chain constant domain (abbreviated herein as “CL”).

In some embodiments, the ADC antibody is a full-length antibody. An “intact” or “full-length” antibody, as used herein, refers to an antibody having two heavy (H) chain polypeptides and two light (L) chain polypeptides interconnected by disulfide bonds.

A “variable region” of an antibody refers to the variable region of the antibody light chain or the variable region of the antibody heavy chain, either alone or in combination. The variable regions VH and VL can be further subdivided into regions of hypervariability, termed complementarity-determining regions (CDRs) (also known as hypervariable regions), interspersed with regions that are more conserved, termed framework regions (FRs). Preferably, each VH and VL is composed of three CDRs and four FRs, arranged from amino-terminus to carboxy-terminus in the following order: FR1, CDR1, FR2, CDR2, FR3, CDR3, FR4. The VH or VL chain of the antibody can further include all or part of a heavy or light chain constant region.

Binding between an antibody and its target antigen or epitope is mediated by the CDRs. The term “epitope” refers to a target protein region (e.g., polypeptide) capable of binding to (e.g., being bound by) an antibody or antigen-binding fragment. The CDRs are the main determinants of antigen specificity. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The sequence of a CDR may be identified by reference to any number system known in the art. FIG. 7 illustrates three exemplary number systems: the Kabat system (Kabat, E. A., et al., Sequences of Proteins of Immunological Interest, 5th ed., Public Health Service, National Institutes of Health, Bethesda, MD (1991); the Chothia system (Chothia &, Lesk, “Canonical Structures for the Hypervariable Regions of Immunoglobulins,” J. Mol. Biol. 196, 901-917 (1987)); and the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)).

The “constant domains” (or “constant regions”) of the heavy chain and of the light chain are not involved directly in binding of an antibody to a target, but instead exhibit various effector functions. The constant regions of the antibodies can mediate the binding of the immunoglobulin to host tissues or factors, including various cells of the immune system (e.g., effector cells) and the first component (Clq) of the classical complement system.

There are five major classes of heavy chain constant region, classified as IgA, IgG, IgD, IgE and IgM, each with characteristic effector functions designated by isotype. Ig molecules interact with multiple classes of cellular receptors. For example, IgG molecules interact with three classes of Fcγ receptors (FcγR) specific for the IgG class of antibody, namely FcγRI, FcγRII, and FcγRIII. Binding of an antibody to Fc receptors on cell surfaces triggers a number of important and diverse biological responses, including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. The important sequences for the binding of IgG to the FcγR receptors have been reported to be located in the CH2 and CH3 domains.

In preferred embodiments, the ADC anti-FRα antibodies or antigen-binding fragments thereof are IgG isotype. The ADC anti-FRα antibodies or antigen-binding fragments can be any IgG subclass, for example IgG1, IgG2, IgG3, or IgG4 isotype. In preferred embodiments, the ADC anti-FRα antibodies or antigen-binding fragments thereof are based on an IgG1 isotype. The use of a wildtype human IgG1 molecule that is close to a natural IgG could reduce developability and other risks.

For heavy chain constant region amino acid positions of the ADC antibodies described herein, numbering is according to the EU index first described in Edelman, G. M., et al., Proc. Natl. Acad. Sci. USA 63 (1969) 78-85). The EU numbering of Edelman is also set forth in Kabat et al. (1991) (supra.). Thus, the terms “EU index as set forth in Kabat”, “EU Index”, “EU index of Kabat” or “EU numbering” in the context of the heavy chain refers to the residue numbering system based on the human IgG1 EU antibody of Edelman et al. as set forth in Kabat et al. (1991). The numbering system used for the light chain constant region amino acid sequence is similarly set forth in Kabat et al. (supra.). Thus, as used herein, “numbered according to Kabat” refers to the Kabat numbering system set forth in Kabat et al. (supra.).

The terms “Fc region”, “Fc part” and “Fc” are used interchangeably herein and refer to the portion of a native immunoglobulin that is formed by two Fc chains. Each “Fc chain” includes a constant domain CH2 and a constant domain CH3. Each Fc chain may also include a hinge region. A native Fc region is homodimeric. In some embodiments, the Fc region may be heterodimeric because it may contain modifications to enforce Fc heterodimerization. The Fc region contains the carbohydrate moiety and binding sites for complement and Fc receptors (including the FcRn receptor), and has no antigen binding activity. Fc can refer to this region in isolation, or this region in the context of an antibody, antibody fragment, or Fc fusion protein. Polymorphisms have been found in a number of Fc domain sites, including but not limited to EU positions 270, 272, 312, 315, 356, and 358, resulting in minor variations between the sequences described in the instant application and sequences known in the art. As a result, every naturally occurring IgG Fc region is referred to as a “wild type IgG Fc domain” or “WT IgG Fc domain” (i.e., any allele). Human IgG1, IgG2, IgG3, and IgG4 heavy chain sequences can be obtained in a variety of sequence databases, including the UniProt database (www.uniprot.org) under accession numbers P01857 (IGHG1_HUMAN), P01859 (IGHG2_HUMAN), P01860 (IGHG3_HUMAN), and P01861 (IGHG4 HUMAN) respectively.

In some embodiments, the ADC anti-FRα antibodies are monoclonal antibodies. A “monoclonal antibody” (mAb) refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” can encompass both full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals. More preferably, the ADC anti-FRα antibodies are isolated monoclonal antibodies. In a more preferable embodiment, the ADC antibody is a fully human monoclonal antibody.

The ADC anti-FRα antibodies and antigen-binding fragments thereof may be derived from any species by recombinant means. For example, the ADC antibodies or antigen-binding fragments may be mouse, rat, goat, horse, swine, bovine, chicken, rabbit, camelid, donkey, human, or chimeric versions thereof. For use in administration to humans, non-human derived ADC antibodies or antigen-binding fragments may be genetically or structurally altered to be less immunogenic upon administration to the human patient. Especially preferred are human or humanized antibodies, especially as recombinant human or humanized antibodies.

The term “human antibody” means an antibody produced in a human or an antibody having an amino acid sequence corresponding to an antibody produced in a human made using any technique known in the art. A human antibody may include intact or full-length antibodies, fragments thereof, and/or antibodies comprising at least one human heavy and/or light chain polypeptide such as, for example, an antibody comprising murine light chain and human heavy chain polypeptides. A human antibody may include amino acid residues not encoded by human germline immunoglobulin sequences (e.g., mutations introduced by random or site-specific mutagenesis in vitro or during gene rearrangement or by somatic mutation in vivo). A human antibody can be made in a human cell (through recombinant expression), a non-human animal, or a prokaryotic or eukaryotic cell that can express functionally rearranged human immunoglobulin (such as heavy and light chain) genes. A linker peptide that is not found in native human antibodies can be included in a single chain human antibody. For example, an Fv may have a linker peptide, such as two to about eight glycine or other amino acid residues, that joins the heavy chain's variable region and the light chain's variable region. These linker peptides are considered to be of human origin. Human antibodies can be produced using a variety of techniques, including phage display techniques that use antibody libraries derived from human immunoglobulin sequences. Transgenic mice that are unable to express functional indigenous immunoglobulins but can express human immunoglobulin genes can also be used to make human antibodies (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598, each of which is incorporated herein by reference). Human antibodies can also be directly prepared using various techniques known in the art. Immortalized human B lymphocytes immunized in vitro or isolated from an immunized individual that produce an antibody directed against a target antigen can be generated. See, e.g., Cole et al., Monoclonal Antibodies and Cancer Therapy, Alan R. Liss, p. 77 (1985); Boemer et al., J. Immunol. 147 (1):86-95 (1991); U.S. Pat. No. 5,750,373.

The term “humanized antibody” refers to antibodies in which the framework or CDRs have been modified to comprise the CDR of an immunoglobulin of different specificity as compared to that of the parent immunoglobulin. For example, a murine CDR may be grafted into the framework region of a human antibody to prepare the “humanized antibody.” See, e.g., Riechmann, L., et al., Nature 332 (1988) 323-327; and Neuberger, M. S., et al., Nature 314 (1985) 268-270. In some embodiments, “humanized antibodies” are those in which the constant region has been additionally modified or changed from that of the original antibody to generate desirable properties.

Humanized antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as FRα. In this way, folate receptor (FR) residues can be selected and combined with the consensus and import sequences so that the desired antibody characteristic, such as increased affinity for the target antigen(s), is achieved.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin, whereas all or substantially all of the FR regions are those of a human immunoglobulin consensus sequence. Humanized antibody can also include at least a portion of an immunoglobulin constant region or domain (Fc), typically that of a human immunoglobulin. Examples of methods used to generate humanized antibodies are described in U.S. Pat. Nos. 5,225,539 and 5,639,641, each of which is incorporated herein by reference.

The term “chimeric antibody” refers to an antibody that includes a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” are those in which the constant region has been modified or changed from that of the original antibody to generate desirable properties. Such chimeric antibodies are also referred to as “class-switched antibodies”. Chimeric antibodies are the product of expressed immunoglobulin genes that include DNA segments encoding immunoglobulin variable regions and DNA segments encoding immunoglobulin constant regions. Methods for producing chimeric antibodies involving conventional recombinant DNA and gene transfection techniques are well known in the art. See, e.g., Morrison, S. L., et al., Proc. Natl. Acad. Sci. USA 81 (1984) 6851-6855; U.S. Pat. Nos. 5,202,238 and 5,204,244, each of which is incorporated herein by reference.

In some embodiments, the ADC antibody is a full-length antibody described above. Alternatively, the ADC antibody can be an antigen-binding fragment. The term “antigen-binding fragment” as used herein includes any naturally-occurring or artificially-constructed configuration of an antigen-binding polypeptide comprising one, two or three light chain CDRs, and/or one, two or three heavy chain CDRs, wherein the polypeptide is capable of binding to the antigen.

In some embodiments, the ADC antigen-binding fragment is a Fab fragment. The ADC antibody can also be a Fab′, an Fv, an scFv, an Fd, a V NAR domain, an IgNAR, an intrabody, an IgG CH2, a minibody, a single-domain antibody, an Fcab, an scFv-Fc, F(ab′)2, a di-scFv, a bi-specific T-cell engager (BiTE®), a F(ab′)3, a tetrabody, a triabody, a diabody, a DVD-Ig, an (scFv)2, a mAb2 or a DARPin.

The terms “Fab fragment” and “Fab” are used interchangeably herein and contain a single light chain (e.g., a constant domain CL and a VL) and a single heavy chain (e.g. a constant domain CH1 and a VH). The heavy chain of a Fab fragment is not capable of forming a disulfide bond with another heavy chain.

A “Fab′ fragment” contains a single light chain and a single heavy chain, but in addition to the CH1 and the VH, a “Fab′ fragment” also contains the region of the heavy chain between the CH1 and CH2 domains that is required for the formation of an inter-chain disulfide bond. Thus, two “Fab′ fragments” can associate via the formation of a disulfide bond to form a F(ab′)2 molecule.

A “F(ab′)2 fragment” contains two light chains and two heavy chains. Each chain includes a portion of the constant region necessary for the formation of an inter-chain disulfide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is antibody fragment containing the VH and VL domain of an antibody, linked together to form a single chain. A polypeptide linker is commonly used to connect the VH and VL domains of the scFv.

A “tandem scFv”, also known as a TandAb®, is a single-chain Fv molecule formed by covalent bonding of two scFvs in a tandem orientation with a flexible peptide linker.

A “bi-specific T cell engager” (BiTE®) is a fusion protein that includes two single-chain variable fragments (scFvs) on a single peptide chain. One of the scFvs binds to T cells via the CD3 receptor, and the other to a tumor cell antigen.

A “diabody” is a small bivalent and bispecific antibody fragment comprising a heavy chain variable domain (VH) connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

A “DARPin” is a bispecific ankyrin repeat molecule. DARPins are derived from natural ankyrin proteins, which can be found in the human genome and are one of the most abundant types of binding proteins. A DARPin library module is defined by natural ankyrin repeat protein sequences, using 229 ankyrin repeats for the initial design and another 2200 for subsequent refinement. The modules serve as building blocks for the DARPin libraries. The library modules resemble human genome sequences. A DARPin is composed of 4 to 6 modules. Because each module is approximately 3.5 kDa, the size of an average DARPin is 16-21 kDa. Selection of binders is done by ribosome display, which is completely cell-free and is described in He M. and Taussig M J., Biochem Soc Trans. 2007, November; 35(Pt 5):962-5.

In some embodiments, the ADC antibody or antigen-binding fragment thereof can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in Remington's Pharmaceutical Sciences, 22nd ed., Ed. Lloyd V. Allen, Jr. (2012).

### C. FRα Binding

In the preferred embodiments, the ADC anti-FRα antibody or antigen-binding fragments thereof, specifically bind to FRα. The term “specifically binding to FRα” refers to an antibody that is capable of binding to the defined target with sufficient affinity such that the antibody is useful as a therapeutic agent in targeting FRα. In some embodiments, the ADC antibody that specifically binds to FRα does not bind to other antigens, or does not bind to other antigens with sufficient affinity to produce a physiological effect. In some embodiments, the ADC anti-FRα antibody or antigen-binding fragments thereof specifically binds to human FRα (UniProt ID: P15328) and/or cynomolgus monkey FRα (UniProt ID: A0A2K5U044). In particularly preferred embodiments, the ADC anti-FRα antibody or antigen-binding fragments thereof specifically bind to human FRα. In preferred embodiments, the ADC anti-FRα antibody or antigen-binding fragments thereof specifically bind to human FRα and cynomolgus monkey FRα.

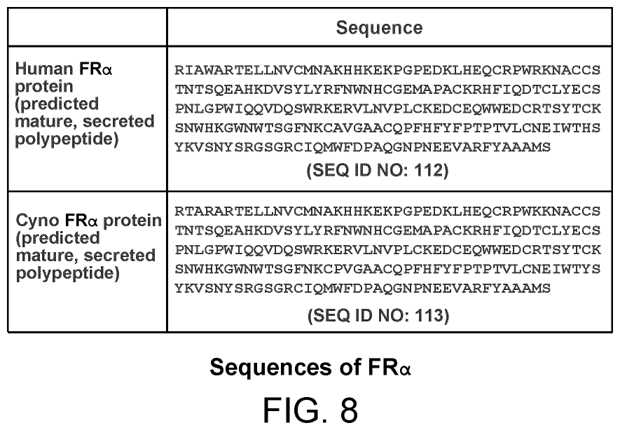

In some embodiments, the FRα protein has the sequence of SEQ ID NO: 112 or SEQ ID NO: 113, as listed in FIG. 8. In one embodiment, FRα has the sequence of SEQ ID NO: 112 and is a human FRα protein (predicted mature, secreted polypeptide). In another embodiment, FRα has the sequence of SEQ ID NO: 113 and is a cyno FRα protein (predicted mature, secreted polypeptide).

In some embodiments, the ADC antibody or antigen-binding fragment thereof does not bind to one or more selected from a mouse FRα (UniProt ID: P35846), rat FRα (UniProt ID: G3V8M6), human FRS (UniProt ID: P14207), human FRγ (UniProt ID: P41439), or a combination thereof.

The term “does not bind” means that the ADC antibody or antigen-binding fragment thereof does not substantially bind to one of more of said molecules (e.g., mouse FRα, rat FRα, human FRβ, human FRγ, or a combination thereof). The term “substantially no” when used in the context of binding herein may mean less than 5%, 2%, 1%, 0.5% or 0.1% of cells expressing one or more of said molecules in a cell culture become bound by the ADC antibody or antigen-binding fragment thereof (upon contact therewith). Suitably, the term “substantially no” when used in the context of binding herein may mean no such cells become bound.

### D. Antibody Preparation

The ADC antibody or antigen-binding fragment thereof (e.g., as monoclonal antibodies) can be made using recombinant DNA methods as described in U.S. Pat. No. 4,816,567, which is incorporated herein by reference. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen-binding fragments thereof of the desired species can be isolated from phage display libraries expressing CDRs of the desired species as described in McCafferty et al., Nature 348:552-554 (1990); Clackson et al., Nature, 352:624-628 (1991); and Marks et al., J. Mol. Biol. 222:581-597 (1991).

Affinity maturation strategies and chain shuffling strategies are known in the art and can be employed to generate high affinity human antibodies or antigen-binding fragments thereof. See Marks et al., BioTechnology 10:779-783 (1992), incorporated by reference in its entirety.

Various techniques are known for the production of antibody fragments. Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In some embodiments, ADC antibody fragments are produced recombinantly. Fab, Fv, and scFv antibody fragments can all be expressed in and secreted from E. coli or other host cells, thus allowing the production of large amounts of these fragments. Such ADC antibody fragments can also be isolated from the antibody phage libraries discussed above. The ADC antibody fragments can also be linear antibodies as described in U.S. Pat. No. 5,641,870, which is incorporated herein by reference. Other techniques for the production of antibody fragments will be apparent to the skilled practitioner.

Techniques can also be adapted to produce single-chain antibodies specific to FRα. See, e.g., U.S. Pat. No. 4,946,778. In addition, methods can be adapted for the construction of Fab expression libraries to allow rapid and effective identification of monoclonal Fab fragments with the desired specificity for FRα, or derivatives, fragments, analogs or homologs thereof. See, e.g., Huse et al., Science 246:1275-1281 (1989). Antibody fragments can be produced by techniques known in the art including, but not limited to: F(ab′)2 fragment produced by pepsin digestion of an antibody molecule; Fab fragment generated by reducing the disulphide bridges of an F(ab′)2 fragment; Fab fragment generated by the treatment of the antibody molecule with papain and a reducing agent; or Fv fragments.

### III. Linkers of the ADC

The ADC antibody or antigen-binding fragment thereof may be conjugated to an ADC payload (e.g., a cytotoxic agent or cytotoxin) by a linker.

The term “Linker” or “Spacer” as used herein means a divalent chemical moiety comprising a covalent bond or a chain of atoms that covalently attaches an ADC antibody or antigen-binding fragment thereof to an ADC payload (e.g., cytotoxin) to form an ADC. In some embodiments, the linker or spacer is a peptide spacer. In some embodiments, the linker or spacer is a non-peptide (e.g., chemical) spacer. Suitable linkers have two reactive termini, one for antibody conjugation and the other for ADC payload (e.g., cytotoxin) conjugation. Because of the formation of bonds between the linker and/or the ADC payload (e.g., cytotoxin), and between the linker and/or the ADC antibody or antigen-binding fragment thereof, one or both of the reactive termini will be absent or incomplete (such as being only the carbonyl of the carboxylic acid). These conjugation reactions are discussed in more detail below.

In preferred embodiments, the linker is attached (e.g., conjugated) in a cleavable manner to an amino residue, for example, an amino acid of an ADC antibody or antigen-binding fragment described herein.

In some embodiments, the linker is cleavable under intracellular circumstances, allowing the drug unit to be released from the ADC antibody in the intracellular environment.

Alternatively, the linker unit may not be cleavable. In such embodiments the drug is released, for example, by antibody degradation. However, non-cleavable payloads require complete mAb digestion in the lysosome and the resulting drug-containing product may be too polar, e.g., for achieving bystander effect.

The ADC is preferably stable and intact before being transported or delivered into a cell, i.e., the antibody should be attached to the drug moiety. Outside the target cell, the linkers are stable, but inside the cell, they can be cleaved at a high rate. An effective linker will: (i) maintain the antibody's specific binding properties; (ii) allow intracellular delivery of the conjugate or drug moiety; (iii) remain stable and intact, i.e., not cleaved, until the conjugate has been delivered or transported to its targeted site; and (iv) maintain the cytotoxic moiety's cell-killing or cytostatic effect. Standard analytical methods such as mass spectroscopy, HPLC, and the separation/analysis technique LC/MS can be used to assess the stability of the ADC.

The linkers may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

Linkers hydrolysable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661). Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene) (See, e.g., Thorpe et al., 1987, Cancer Res. 47:5924-5931; Wawrzynczak et al., In Immunoconjugates: Antibody Conjugates in Radioimaging and Therapy of Cancer (C. W. Vogel ed., Oxford U. Press, 1987)).

In preferred embodiments, the linker is susceptible to enzymatic hydrolysis. Such linkers are preferred over pH sensitive cleavable linkers, which may not be stable enough and cleave prematurely before reaching the target cell, and thus potential off-target toxicity may be observed. The enzymatically cleavable linker can be, e.g., a peptide-containing linker that is cleaved by an intracellular peptidase or protease enzyme, including, but not limited to, a lysosomal or endosomal protease. One benefit of employing intracellular proteolytic release of the therapeutic drug is that the agent is usually attenuated when conjugated, and the conjugates' serum stabilities are usually high. In some embodiments, the peptidyl linker is at least two amino acids long or at least three amino acids long. Exemplary amino acid linkers include a dipeptide, a tripeptide, a tetrapeptide or a pentapeptide. Peptides comprising the amino acids valine, alanine, citrulline (Cit), phenylalanine, lysine, leucine, and glycine are examples of appropriate peptides. Natural amino acids, minor amino acids, and non-naturally occurring amino acid analogs, such as citrulline, are all examples of amino acid residues that make up an amino acid linker component. Exemplary dipeptides include valine-citrulline (VC or Val-Cit) and alanine-phenylalanine (AF or Ala-Phe). Exemplary tripeptides include glycine-valine-citrulline (Gly-Val-Cit) and glycine-glycine-glycine (Gly-Gly-Gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit.

In some embodiments, the linker comprises PEG. A stable protease-cleavable linker containing PEG can limit payload hydrophobicity and be able to selectively cleave and release the free drug inside target cancer cells. A less hydrophobic nature of the linker as described herein can enable high loading of the drug onto the antibody or antigen-binding fragment (e.g., DAR8) without aggregation, which would be significantly higher than mirvetuximab soravtansine (DAR3-4) or derivatives thereof, such as IMGN151 (DAR3.5). This could allow the ADC to deliver a significantly higher concentration of ADC payload (e.g., cytotoxin) to the target cancer cells via binding to FRα on the cancer cells.

In some embodiments, the linker comprises maleimide. The use of maleimide in the linker may allow the generation of DAR8 and DAR4 ADCs by making use of the native interchain disulphides in the antibodies. This is advantageous over the conjugation of surface amines from lysine residues which could result in a mixture of DAR species and batch-to-batch variability. There may also be reproducibility issues that affect ADC efficacy if conjugation sites interfere with antigen binding. Moreover, other conjugation methods, e.g., azide-alkyne click chemistry involving an engineered antibody may not easily achieve a DAR of more than four.

In certain embodiments, the ADC anti-FRα antibody or antigen-binding fragment thereof is linked to an ADC payload (e.g., cytotoxin), via a linker R^{L}. In some embodiments, R^{L} is selected from:

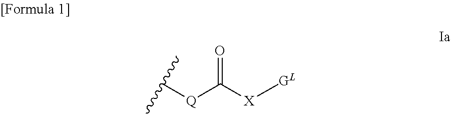

wherein Q is:

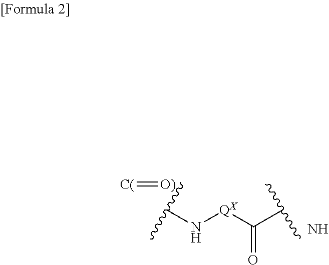

where Q^{X} is such that Q is an amino-acid residue, a dipeptide residue, a tripeptide residue or a tetrapeptide residue, and X in Formula 1 is:

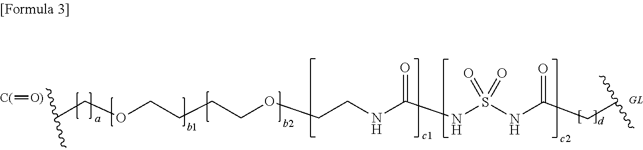

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0). G^{L} in Formula 1 is a linker for connecting to an ADC antibody or antigen-binding fragment thereof. Alternatively, G^{L} in Formula 1 is the compound denoted as “Ib”:

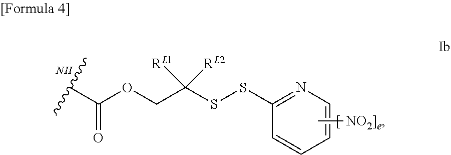

where R^{L1} and R^{L2} are independently selected from H and methyl, or together with the carbon atom to which they are bound form a cyclopropylene or cyclobutylene group; and e is 0 or 1; or the compound denoted as “Ib′”:

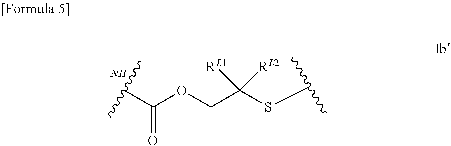

where R^{L1} and R^{L2} are as defined above.

By way of example, preferred embodiments of G^{L}, X, Q^{X} (e.g., within the linker of Ia of Formula 1) and the linker of Ib of Formula 4 will be outlined.

The following preferences may apply to all embodiment or may relate to a single embodiment. The preferences may be combined together in any combination.

Various definitions that pertain to certain terms in this section are provided under the heading “Chemical Definitions” provided below.

G^{L} of the linker of Formula 1 may be selected from:

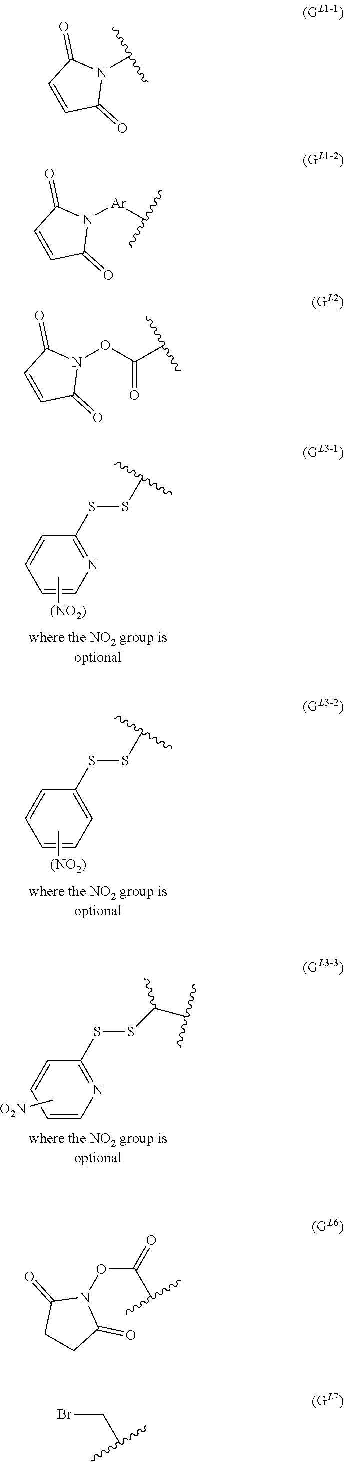

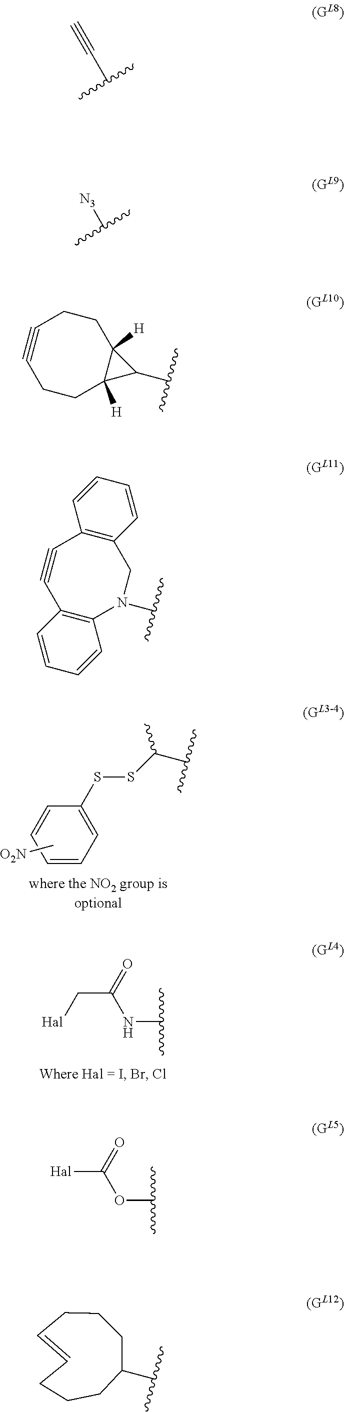

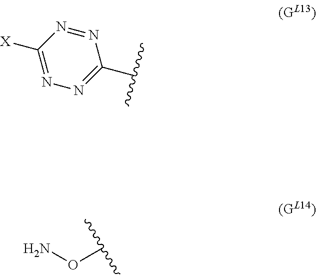

where Ar represents a C₅₋₆ arylene group, e.g., phenylene, and X represents C₁₋₄ alkyl.

In some embodiments, G^{L} is selected from G^{L1-1} and G^{L1-2}. In some of these embodiments, G^{L} is G^{L1-1}.

X in Formula 1 is preferably:

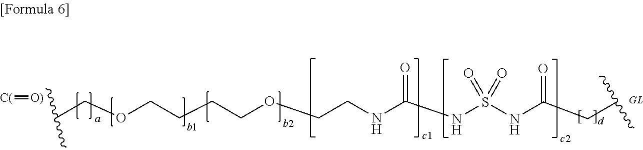

where a=0 to 5, b1=0 to 16, b2=0 to 16, c=0 or 1, d=0 to 5, wherein at least b1 or b2=0 and at least c1 or c2=0.
“a” may be 0, 1, 2, 3, 4 or 5. In some embodiments, a is 0 to 3. In some of these embodiments, a is 0 or 1. In further embodiments, a is 0.
“b1” may be 0, 1, 2, 3, 4, 5, 6, 7, 8, 9, 10, 11, 12, 13, 14, 15 or 16. In some embodiments, b1 is 0 to 12. In some of these embodiments, b1 is 0 to 8, and may be 0, 2, 3, 4, 5 or 8.
“b2” may be 0, 1, 2, 3, 4, 5, 6, 7, 8, 9, 10, 11, 12, 13, 14, 15 or 16. In some embodiments, b2 is 0 to 12. In some of these embodiments, b2 is 0 to 8, and may be 0, 2, 3, 4, 5 or 8. Preferably, only one of b1 and b2 may not be 0.
“c1” may be 0 or 1. c2 may be 0 or 1. Preferably, only one of c1 and c2 may not be 0.
“d” may be 0, 1, 2, 3, 4 or 5. In some embodiments, d is 0 to 3. In some of these embodiments, d is 1 or 2. In further embodiments, d is 2. In further embodiments, d is 5.

In some embodiments of X, a is 0, b1 is 0, cl is 1, c2 is 0 and d is 2, and b2 may be from 0 to 8. In some of these embodiments, b2 is 0, 2, 3, 4, 5 or 8. In some embodiments of X, a is 1, b2 is 0, cl is 0, c2 is 0 and d is 0, and b1 may be from 0 to 8. In some of these embodiments, b1 is 0, 2, 3, 4, 5 or 8. In some embodiments of X, a is 0, b1 is 0, cl is 0, c2 is 0 and d is 1, and b2 may be from 0 to 8. In some of these embodiments, b2 is 0, 2, 3, 4, 5 or 8. In some embodiments of X, b1 is 0, b2 is 0, cl is 0, c2 is 0 and one of a and d is 0. The other of a and d is from 1 to 5. In some of these embodiments, the other of a and d is 1. In other of these embodiments, the other of a and d is 5. In some embodiments of X, a is 1, b2 is 0, cl is 0, c2 is 1, d is 2, and b1 may be from 0 to 8. In some of these embodiments, b2 is 0, 2, 3, 4, 5 or 8.

In some embodiments, Q of Formula 1 is an amino acid residue. The amino acid may be a natural amino acid or a non-natural amino acid. For example, Q may be selected from: Phe, Lys, Val, Ala, Cit, Leu, Ile, Arg, and Trp, where Cit is citrulline.

In some embodiments, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In some embodiments, Q is selected from:
^{NH}-Phe-Lys-^{C═O},
^{NH}-Val-Ala-^{C═O},
^{NH}-Val-Lys-^{C═O},
^{NH}-Ala-Lys-^{C═O},
^{NH}-Val-Cit-^{C═O},
^{NH}-Phe-Cit-^{C═O},
^{NH}-Leu-Cit-^{C═O},
^{NH}-Ile-Cit-^{C═O},
^{NH}-Phe-Arg-^{C═O},
^{NH}-Trp-Cit-^{C═O}, and
^{NH}-Gly-Val-^{C═O};
where Cit is citrulline. Preferably, Q is selected from:
^{NH}-Phe-Lys-^{C═O},
^{NH}-Val-Ala-^{C═O},
^{NH}-Val-Lys-^{C═O},
^{NH}-Ala-Lys-^{C═O}, and
^{NH}-Val-Cit-^{C═O}.

More preferably, Q is selected from ^{NH}-Phe-Lys-^{C═O}, ^{NH}_Val-Cit-^{C═O} or ^{NH}-Val-Ala-^{C═O}. Other suitable dipeptide combinations include:
^{NH}-Gly-Gly-^{C═O},
^{NH}-Gly-Val-^{C═O},
^{NH}-Pro-Pro-^{C═O} and
^{NH}-Val-Glu-^{C═O}.

Other dipeptide combinations may be used, including those described by Dubowchik et al., Bioconjugate Chemistry, 2002, 13, 855-869, which is incorporated herein by reference.

In some embodiments, Q is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:
^{NH}-Glu-Val-Ala-^{C═O},
^{NH}-Glu-Val-Cit-^{C═O},
^{NH}-αGlu-Val-Ala-^{C═O}, and
^{NH}-αGlu-Val-Cit-^{C═O}.

In some embodiments, Q is a tetrapeptide residue. The amino acids in the tetrapeptide may be any combination of natural amino acids and non-natural amino acids. In some embodiments, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:
^{NH}-Gly-Gly-Phe-Gly^{C═O}; and
^{NH}-Gly-Phe-Gly-Gly^{C═O}.

In some embodiments, the tetrapeptide is:
^{NH}-Gly-Gly-Phe-Gly^{C═O}.

In the above representations of peptide residues, ^{NH}- represents the N-terminus, and -^{C═O} represents the C-terminus of the residue. The C-terminus binds to the NH of the “Drug Unit” (e.g., A* in Formula 14 below). Glu represents the residue of glutamic acid, i.e.:

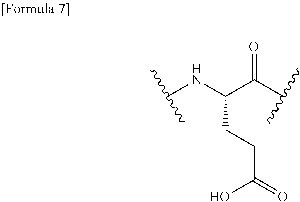 [SMILES: *N[C@@H](CCC(=O)O)C(*)=O]

αGlu represents the residue of glutamic acid when bound via the α-chain, i.e.:

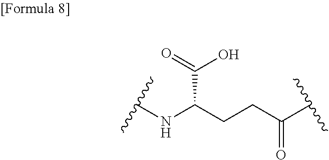 [SMILES: *N[C@@H](CCC(*)=O)C(=O)O]

In some embodiments, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

R^{L1} and R^{L2} of the linker Ib shown in Formula 4 may be independently selected from H and methyl, or together with the carbon atom to which they are bound form a cyclopropylene or cyclobutylene group.

In some embodiments, both R^{L1} and R^{L2} are H. In some embodiments, R^{L1} is H and R^{L2} is methyl. In some embodiments, both R^{L1} and R^{L2} are methyl.

In some embodiments, R^{L1} and R^{L2} together with the carbon atom to which they are bound form a cyclopropylene group. In some embodiments, R^{L1} and R^{L2} together with the carbon atom to which they are bound form a cyclobutylene group.

In the group Ib of Formula 4, in some embodiments, e is 0. In other embodiments, e is 1 and the nitro group may be in any available position of the ring. In some of these embodiments, it is in the ortho position. In others of these embodiments, it is in the para position.

In some embodiments, the linker R^{L} shown in Formula 1 is selected from:

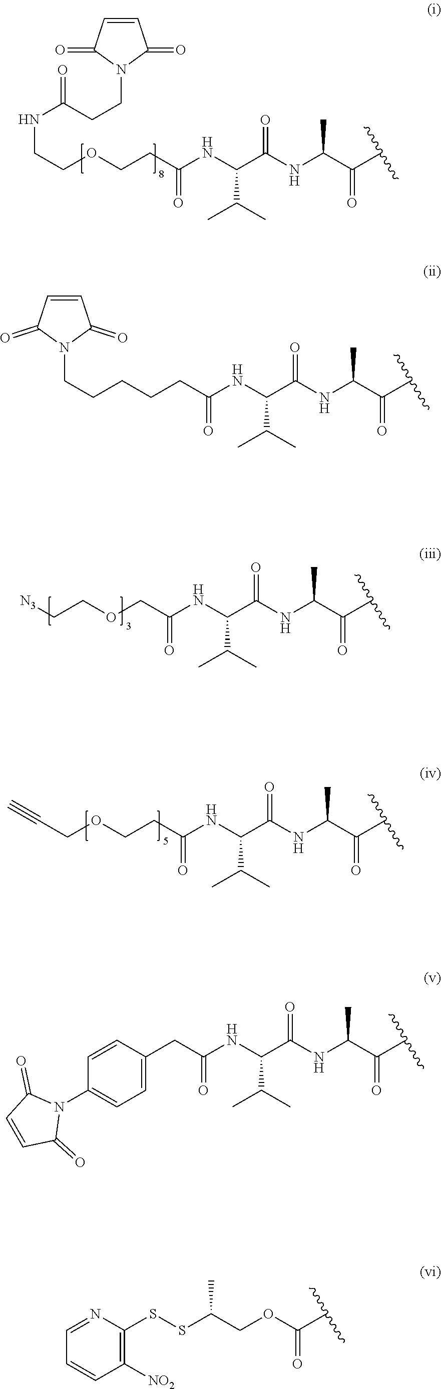

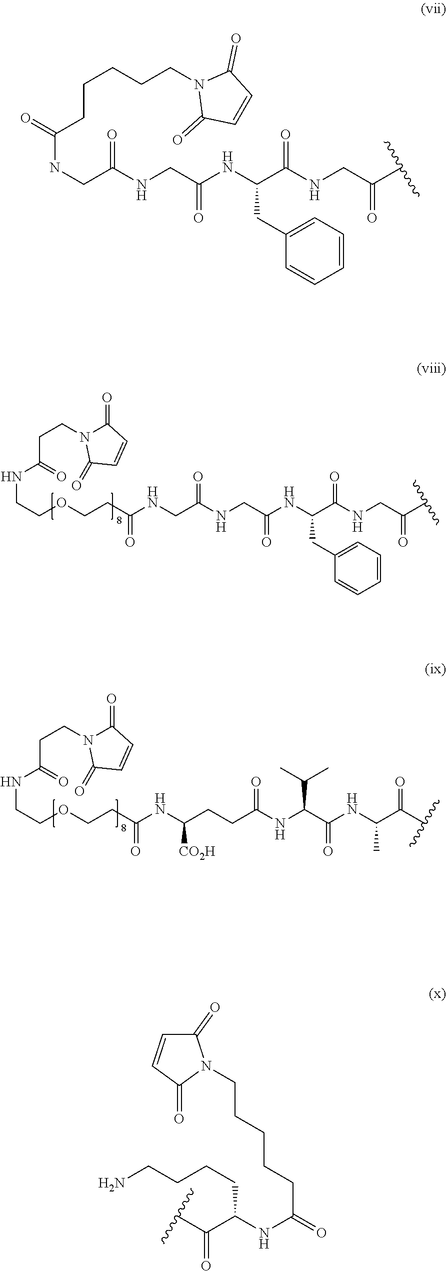

Preferably, R^{L} is:

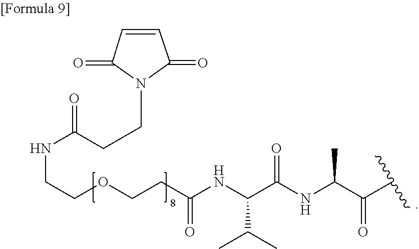

For example, the ADC may be of the general formula:

L-(D^{L})ₚ [Formula 10]
or a pharmaceutically acceptable salt or solvate thereof, wherein L is an ADC antibody or antigen-binding fragment thereof, D^{L} is a “Drug Unit” (or an ADC payload) (e.g., cytotoxin such as TOPOi) having a linker R^{LL} connected to the ADC antibody or antigen-binding fragment thereof. In some embodiments, the linker R^{LL} is preferably selected from “Ia′”:

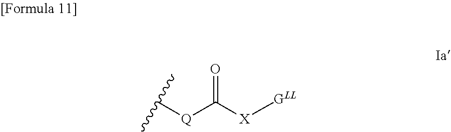

where Q and X are as defined above and G^{LL} is a linker connected to an ADC antibody or antigen-binding fragment thereof. Ib′ is represented by the formula:

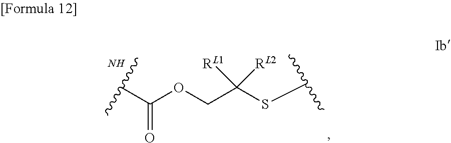

where R^{L1} and R^{L2} are as defined above.

The “p” in L-(D^{L})ₚ of Formula 10 for the antibody-drug conjugate represents the drug loading and is the number of “Drug Units” (e.g., cytotoxin such as TOPOi) per antibody or antigen-binding fragment thereof. The drug loading ranges from 1 to 20 Drug units (D) per antibody or antigen-binding fragment thereof. For compositions, “p” represents the average drug loading of the conjugates in the composition, and “p” ranges from 1 to 20. In some embodiments, the range of “p” is selected from 1 to 10, 2 to 10, 2 to 8, 2 to 6, and 4 to 10; preferably p is 8.

G^{LL} of Formula 11 may be selected from:

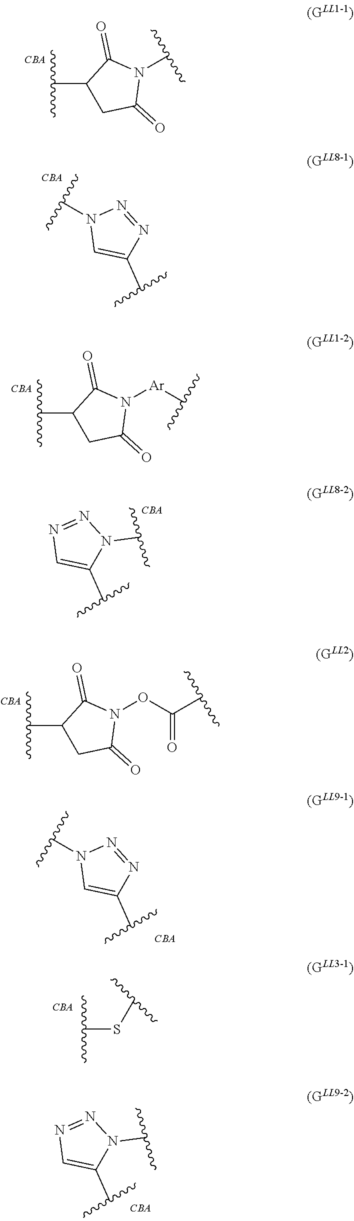

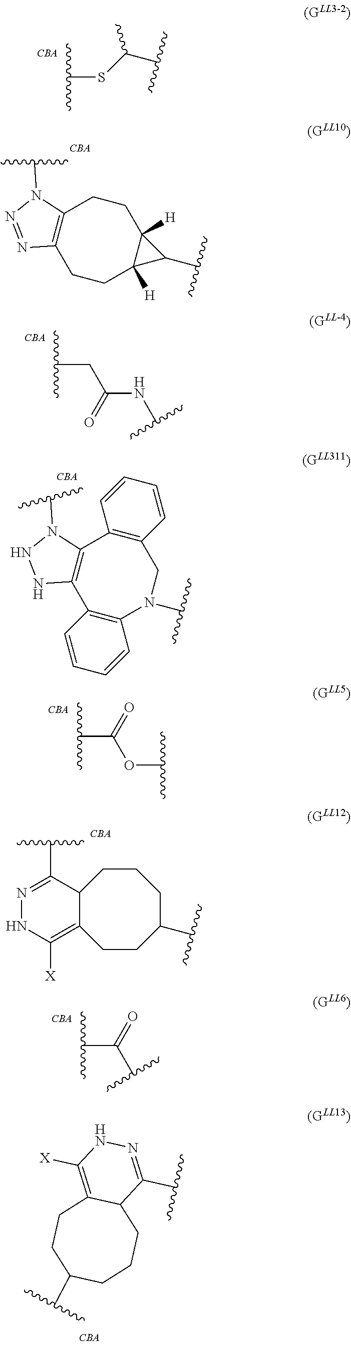

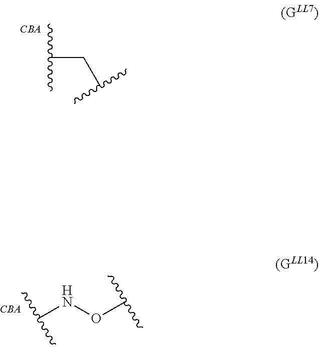

where Ar represents a C₅₋₆ arylene group, e.g., phenylene and X represents C₁₋₄ alkyl.

In some embodiments, G^{LL} is selected from G^{LL1-1} and G^{LL1-2}. In some of these embodiments, G^{LL} is G^{LL1-1}.

In some embodiments, R^{LL} is a group derived from the R^{L} groups above.

It will be recognized by one of skill in the art that any one or more of the chemical groups, moieties and features disclosed herein may be combined in multiple ways to form linkers useful for conjugation of the ADC antibodies and ADC payloads (e.g., cytotoxins) as disclosed herein.

In some embodiments where compounds described herein are provided in a single enantiomer or in an enantiomerically enriched form, the enantiomerically enriched form has an enantiomeric ratio greater than 60:40, 70:30; 80:20 or 90:10. In further embodiments, the enantiomeric ratio is greater than 95:5, 97:3 or 99:1.

### IV. Further Chemical Definitions

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g., C₅₋₆) denote the number of ring atoms, or range of number of ring atoms, whether carbon atoms or heteroatoms. The ring atoms may be all carbon atoms, as in “carboarylene groups”, in which case the group is phenylene (C₆). Alternatively, the ring atoms may include one or more heteroatoms, as in “heteroarylene groups”. Examples of heteroarylene groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and
N₃: triazole (C₅), triazine (C₆).

C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g., partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃) and butyl (C₄). Examples of saturated linear alkyl groups include, but are not limited to, methyl (C₁), ethyl (C₂), n-propyl (C₃) and n-butyl (C₄). Examples of saturated branched alkyl groups include iso-propyl (C₃), iso-butyl (C₄), sec-butyl (C₄) and tert-butyl (C₄).

C₂₋₄ Alkenyl: The term “C₂₋₄ alkenyl” as used herein, pertains to an alkyl group having one or more carbon-carbon double bonds. Examples of unsaturated alkenyl groups include, but are not limited to, ethenyl (vinyl, —CH═CH₂), 1-propenyl (—CH═CH-CH₃), 2-propenyl (allyl, —CH-CH═CH₂), isopropenyl (1-methylvinyl, —C(CH₃)═CH₂) and butenyl (C₄).

C₂₋₄ alkynyl: The term “C₂₋₄ alkynyl” as used herein, pertains to an alkyl group having one or more carbon-carbon triple bonds. Examples of unsaturated alkynyl groups include, but are not limited to, ethynyl (—C≡CH) and 2-propynyl (propargyl, —CH₂—C≡CH).

C₃₋₄ cycloalkyl: The term “C₃₋₄ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms. Examples of cycloalkyl groups include, but are not limited to, those derived from: saturated monocyclic hydrocarbon compounds, such as cyclopropane (C₃) and cyclobutane (C₄); and unsaturated monocyclic hydrocarbon compounds, such as cyclopropene (C₃) and cyclobutene (C₄).

Connection labels: In the formula:

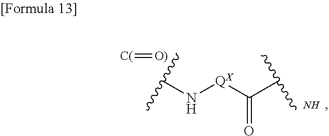

the superscripted labels ^{C═O} and ^{NH} indicate the group to which the atoms are bound. For example, the NH group is shown as being bound to a carbonyl (which is not part of the moiety illustrated), and the carbonyl is shown as being bound to a NH group (which is not part of the moiety illustrated).

### V. Cytotoxin of ADC

The anti-FRα ADC used in the novel methods disclosed herein comprises an ADC payload and an ADC anti-FRαantibody or antigen-binding fragment thereof. In a preferred embodiment, the anti-FRα ADC comprises an ADC anti-FRα antibody or antigen-binding fragment thereof conjugated to one or more cytotoxins.

In some embodiments, the number of ADC payloads (e.g., cytotoxins) per antibody (or antigen-binding fragment thereof) can be expressed as a ratio of ADC payload (i.e., drug) to antibody. This ratio is referred to as the Drug-to-Antibody Ratio (DAR). The DAR is the average number of drugs (i.e., ADC payload) linked to each antibody. In some embodiments, the DAR is in the range of about 1 to 20. In some embodiments, the range of DAR is selected from about 1 to 10, about 2 to 10, about 2 to 8, about 2 to 6, and about 4 to 10. In preferred embodiments, the DAR is about 4 (e.g., 3.8-4.2) or about 8 (e.g., 7.6-8.4), more preferably about 8 (e.g., 7.6-8.4).

The cytotoxin can be any molecule known in the art that inhibits or prevents the function of cells and/or causes destruction of cells (cell death), and/or exerts anti-neoplastic/anti-proliferative effects. A number of classes of cytotoxic agents are known to have potential utility in ADC molecules. Suitable cytotoxic agents for use in ADCs include, but are not limited to, topoisomerase I inhibitors (TOPOi), amanitins, auristatins, daunomycins, doxorubicins, duocarmycins, dolastatins, enediynes, lexitropsins, taxanes, puromycins, maytansinoids, vinca alkaloids, tubulysins and pyrrolobenzodiazepines (PBDs). Examples of such cytotoxic agents are AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, vinblastine, methotrexate, and netropsin, and derivatives and analogs thereof. Additional disclosure regarding cytotoxins suitable for use in ADCs can be found, for example, in International Patent Application Publication Nos. WO 2015/155345 and WO 2015/157592, incorporated herein by reference in their entirety.

In some embodiments, the ADC comprises an anti-FRα antibody or antigen-binding fragment thereof conjugated to one or more cytotoxin selected from a topoisomerase I inhibitor, tubulysin derivative, a pyrrolobenzodiazepine, or a combination thereof. For example, the ADC antibody or antigen binding fragment thereof is conjugated to one or more cytotoxins selected from the group consisting of topoisomerase I inhibitor SG3932 (also known as AZ14170133), SG4010, SG4057 or SG4052 (the structures of which are provided below), or a combination thereof.

It is preferred that the ADC antibody or antigen-binding fragment thereof is conjugated to a topoisomerase I inhibitor, more preferably the topoisomerase I inhibitor SG3932.

Topoisomerase inhibitors are chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle. Topoisomerase I inhibitors are advantageous as they mediate highly effective tumor cell killing with fewer toxicities to the patient. In particular, alternative payloads such as microtubule inhibitor that have generally been used to-date for the development of anti-FRα ADCs are known to have toxicity problems (Hinrichs, et al. AAPS J. 2015 September; 17(5): 1055-1064). Moreover, the use of a less hydrophobic linker with a less potent warhead (e.g., TOPOi) would facilitate bystander killing in heterogeneous tumors. Although bystander activity may be achieved by increasing the potency and/or improving warhead permeability through increased hydrophobicity, this may result in increased toxicity due to non-specific uptake.

A general example of a suitable topoisomerase I inhibitor is represented by the following compound:

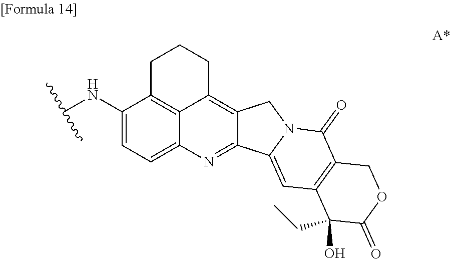 [SMILES: *Nc1ccc2nc3c(c4c2c1CCC4)Cn1c-3cc2c(c1=O)COC(=O)[C@]2(O)CC]

The compound shown above is denoted as A*, and may be referred to as a “Drug Unit” herein. The compound A* is preferably provided with a linker for connecting (preferably conjugating) to an ADC antibody or antigen-binding fragment. In preferred embodiments, the linker is attached (e.g., conjugated) in a cleavable manner to an amino residue, for example, an amino acid of an ADC antibody or antigen-binding fragment.

More particularly, an example of a suitable topoisomerase I inhibitor is represented by the following compound “I” with the formula:

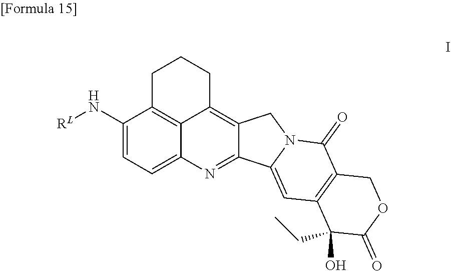

The compound I includes salts and solvates thereof, wherein R^{L} is a linker for connection to an ADC antibody or antigen binding fragment thereof described herein. In some embodiments, R^{L} is as described above.

Accordingly, the ADC used in the novel methods may have the general formula:

L-(D^{L})ₚ [Formula 10]
or a pharmaceutically acceptable salt or solvate thereof, wherein L and p are defined above, D^{L} is a topoisomerase I inhibitor having a linker (e.g., Drug Linker unit) of compound III with the formula:

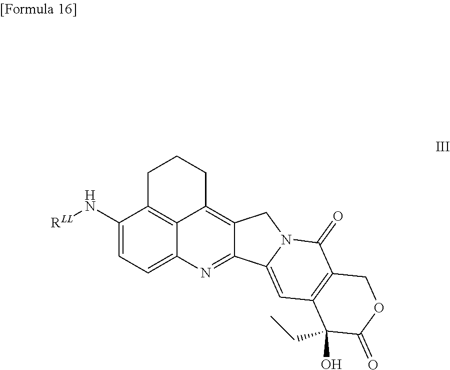

Compound III includes salts and solvates thereof, wherein R^{LL} is a linker for connection to an ADC antibody or antigen binding fragment thereof described herein. In some embodiments, R^{LL} is defined as above.

In some embodiments, the compound I of Formula 15 is of the type I^{P} with the formula:

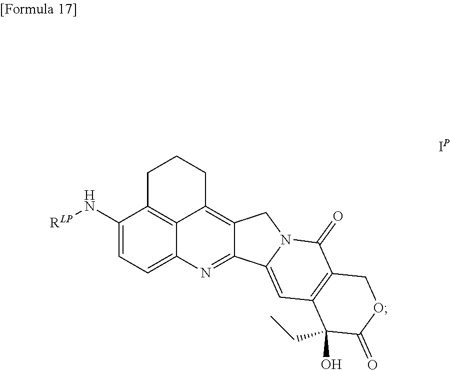

The compound I^{P} includes salts and solvates thereof, wherein R^{LP} is a linker for connection to an ADC antibody or antigen-binding fragment thereof, wherein said linker is selected from the compound Ia^{P} with the formula:

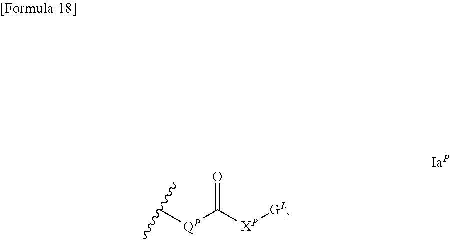

wherein Q^{P} is:

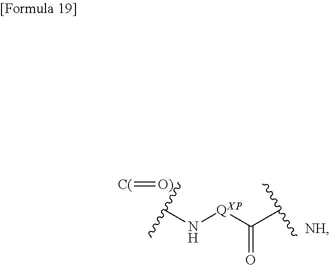

where Q^{XP} is such that Q^{P} is an amino-acid residue, a dipeptide residue or a tripeptide residue. X^{P} in Formula 18 is:

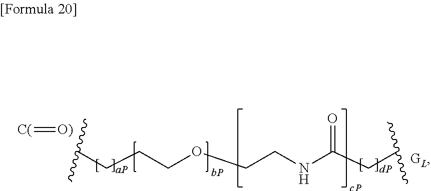

where aP=0 to 5, bP=0 to 16, cP=0 or 1, dP=0 to 5. Alternatively, G^{L} in Formula 18 is the compound Ib with the formula:

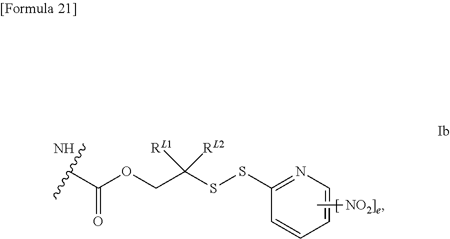

where R^{L1} and R^{L2} are independently selected from H and methyl, or together with the carbon atom to which they are bound form a cyclopropylene or cyclobutylene group; and e is 0 or 1. aP in Formula 20 may be 0, 1, 2, 3, 4 or 5. In some embodiments, aP is 0 to 3. In some of these embodiments, aP is 0 or 1. In further embodiments, aP is 0. bP in Formula 20 may be 0, 1, 2, 3, 4, 5, 6, 7, 8, 9, 10, 11, 12, 13, 14, 15 or 16. In some embodiments, b is 0 to 12. In some of these embodiments, bP is 0 to 8, and may be 0, 2, 4 or 8. cP in Formula 20 may be 0 or 1. dP in Formula 20 may be 0, 1, 2, 3, 4 or 5. In some embodiments, dP is 0 to 3. In some of these embodiments, dP is 1 or 2. In further embodiments, dP is 2.

In some embodiments of X^{P} of Formula 18, aP is 0, cP is 1 and dP is 2, and bP may be from 0 to 8. In some of these embodiments, bP is 0, 4 or 8.

The preferences for Q^{X} of Formula 13 for compound I of Formula 15 may apply to Q^{XP} of Formula 19 (for example, where appropriate).

The preferences for G^{L}, R^{L1}, R^{L2} and e above for compound I of Formula 15 may apply to compound I^{P} of Formula 17.

In some embodiments, the conjugate of the ADC of Formula 10 is the ADC^{P} with the formula:

L-(D^{LP})ₚ [Formula 22]
or a pharmaceutically acceptable salt or solvate thereof, wherein L is an ADC antibody or antigen-binding fragment thereof, D^{LP} is a topoisomerase I inhibitor (e.g. Drug Linker unit) designated as the compound III^{P} with the formula:

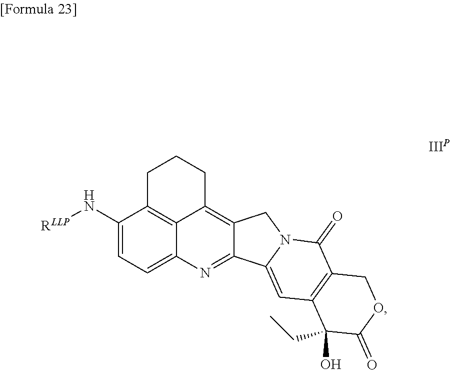

where R^{LLP} is a linker connected to the ADC antibody or antigen-binding fragment thereof, wherein said linker is selected from the group Ia^{P}′ and Ib′. The group Ia^{P}′ is represented by the formula:

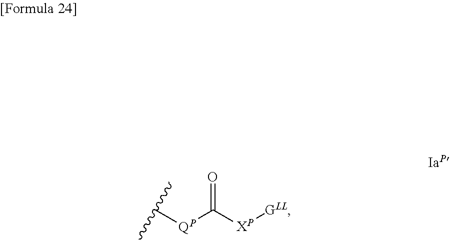

where Q^{P}, X^{P} and G^{LL} are as defined above. The group Ib′ is represented by the formula:

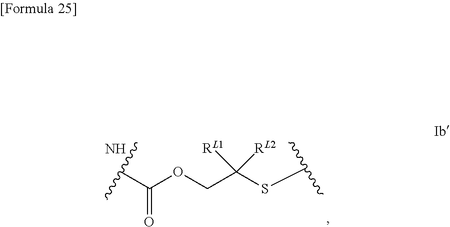

where R^{L1} and R^{L2} are as defined above; and p is an integer of from 1 to 20.

In some embodiments, the compound I of Formula 15 is of the type I^{P2} with the formula:

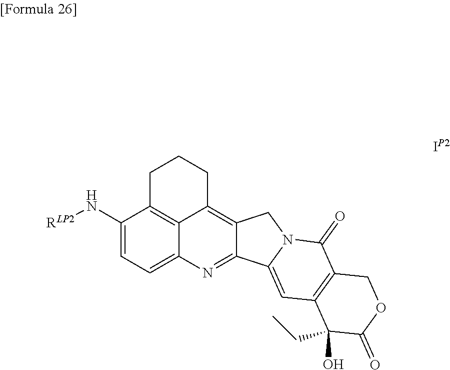

and salts and solvates thereof. R^{LP2} is a linker for connection to an ADC antibody or antigen-binding fragment thereof, wherein said linker is selected from the group Ia^{P2} and Ib. The group Ia^{P2} is represented by the formula:

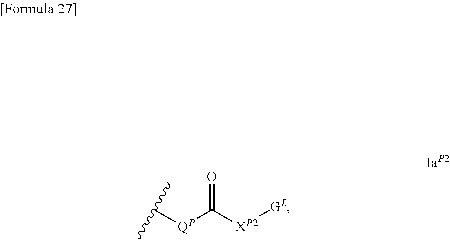

wherein Q is:

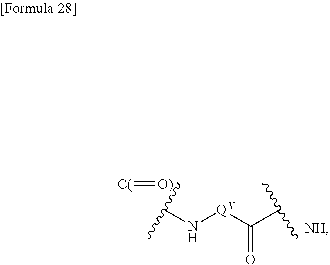

where Q^{X} is such that Q is an amino-acid residue, a dipeptide residue, a tripeptide residue or a tetrapeptide residue. X^{P2} in Formula 27 is:

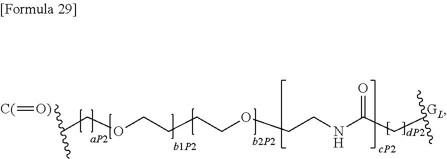

where aP2=0 to 5, b1P2=0 to 16, b2P2=0 to 16, cP2=0 or 1, dP2=0 to 5, wherein at least b1P2 or b2P2=0 (i.e., only one of b1 and b2 may not be 0). GL in Formula 27 is a linker for connecting to an ADC antibody or antigen-binding fragment thereof.

The group Ib is represented by the formula:

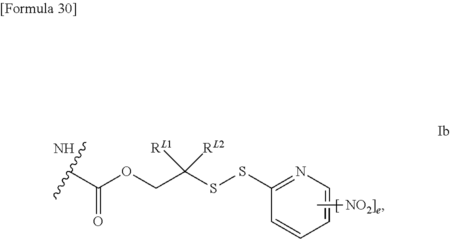

where R^{L1} and R^{L2} are independently selected from H and methyl, or together with the carbon atom to which they are bound form a cyclopropylene or cyclobutylene group; and e is 0 or 1.

In some embodiments of X^{P2} of Formula 27, aP2 may be 0, 1, 2, 3, 4 or 5. In some embodiments, aP2 is 0 to 3. In some of these embodiments, aP2 is 0 or 1. In further embodiments, aP2 is 0. In some embodiments, b1P2 may be 0, 1, 2, 3, 4, 5, 6, 7, 8, 9, 10, 11, 12, 13, 14, 15 or 16. In some embodiments, b1P2 is 0 to 12. In some of these embodiments, b1P2 is 0 to 8, and may be 0, 2, 3, 4, 5 or 8. In some embodiments, b2P2 may be 0, 1, 2, 3, 4, 5, 6, 7, 8, 9, 10, 11, 12, 13, 14, 15 or 16. In some embodiments, b2P2 is 0 to 12. In some of these embodiments, b2P2 is 0 to 8, and may be 0, 2, 3, 4, 5 or 8. Preferably, only one of b1P2 and b2P2 may not be 0. In some embodiments, cP2 may be 0 or 1. In some embodiments, dP2 may be 0, 1, 2, 3, 4 or 5. In some embodiments, dP2 is 0 to 3. In some of these embodiments, dP2 is 1 or 2. In further embodiments, dP2 is 2. In further embodiments, dP2 is 5.

In some embodiments of X^{P2}, aP2 is 0, b1P2 is 0, cP2 is 1 and dP2 is 2, and b2P2 may be from 0 to 8. In some of these embodiments, b2P2 is 0, 2, 3, 4, 5 or 8. In some embodiments of X^{P2}, aP2 is 1, b2P2 is 0, cP2 is 0 and dP2 is 0, and b1P2 may be from 0 to 8. In some of these embodiments, b1P2 is 0, 2, 3, 4, 5 or 8. In some embodiments of X^{P2}, aP2 is 0, b1P2 is 0, cP2 is 0 and dP2 is 1, and b2P2 may be from 0 to 8. In some of these embodiments, b2P2 is 0, 2, 3, 4, 5 or 8. In some embodiments of X^{P2}, b1P2 is 0, b2P2 is 0, cP2 is 0 and one of aP2 and dP2 is 0. The other of aP2 and d is from 1 to 5. In some of these embodiments, the other of aP2 and d is 1. In other of these embodiments, the other of aP2 and dP2 is 5.

The preferences for Q^{X} of Formula 2 for compound I of Formula 15 may apply to Q^{X} in group Ia^{P2} of Formula 27 (e.g. where appropriate). The preferences for G^{L}, R^{L1}, R^{L2} and e above for compound I of Formula 15 may apply to compound type I^{P2} of Formula 26.

In some embodiments, the conjugate of the ADC of Formula 10 is the compound ADC^{P2} with the formula:

L-(D^{LP2})ₚ [Formula 31]
or a pharmaceutically acceptable salt or solvate thereof, wherein L is an ADC antibody or antigen-binding fragment thereof, D^{LP2} is a topoisomerase I inhibitor (e.g., Drug Linker unit) that is the compound III^{P2} with the formula:

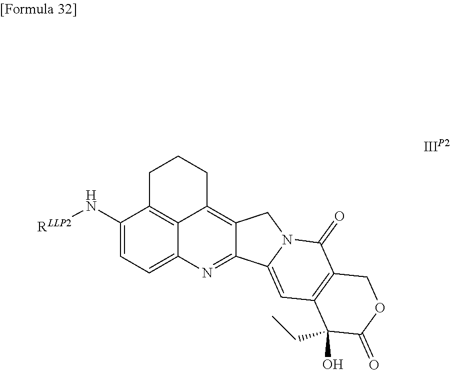

R^{LLP2} is a linker connected to the ADC antibody or antigen-binding fragment thereof, wherein said linker is selected from the group Ia^{P2}′ and Ib′. The group Ia^{P2}′ is represented by the formula:

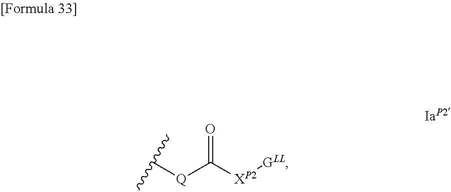

where Q and X^{P2} are as defined above and G^{LL} is a linker connected to the ADC antibody or antigen-binding fragment thereof. The group Ib′ is represented by the formula:

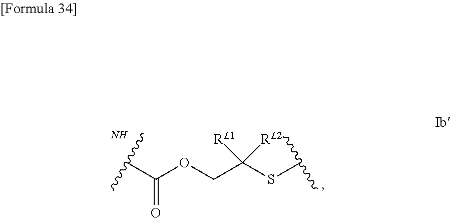

where R^{L1} and R^{L2} are as defined above; and p is an integer of from 1 to 20.

Particularly suitable topoisomerase I inhibitors include those having the following formulas:

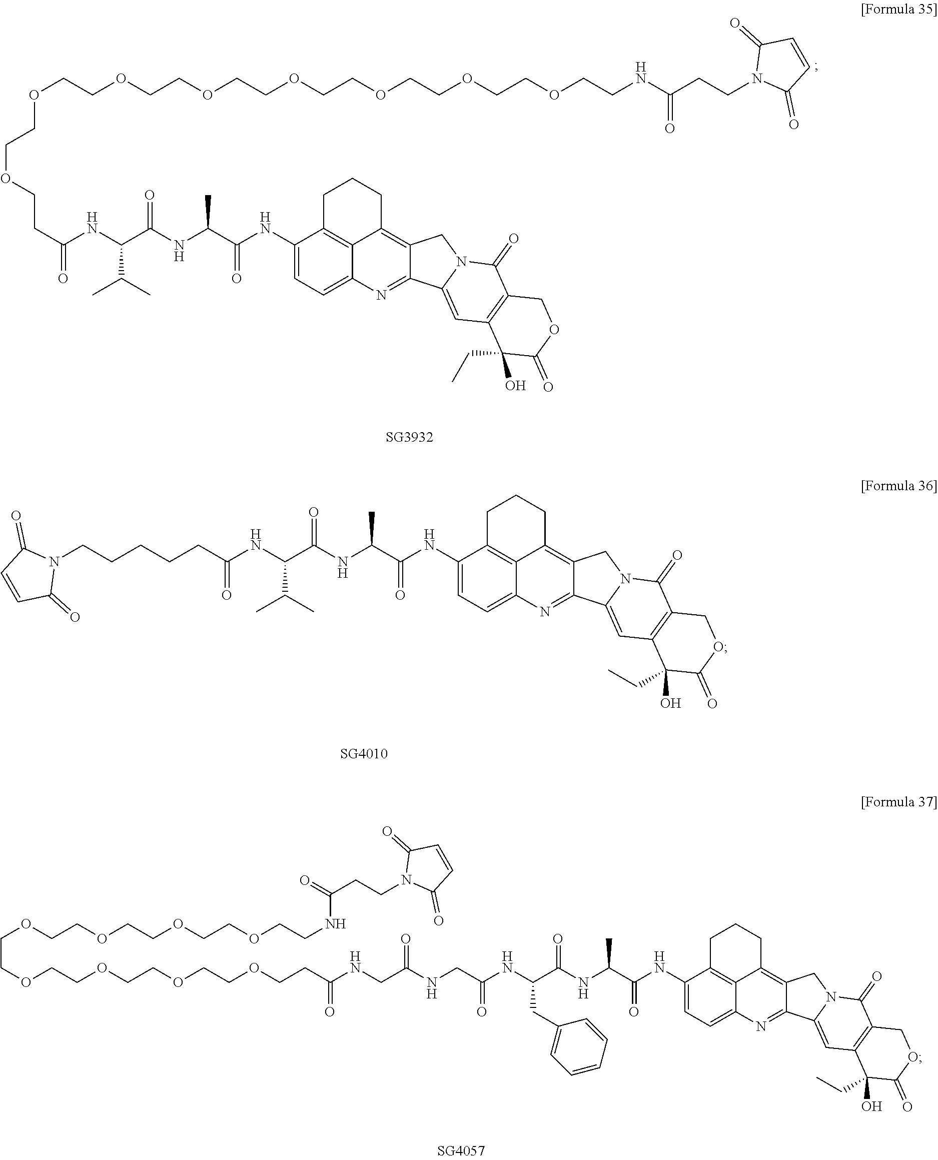 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3 ; CC[C@@]1(O)C(=O)OCc2c1cc1n(c2=O)Cc2c-1nc1ccc(NC(=O)[C@H](C)NC(=O)[C@@H](NC(=O)CCCCCN3C(=O)C=CC3=O)C(C)C)c3c1c2CCC3 ; CC[C@@]1(O)C(=O)OCc2c1cc1n(c2=O)Cc2c-1nc1ccc(NC(=O)[C@H](C)NC(=O)[C@H](Cc3ccccc3)NC(=O)CNC(=O)CNC(=O)CCOCCOCCOCCOCCOCCOCCOCCOCCNC(=O)CCN3C(=O)C=CC3=O)c3c1c2CCC3]

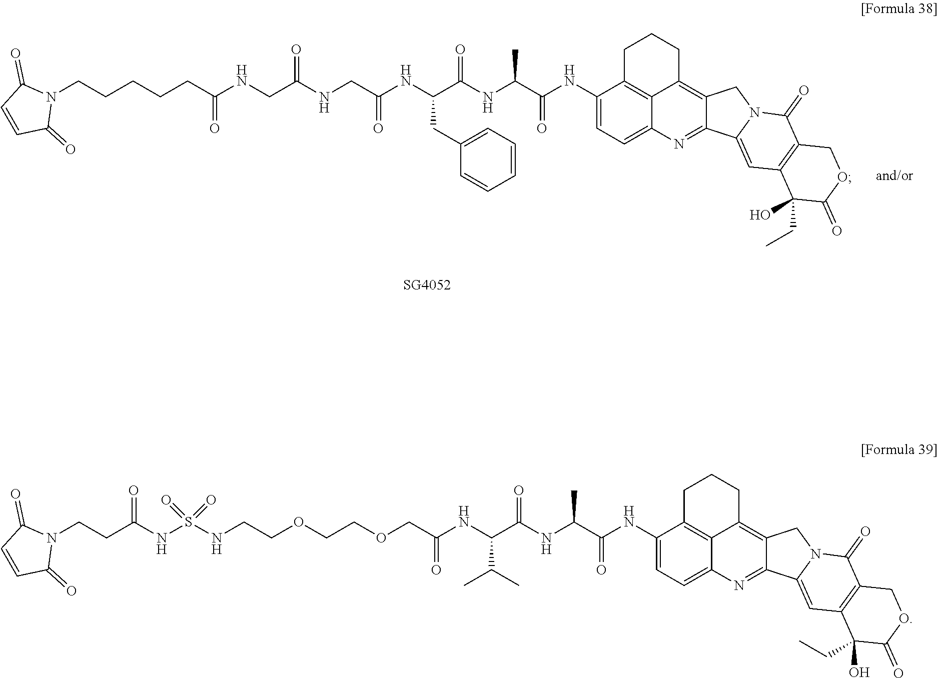 [SMILES: CC[C@@]1(O)C(=O)OCc2c1cc1n(c2=O)Cc2c-1nc1ccc(NC(=O)[C@H](C)NC(=O)[C@H](Cc3ccccc3)NC(=O)CNC(=O)CNC(=O)CCCCCN3C(=O)C=CC3=O)c3c1c2CCC3 ; CC[C@@]1(O)C(=O)OCc2c1cc1n(c2=O)Cc2c-1nc1ccc(NC(=O)[C@H](C)NC(=O)[C@@H](NC(=O)COCCOCCNS(=O)(=O)NC(=O)CCN3C(=O)C=CC3=O)C(C)C)c3c1c2CCC3]

The topoisomerase I inhibitor SG3932 is particularly preferred. Thus, in a preferable embodiment, the ADC used in the novel methods comprises an antibody or antigen-binding fragment thereof conjugated to a topoisomerase I inhibitor having the formula of SG3932):

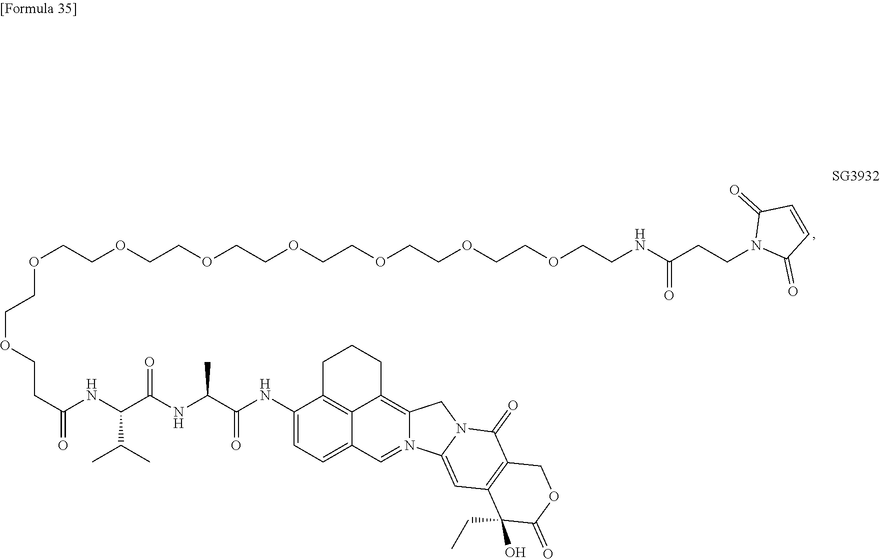

Synthetic methods of making topoisomerase I inhibitors are described in, for example, WO 2020/200880, which is incorporated herein by reference.

Although topoisomerase I inhibitors are preferred as outlined above, it should be noted that any suitable ADC payload (e.g., drug/cytotoxin) may be linked to an ADC antibody or antigen-binding fragment thereof.

In certain embodiments, the anti-FRα ADC used in the invention does not comprise a microtubule inhibitor such as a tubulin inhibitor (e.g., maytansinoids, auristatins). The microtubule inhibitor class of molecules suffers from potentially difficult-to-treat toxicities that limit dosing.

### VI. Preparation of ADC

The ADCs of the present disclosure can be made in a variety of ways, using known organic chemistry reactions, conditions, and reagents, such as: (1) reacting a reactive substituent of an ADC antibody or antigen-binding fragment with a bivalent linker reagent, then reacting with an ADC payload (e.g., cytotoxin), preferably topoisomerase I inhibitor; or (2) reacting a reactive substituent of an ADC payload (e.g., cytotoxin), preferably topoisomerase I inhibitor, with a bivalent linker reagent, then reacting with a reactive substituent of an ADC antibody or antigen-binding fragment thereof.

Reactive substituents that may be present within an ADC antibody, or antigen-binding fragment thereof, as disclosed herein include, without limitation, nucleophilic groups such as (i) N-terminal amine groups, (ii) side chain amine groups, e.g., lysine, (iii) side chain thiol groups, e.g., cysteine, and (iv) sugar hydroxyl or amino groups where the antibody is glycosylated. Reactive substituents that may be present within an ADC antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an ADC antibody, or antigen-binding fragment thereof as disclosed herein include amine or thiol moieties. Certain antibodies have cysteine bridges, which are reducible interchain disulphides. By treating antibodies with a reducing agent (such as DL-dithiothreitol (DTT) and tris(2-carboxyethyl)phosphine (TCEP)), they can be made reactive for conjugation with linker reagents. Each cysteine bridge will theoretically result in the formation of two reactive thiol nucleophiles. The reaction of lysines with 2-iminothiolane (Traut's reagent), which results in the conversion of an amine to a thiol, can be used to introduce additional nucleophilic groups into antibodies. One, two, three, four, or more cysteine residues can be used to insert reactive thiol groups into an ADC antibody (or fragment thereof) (e.g., preparing mutant antibodies comprising one or more non-native cysteine amino acid residues). Engineering antibodies with reactive cysteine amino acids is described in U.S. Pat. No. 7,521,541, which is incorporated herein by reference.

In some embodiments, the ADC antibody or antigen-binding fragment thereof may have one or more carbohydrate groups that can be chemically changed to contain one or more sulfydryl groups. The ADC is then formed by conjugation through the sulfur atom of the sulfydryl group.

In some embodiments, the ADC antibody may contain one or more carbohydrate groups that can be oxidized to produce an aldehyde (—CHO) group (see, for example, Laguzza et al., J. Med. Chem. 1989, 32(3), 548-55). Conjugation through the corresponding aldehyde results in the formation of the ADC. Further protocols for the modification of proteins for the attachment or association of ADC payloads (e.g., cytotoxins) are described in Coligan et al., Current Protocols in Protein Science, vol. 2, John Wiley & Sons (2002). Methods for the conjugation of linker-drug moieties to cell-targeted proteins such as antibodies, immunoglobulins or fragments thereof are found, for example, in U.S. Pat. Nos. 5,208,020; 6,441,163; WO2005/037992; WO2005/081711; and WO2006/034488, each of which is incorporated herein by reference.

Conventional conjugation strategies for antibodies or antigen-binding fragments thereof rely on randomly or stochastically conjugating the payload to the antibody or fragment through lysines or cysteines. In some embodiments, the ADC antibody or antigen-binding fragment thereof is stochastically conjugated to an ADC payload (e.g., cytotoxin), preferably topoisomerase I inhibitor, for example, by partial reduction of the ADC antibody or fragment, followed by reaction with a desired ADC payload, with or without a linker moiety attached. The ADC antibody or fragment may be reduced using DTT or other reducing agent to perform a similar reduction e.g., TCEP. The ADC payload with or without a linker moiety attached can then be added at a molar excess to the reduced antibody or fragment in the presence of DMSO. After conjugation, a quenching agent such as N-acetyl-L-cysteine may be added to quench unreacted agent. The reaction mixture may then be purified (by e.g., TFF, SEC-FPLC, CHT, spin filter centrifugation) and buffer-exchanged into PBS or other relevant formulation buffer.

In some embodiments, an ADC payload (e.g., cytotoxin) is conjugated to an ADC antibody or antigen-binding fragment thereof by site-specific conjugation. In some embodiments, site-specific conjugation of therapeutic moieties to ADC antibodies using reactive amino acid residues at specific positions yields homogeneous preparations of an ADC with uniform stoichiometry.

The site-specific conjugation can be through a cysteine, residue or a non-natural amino acid. In a preferable embodiment, the ADC payload (e.g., cytotoxin) is conjugated to the ADC antibody or antigen-binding fragment thereof through at least one cysteine residue. Cysteine amino acids may be engineered at reactive sites in an ADC antibody (or antigen-binding fragment thereof) and preferably do not form intrachain or intermolecular disulfide linkages (Junutula, et al., 2008b Nature Biotech., 26(8):925-932; Dornan et al. (2009) Blood 114(13):2721-2729; U.S. Pat. Nos. 7,521,541; 7,723,485; WO2009/052249). In some embodiments, the ADC payload (e.g., cytotoxin) is conjugated to the antibody or antigen-binding fragment thereof through a cysteine substitution of at least one of positions 239, 248, 254, 273, 279, 282, 284, 286, 287, 289, 297, 298, 312, 324, 326, 330, 335, 337, 339, 350, 355, 356, 359, 360, 361, 375, 383, 384, 389, 398, 400, 413, 415, 418, 422, 440, 441, 442, 443 and 446, wherein the numbering corresponds to the EU index in Kabat. In some embodiments, the specific Kabat positions are 239, 442, or both. In some embodiments, the specific positions are Kabat position 442, an amino acid insertion between Kabat positions 239 and 240, or both. In some embodiments, the ADC payload (e.g., cytotoxin) is conjugated to the ADC antibody or antigen-binding fragment thereof through a thiol-maleimide linkage. In some aspects, the amino acid side chain is a sulfydryl side chain.

Where more than one nucleophilic or electrophilic group of the ADC antibody or antigen-binding fragment thereof reacts with an ADC payload (e.g., cytotoxin), then the resulting product may be a mixture of ADCs with a distribution of ADC payload units attached to an antibody, e.g., 1, 2, 3, etc. Liquid chromatography methods such as hydrophobic interaction (HIC) may separate compounds in the mixture by ADC payload loading value. Preparations of an ADC with a single ADC payload loading value (p) may be isolated.

The average number of ADC payloads (e.g., cytotoxins) per ADC antibody (or antigen-binding fragment) in preparations of ADCs from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of an ADC may be determined (Hamblett et al. (2004) Clin. Cancer Res. 10:7063-7070; Sanderson et al. (2005) Clin. Cancer Res. 11:843-852). In some instances, separation, purification, and characterization of homogeneous ADC, where p is a certain value from antibody with other ADC payloads (e.g., cytotoxins), may be achieved by means such as reverse phase HPLC, electrophoresis, TFF, SEC-FPLC, CHT, spin filter centrifugation. Such techniques are also applicable to other types of conjugates.

### VII. Pharmaceutical Compositions

The anti-FRα ADC may be administered as a pharmaceutical composition containing one or more pharmaceutically compatible components.

The term “pharmaceutical composition” refers to a preparation that is in such form as to permit the biological activity of the active ingredient to be effective and that contains no additional components which are unacceptably toxic to a subject to which the composition would be administered. Such composition can be sterile, and can comprise a pharmaceutically acceptable carrier, such as physiological saline. Suitable pharmaceutical compositions can comprise one or more of a buffer (e.g., acetate, phosphate or citrate buffer), a surfactant (e.g., polysorbate), a stabilizing agent (e.g., human albumin), a preservative (e.g., benzyl alcohol), and absorption promoter to enhance bioavailability, and/or other conventional solubilizing or dispersing agents.

The term “pharmaceutically acceptable” as used herein means approved by a regulatory agency of a federal or state government, or listed in the U.S. Pharmacopeia, European Pharmacopeia or other generally recognized pharmacopeia for use in animals, and more particularly in humans.

In some embodiments, the pharmaceutical composition may be comprised within one or more formulations selected from a capsule, a tablet, an aqueous suspension, a solution, a nasal aerosol, a lyophilized powder that can be reconstituted to make a suspension or solution before use, or a combination thereof.

For example, the anti-FRα ADC AB1370049-SG3932-DAR8 used in the novel methods may be administered as a pharmaceutical composition containing a buffering agent such as a histidine buffering agent, an excipient such as sucrose, and a surfactant such as polysorbate 80. The pharmaceutical composition containing the anti-FRα ADC used in the novel methods can be used as an injection, as an aqueous injection or a lyophilized injection, or even as a lyophilized injection.

The pharmaceutical compositions disclosed herein can be administered to a patient by any appropriate systemic or local route of administration. For example, administration may be oral, buccal, sublingual, ophthalmic, intranasal, intratracheal, pulmonary, topical, transdermal, urogenital, rectal, subcutaneous, intravenous, intra-arterial, intraperitoneal, intramuscular, intracranial, intrathecal, epidural, intraventricular or intratumoral.

The pharmaceutical compositions disclosed herein can be formulated for administration by any appropriate means, for example, by epidermal or transdermal patches, ointments, lotions, creams, or gels; by nebulizers, vaporizers, or inhalers; by injection or infusion; or in the form of capsules, tablets, liquid solutions or suspensions in water or non-aqueous media, drops, suppositories, enemas, sprays, or powders. The most suitable route for administration in any given case will depend on the physical and mental condition of the patient, the nature and severity of the disease, and the desired properties of the formulation.

Pharmaceutical compositions for oral administration may be in tablet, capsule, powder or liquid form. A tablet may comprise a solid carrier or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution that is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

If the pharmaceutical composition containing the anti-FRα ADC used in the novel methods is an aqueous injection, it can be diluted with a suitable diluent and then given as an intravenous infusion. Examples of the diluent can include dextrose solution and physiological saline.

If the pharmaceutical composition containing the anti-FRα ADC used in the novel methods is a lyophilized injection, it can be dissolved with injection-grade water, then diluted for a requisite amount with a suitable diluent and then given as an intravenous infusion. Examples of the diluent include dextrose solution and physiological saline.

### VIII. Therapeutic Uses of the Anti-FRα ADC

Preferably, the ADC disclosed herein can be used to prevent, treat, or ameliorating symptoms associated with a disease, disorder, or infection (preferably cancer). The term “cancer” is used to have the same meaning as that of the term “tumor”.

To “treat” refers to therapeutic measures that cure, slow down, alleviate symptoms of, and/or halt progression of a diagnosed pathologic condition or disorder. Thus, those in need of treatment include those already with the disorder. In some embodiments, a subject is successfully “treated” for a disease or disorder (preferably cancer), according to the novel methods disclosed herein if the patient shows, e.g., total, partial, or transient alleviation or elimination of symptoms associated with the disease or disorder (preferably cancer).

To “prevent” refers to prophylactic or preventative measures that prevent and/or slow the development of a targeted pathologic condition or disorder (preferably cancer). Thus, those in need of prevention include those prone to have or susceptible to the disorder.

The terms “patient”, “individual” and “subject” are used interchangeably herein to refer to a mammalian subject. In some embodiments, the “subject” is a human, domestic animals, farm animals, sports animals, and zoo animals, e.g., humans, non-human primates, dogs, cats, guinea pigs, rabbits, rats, mice, horses, cattle, etc. In some embodiments, the subject is a cynomolgus monkey (Macaca fascicularis). In a preferable embodiment, the subject is a human. In some embodiments of the novel methods, the patient might not have been previously diagnosed as having cancer. Alternatively, the patient may have been previously diagnosed as having cancer. The patient may also be one who exhibits disease risk factors, or one who is asymptomatic for cancer. The patient may also be one who is suffering from or is at risk of developing cancer. Thus, in one embodiment, a method of the invention may be used to confirm the presence of cancer in a patient. For example, the patient may previously have been diagnosed with cancer by alternative means. In one embodiment, the patient has been previously administered a cancer therapy.

In some embodiments, the anti-FRα ADC disclosed herein may be used for treating a cancer associated with FRα expression. In other words, a cancer referred to herein may comprise a cancerous cell that expresses FRα. Said cancerous cell may be comprised within a tumor. In some embodiments, the cancer includes cancer cells with heterogeneous expression of FRα and/or a low expression of FRα.

After undergoing cellular internalization into cancer cells, the anti-FRα ADC used in the present invention is cleaved at the Linker unit and releases the Drug unit into the cancer cell. The anti-FRα antibody-drug conjugate used in the novel methods also has a bystander effect in which the anti-FRα antibody-drug conjugate is internalized into cancer cells that express the target protein FRα, and the Drug unit then also exerts an antitumor effect on neighboring cancer cells that do not express the target protein FRα.

Preferably, the cancer is selected from ovarian cancer, lung cancer, endometrial cancer, breast cancer (e.g., TNBC), cervical cancer, pancreatic cancer, gastric cancer, renal cell carcinoma (RCC), colorectal cancer, head and neck squamous cell carcinomas (HNSCC) and malignant pleural mesothelioma. More preferably, the cancer is ovarian cancer or lung cancer. In some embodiments, the cancer is one or more of non-small-cell lung carcinoma (NSCLC) preferably selected from squamous NSCLC, adenocarcinoma NSCLC, or a combination thereof.

Further examples of cancer include, but are not limited to, benign, pre-malignant, and malignant cellular proliferation, including but not limited to, neoplasms and tumors (e.g., histocytoma, glioma, astrocyoma, osteoma), cancers (e.g. ovarian carcinoma, lung cancer, non-small cell lung cancer (squamous cell carcinoma or adenocarcinoma), endometrial cancer, pancreatic cancer, gastric cancer, colorectal cancer, head and neck squamous cell carcinomas, malignant pleural mesothelioma, breast carcinoma (e.g. TNBC), and kidney cancer. Any type of cell may be treated, including but not limited to, lung, gastrointestinal, breast (mammary), ovarian, kidney (renal), and pancreas.

The anti-FRα ADC disclosed herein can be expected to exert a therapeutic effect by application as systemic therapy to patients, and additionally, by local application to cancer tissues.

### IX. Diagnostic Antibody

The diagnostic antibody used in the novel methods binds to FRα on the cells (e.g., cancer cells) in a tissue sample of the cancer patient. In preferred embodiments, the diagnostic antibody specifically binds to human FRα. The diagnostic antibody is labeled to aid detection of cell binding. In some embodiments, the label may be a fluorophore or a dye. In some embodiments, the label could be used for immunohistochemistry. In a preferred embodiment, the diagnostic antibody is linked to 3,3′-Diaminobenzidine (DAB). In some embodiments, the diagnostic antibody is an anti-FRα antibody obtained from Ventana (VMSI, #742-5065). In some embodiments, the diagnostic antibody is an anti-rabbit FRα antibody. In a preferred embodiment, the diagnostic antibody is an anti-rabbit FRα antibody obtained from Abcam (#ab221543). In some embodiments, the diagnostic antibody is closely associated with the ADC antibody or antigen-binding fragment as described herein. In some embodiments, the diagnostic antibody is closely associated with the ADC antibody of the ADC AB1370049-SG3932.

In some embodiments, the diagnostic antibody binds to cancer cells from ovarian cancer, lung cancer, endometrial cancer, colon cancer, breast cancer (e.g., TNBC), cervical cancer, pancreatic cancer, gastric cancer, renal cell carcinoma (RCC), colorectal cancer, head and neck squamous cell carcinomas (HNSCC) and malignant pleural mesothelioma. More preferably, the diagnostic antibody binds to cancer cells from ovarian cancer, lung cancer, endometrial cancer or breast cancer. In some embodiments, the diagnostic antibody binds to cancer cells from non-small-cell lung carcinoma (NSCLC), preferably selected from squamous NSCLC, adenocarcinoma NSCLC, or a combination thereof.

### X. Method of Predicting a Patient Response to Administering an Anti-FRα ADC

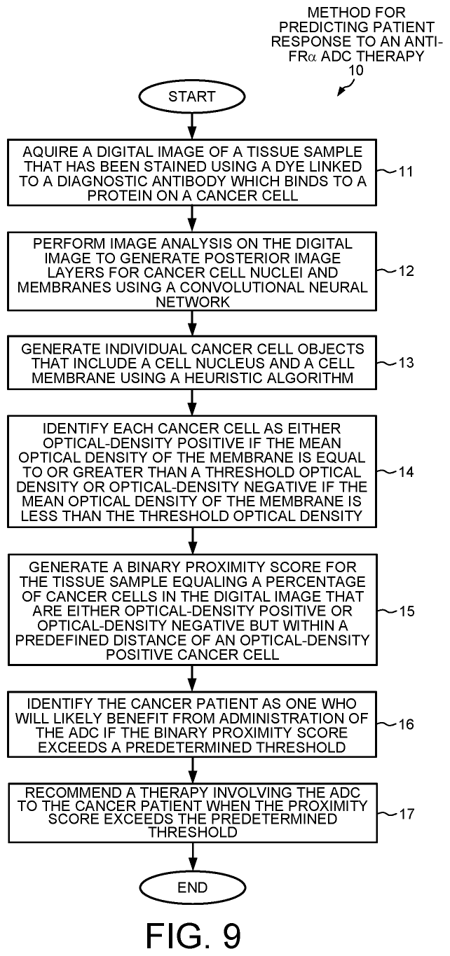

FIG. 9 is a flowchart of steps 11-17 of a method 10 by which an analysis system analyzes a digital image of tissue from a cancer patient and predicts how the cancer patient will likely respond to a therapy involving an anti-FRα antibody-drug conjugate (ADC). In one embodiment, the ADC is the anti-FRα ADC AB1370049-SG3932-DAR8. In one embodiment, the method predicts the response to an ADC of a patient having a cancer selected from ovarian cancer, lung cancer, endometrial cancer, breast cancer (e.g., TNBC), cervical cancer, pancreatic cancer, gastric cancer, renal cell carcinoma (RCC), colorectal cancer, head and neck squamous cell carcinomas (HNSCC) and malignant pleural mesothelioma. More preferably, the method predicts the response to an ADC of a patient having a cancer selected from ovarian cancer, non-small-cell lung cancer, endometrial cancer and colon cancer. In some embodiments, the method predicts the response to an ADC of a patient with non-small-cell lung carcinoma (NSCLC) preferably selected from squamous NSCLC, adenocarcinoma NSCLC, or a combination thereof.

In a first step 11, a high-resolution digital image is acquired of a tissue slice from the cancer patient that has been stained using one or more biomarkers or stains.

To predict the efficacy of the ADC therapy, a diagnostic antibody (e.g., a diagnostic biomarker) with an attached dye is used that targets the same protein as that targeted by the ADC therapy. In one embodiment, the anti-FRα ADC therapy to which the scoring is directed is an anti-FRα antibody conjugated to a topoisomerase I inhibitor.

Thus, in embodiments, the diagnostic antibody also targets the FRα protein.

In step 12, a pretrained convolutional neural network processes a digital image of tissue of the cancer patient that has been stained with the diagnostic antibody linked to the dye, such as 3,3′-Diaminobenzidine (DAB). The staining intensity of the dye in the membrane of a cancer cell is determined based on the mean staining intensity of the dye of all pixels associated with the corresponding segmented membrane object. Moreover, the staining intensity of the dye in a single pixel is computed based on the red, green and blue color components of the pixel. The result of the image analysis processing is two posterior image layers representing, for each pixel in the digital image, the probability that the pixel belongs to a cell nucleus and the probability that the pixel belongs to a cell membrane.

In step 13, individual cancer cells are detected based on a heuristic image analysis of the posterior layers for nuclei and membranes. Cancer cell objects are generated that include cell membrane objects.

In step 14, each cancer cell is identified as being either optical-density positive or optical-density negative based on the amount of DAB in the cell membrane. The amount of DAB is determined by the staining intensity of each membrane based on the mean (average) optical density of the brown diaminobenzidine (DAB) signal in all of the pixels of the membrane. Each cancer cell is identified as either (i) optical-density positive if the mean optical density of the cancer cell is equal to or greater than an optical density threshold, or (ii) optical-density negative if the mean optical density of the cancer cell is less than the optical density threshold. In one embodiment, the optical density threshold is in a range of ten to fifteen on a scale with a maximum optical density of 220. For example, the optical density threshold is twelve.

In step 15, a binary proximity score for the digital image of the tissue sample is generated equaling the percentage of cancer cells in the digital image that are either optical-density positive or optical-density negative but within a predefined distance of an optical-density positive cancer cell. In one embodiment, the predefined distance is twenty-five microns.

In step 16, the cancer patient is identified as one who will likely benefit from administration of the anti-FRα ADC if the proximity score exceeds a predetermined percentage threshold. In one embodiment, the predetermined percentage threshold is twenty.

The threshold optical density used in step 14, the predefined distance used in step 15, and the predetermined percentage threshold used in step 16 are optimized using a training cohort of patients with known responses to the ADC therapy. Optimization is performed using Spearman correlation analysis of the proximity score versus response of patients to obtain the lowest p values and Rank R values. Alternatively, receive operating characteristic (ROC) analysis is performed for cutpoint optimization to distinguish binary clinical outcome (e.g., responsive R/non-responsive NR). The binary proximity score is indicative of how the cancer patient will respond to a therapy involving an anti-FRα ADC.

In step 17, the therapy involving the anti-FRα ADC is recommended to score-positive patients if the score exceeds the predetermined percentage threshold.

### XI. Examples of Prediction and Scoring Method

### A. Image Analysis of Stained Tissue

The method of FIG. 9 is now described in relation to a particular image of stained cancer tissue.

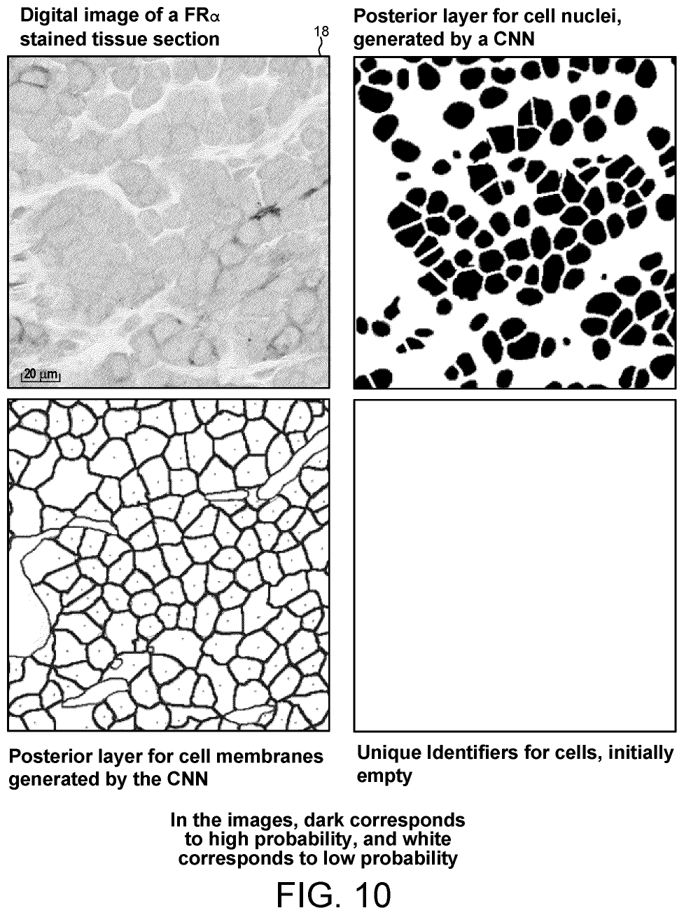

In step 11, a tissue sample is immunohistochemically stained using a dye linked to a diagnostic antibody that binds to the associated protein on the cancer cells in the tissue sample. FIG. 10 (upper-left image) is a digital image 18 of a portion of stained tissue that was acquired in step 11. Image 18 shows tissue from a cancer patient that has been immunohistochemically stained with an anti-FRα diagnostic antibody linked to a dye. In this example, the diagnostic antibody is an anti-FRα generated clone (denoted FRα IHC Tool Ab) reformatted as a mouse anti-human FRα IgG1 clone. IgG1 indicates the isotype of the anti-FRα antibody. The staining protocol was developed using the Dako Autostainer platform. The anti-FRα antibody binds to the membrane protein FRα so that the 3,3′-Diaminobenzidine (DAB) stain indicates the location of the protein FRα in the tissue sample.

In step 12, image analysis is performed on the digital image 18 to generate posterior image layers of cancer cell nuclei and membranes using a convolutional neural network. The image analysis is used to detect the cancer cells and their components, such as the nuclei, the membrane and the cytoplasm. FIG. 10 illustrates the image analysis process of step 12. The convolutional neural network generates posterior layers (gray value images) that indicate, for each pixel of digital image 18, the probability that each pixel belongs to either the nucleus (FIG. 10 upper-right image) or the membrane (FIG. 10 lower-left image) of the cell. High probabilities are shown in black, low probabilities in white. The lower-left image also shows the nuclei centers that are first identified in order to generate the cell membrane objects by growing outwards from the nuclei centers until meeting growth from another nucleus center.

In one embodiment, the convolutional neural network includes a series of convolution layers from the input image 18 towards a bottleneck layer with very low spatial size (1 to 16 pixels), and a series of deconvolution layers towards the posterior layers that have the same size as the input image 18. This network architecture is called a U-Net. The training of the weights of the convolutional neural networks is performed by generating manual annotation layers for nuclei and membranes in multiple training images, and then adjusting by an optimization algorithm the network weights so that the generated posterior layers are most similar to the manually generated annotation layers.

In another embodiment, the annotation layers for nuclei and membranes are generated automatically and corrected manually in multiple training images. Epithelium regions and nuclei centers are manually annotated as regions and points, respectively. For each training image, the membrane segmentation is automatically generated by applying a region growing-like algorithm (e.g., watershed segmentation) seeded by the annotated nuclei centers and constrained by the extent of the annotated epithelium region. Given a training image, the nuclei segmentation is automatically generated by applying a blob detection algorithm (e.g., by the maximally-stable-extremal-regions MSER algorithm) and by selecting as nuclei only the detected blobs that contain an annotated nucleus center. The automatically generated membrane and nuclei segmentations are visually reviewed and manually corrected if necessary. The correction steps involve one of the following methods: rejecting incorrectly segmented membranes or nuclei, explicitly accepting correctly annotated membranes or nuclei, or refining the shapes of the membranes or nuclei. For each image with annotated membranes or nuclei, an annotation layer is created. In one embodiment, each pixel of the annotation layer is assigned a “1” if it belongs to the annotated object (membrane or nucleus); otherwise it is assigned a “0”. In another embodiment, the pixels of the annotation layer represent the distance to the nearest annotated object. The network weights are adjusted by an optimization algorithm so that the generated posterior layers are most similar to the automatically generated membrane and nuclei annotation layers.

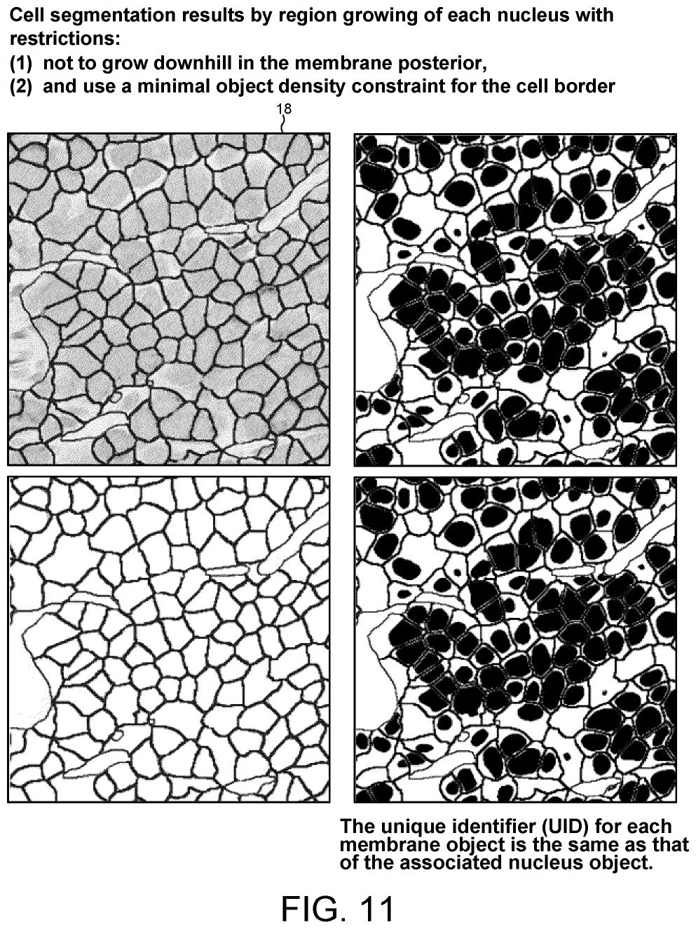

FIG. 11 illustrates step 13 in which individual cancer cell objects are detected that each include a cell membrane and a cell cytoplasm. A heuristic image analysis process uses watershed segmentation to segment the cell nuclei using the nucleus posterior layer generated by the convolutional neural network. The segmentation generates nucleus objects. Each nucleus object is assigned a unique identifier (UID). The individually identified nuclei are shown as dark objects in FIG. 11 (lower-right image). The detected nuclei are also displayed as overlays in the input image 18 (upper-left image) and in the posterior layers for nuclei (upper-right image) and membranes (lower-left image).

In one embodiment, the watershed segmentation involves a thresholding of the nucleus posterior layer with a predefined first size threshold. All single connected pixels that are above a first size threshold are considered to belong to a nucleus object. Nucleus objects with an area smaller than 16 um\hat{ }2 are discarded. A UID is assigned to each nucleus object. In a subsequent step, the nucleus objects are grown towards smaller nucleus posteriors in which the added nucleus posterior pixels must be greater than a second predefined threshold.

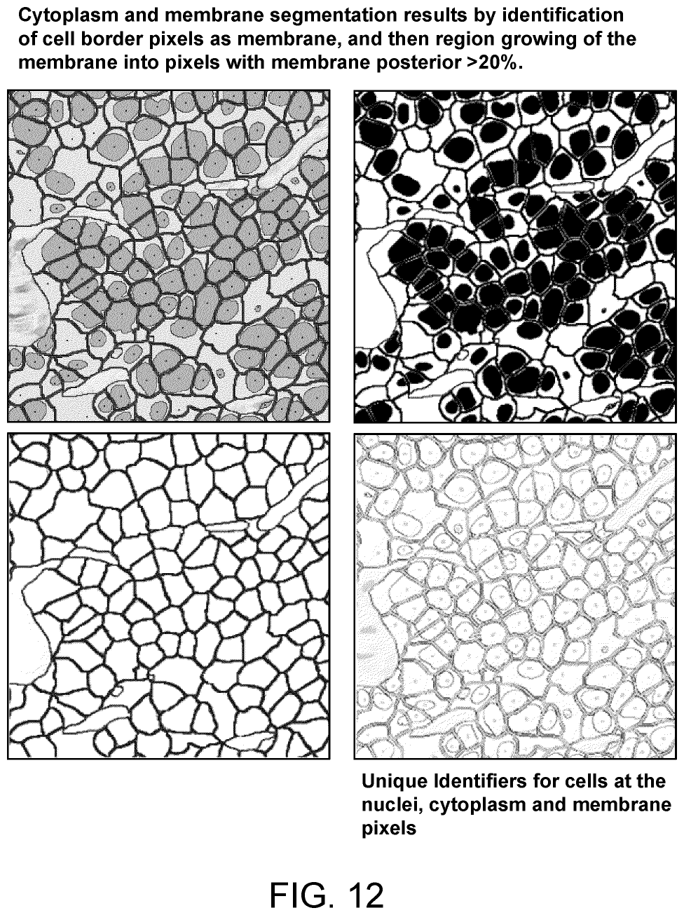

FIG. 12 illustrates the detection and segmentation of membrane objects which are segmented by growing the region of the border pixels of detected cells outwards to the membrane probability layer and to a predefined membrane layer posterior threshold value. The thicker border regions become the membrane objects. Each membrane object is assigned the same UID as that of the associated nucleus object.

The space between the membrane and the nucleus is assigned to the cytoplasm using the UID of the nucleus. For each membrane (FIG. 12 see top-left image) and cytoplasm (FIG. 12 see top-left image), the average optical densities of the DAB staining is exported to a file on a hard drive together with the UIDs. For each cell (defined as including a nucleus, cytoplasm and membrane), the position of the center of gravity (x,y) of the cell within the slide is also exported. The file may reside on a hard disk, a solid state disk or a portion of dedicated RAM in a computer system.

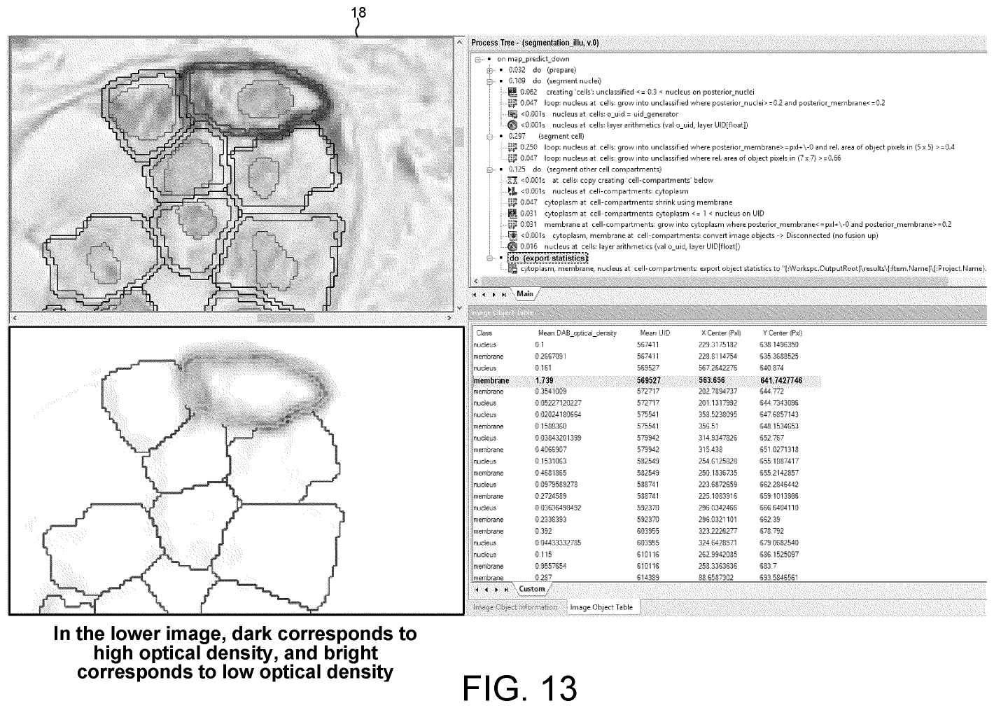

FIG. 13 illustrates the results of the image analysis in an image analysis software environment. FIG. 13 (upper-left image) shows the segmentation of nucleus objects and membrane objects as an overlay on a digital image of stained tissue. FIG. 13 (lower-left image) shows the segmentation of a nucleus object as an overlay on an optical density representation of the digital image. Dark optical density pixels are associated with a high amount of DAB, and bright optical density pixels are associated with a low amount of DAB. The DAB optical density of each image pixel is computed from the red-green-blue representation of the image pixel by transformation of the red-green-blue color space so that the brown DAB component becomes an independent color, and by taking the logarithm of that brown color component. FIG. 13 (upper-right image) shows the image analysis script used to generate the segmented image. FIG. 13 (lower-right image) shows the exported measurements for all cell membrane objects and cytoplasm objects in image 18.

In another embodiment the cell membrane of each tumor cell is detected by a single shot instance segmentation method “StarDist” as described in [https://squidpy.readthedocs.io/en/stable/external_tutorials/tutorial_stardist.html]. This method approximates the cell membrane as a closed polygon with N star-like support vectors originating from the cell center. The network predicts N+1 posterior layers whereas the layer N+1 associates each pixel value with the probability that this pixel belongs to a cell center. In each of the N posterior layers the distance of the membrane to the cell center pixel is predicted. The mean optical density for each cell membrane is computed by iterating the predicted closed polygon and measuring the optical densities of the associated pixels crossed by the polygon.

### B. Calculation of a Predictive Efficacy Score

Based on the optical density of the DAB staining within the membrane objects and optionally the cytoplasm objects, a spatial proximity score is determined for the tissue sample shown in the digital image 18. The spatial proximity score also accounts for the staining intensities of the DAB dye in the membrane objects and optionally cytoplasm objects of neighboring cancer cells that are closer than a predefined distance to the cancer cell for which the single-cell ADC score is being computed. There are two types of spatial proximity scores: a binary spatial proximity score (bSPS) and a continuous spatial proximity score (cSPS).

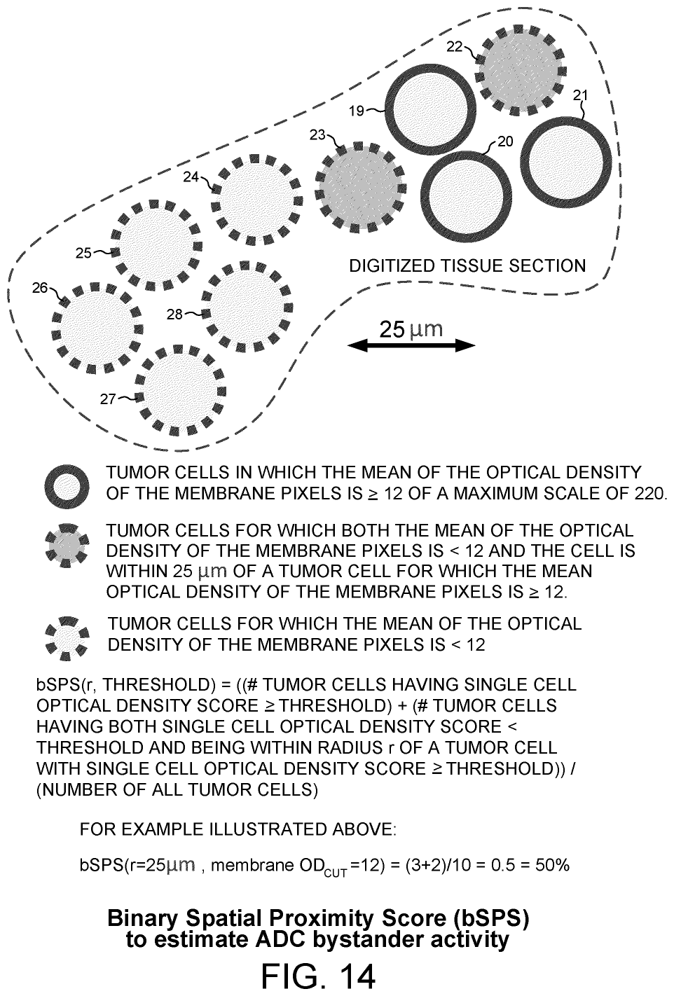

FIG. 14 shows an example of how the binary spatial proximity score (bSPS) is determined. The binary spatial proximity score is a percentage score equal to the sum of (i) the number of cancer cells in the digital image 18 whose staining intensity equals or exceeds an optical density threshold plus (ii) the number of cancer cells in the digital image whose staining intensity is less than the optical density threshold but that are within a predefined distance of at least one cancer cell whose staining intensity equals or exceeds the optical density threshold, the sum divided by the total number of cancer cells in the digital image 18.

FIG. 14 illustrates ten exemplary cancer cells (also called tumor cells) with various optical densities of staining from the image analysis of steps 12-13. The steps of heuristic image analysis illustrated in FIGS. 10-12 are used to obtain the segmentation into image objects including cell membrane objects and cancer cell objects. Circles with solid lines around the circumference represent tumor cells whose optical density of brown DAB staining (corresponding to the amount of target protein FRα) is greater than or equal to a predetermined optical density threshold. In this example, the optical density threshold is 12 from a maximum scale of optical density of 220. Three cancer cells 19-21 in FIG. 14 are classified as optical-density positive. Circles with dashed circumferences represent tumor cells whose optical density of brown DAB staining is less than the predetermined optical density threshold. Seven cancer cells 22-28 are classified as optical-density negative. Darker gray circles with dashed circumferences represent optical-density negative cancer cells that are located within a predefined distance of at least one optical-density positive cancer cell. The two cancer cells 22-23 are optical-density negative as well as disposed within the predefined distance from an optical-density positive cancer cell. In this example, the predefined distance is 25 microns.

In this example, the binary spatial proximity score equals the percentage score of 50%, which is calculated as shown in FIG. 14 as the sum of three optical-density positive cells and two optical-density negative that are within the predefined distance of an optical-density positive cell, the sum divided by the total of ten cancer cells ([3+2]/10=0.5).

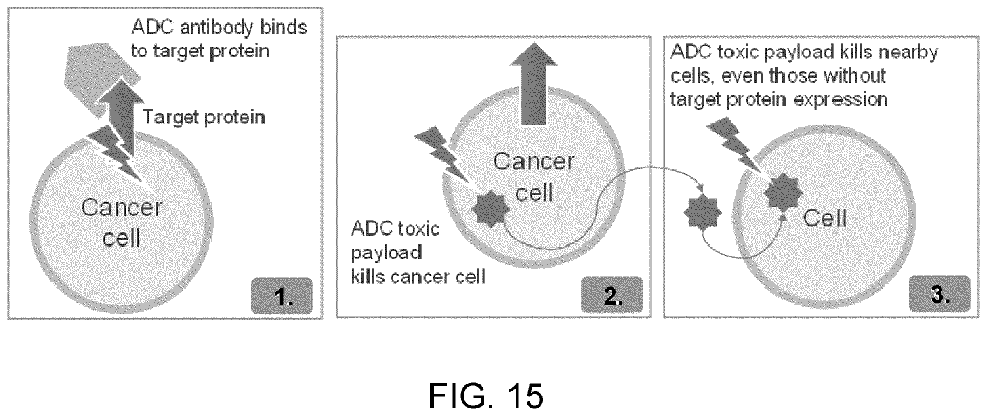

In the schematic image of FIG. 14, the three cancer cells 19-21 express a high amount of the target protein FRα and would very likely be killed by the ADC payload (e.g., cytotoxin) entering the cell linked to the ADC antibody (effect 1 in FIG. 15). The two cancer cells 22-23 do not express sufficient amounts of the target protein FRα to be killed directly by the anti-FRα ADC. However, due to the proximity of at least one of the optical-density positive cells 19-21, the toxic payload released from cells 19-21 would also kill the cancer cells 22-23 (effect 3 in FIG. 15). The remaining optical-density negative cancer cells 24-28 would remain active and could be the origin of a drug resistance mechanism, which could eventually cause the death of the patient.

FIG. 15 illustrates the mechanism by which an anti-FRα ADC therapy kills cancer cells. In a first step, the ADC antibody binds to the target protein FRα and inhibits the natural function of the target protein, which may lead to cell death. In a second step, the payload (e.g., a type I topoisomerase inhibitor) is internalized into the cell and kills the cell by the toxicity of the payload. This uptake of the payload depends on the amount of target protein on the membrane, and also on the difference in the amount of target protein on the membrane and in the cytoplasm. After uptake, the payload can be released from the cell into the surrounding tissue. In a third step, the payload may enter nearby cells and may kill them as well. The spatial distribution of the payload in the tissue is spread by passive diffusion.

Traditional IHC scoring reflects both the effect of inhibition of the target protein due to ADC binding, as well as the effect of the cytotoxic payload entering a cancer cell together with the ADC antibody. Thus, the traditional scoring for ADC therapies does not reflect the importance of the presence of the target protein in the cytoplasm and the effect of the cytotoxic payload that diffuses into the tissue after being released from the first killed cancer cell. In comparison, the novel predictive spatial proximity score measures the effect of the release of the cytotoxic payload on neighboring cancer cells.

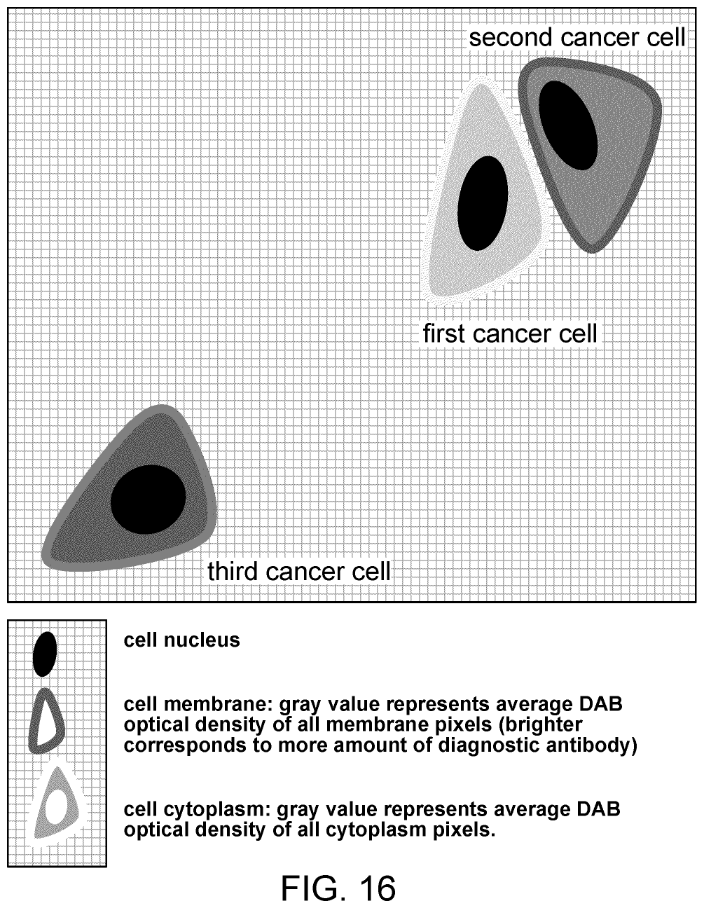

FIGS. 16-18 illustrate an example of how the continuous spatial proximity score is determined. Although the continuous spatial proximity score applies a predefined distance, optical-density negative cancer cells are still attributed some weight despite being disposed farther than the predefined distance from an optical-density positive cancer cell. In addition, this embodiment of the continuous spatial proximity score also takes into account staining in the cytoplasm. The continuous spatial proximity score is computed for the tissue sample shown in the digital image 18 based on the optical density of the DAB staining within the membrane objects and optionally the cytoplasm objects.

FIG. 16 illustrates exemplary quantitative results of the optical density of staining from the image analysis of steps 12-13 in a schematic drawing using gray values of membrane and cytoplasm pixels. The steps of heuristic image analysis illustrated in FIGS. 10-12 are used to obtain the example segmentation of FIG. 16 into cell nuclei, cell membranes and cell cytoplasm. Bright gray values in FIG. 16 are associated with high DAB optical density, and therefore with a high amount of proteins targeted by the diagnostic antibody. Dark gray values are associated with a low DAB optical density. Brighter pixels correspond to a higher DAB optical density.

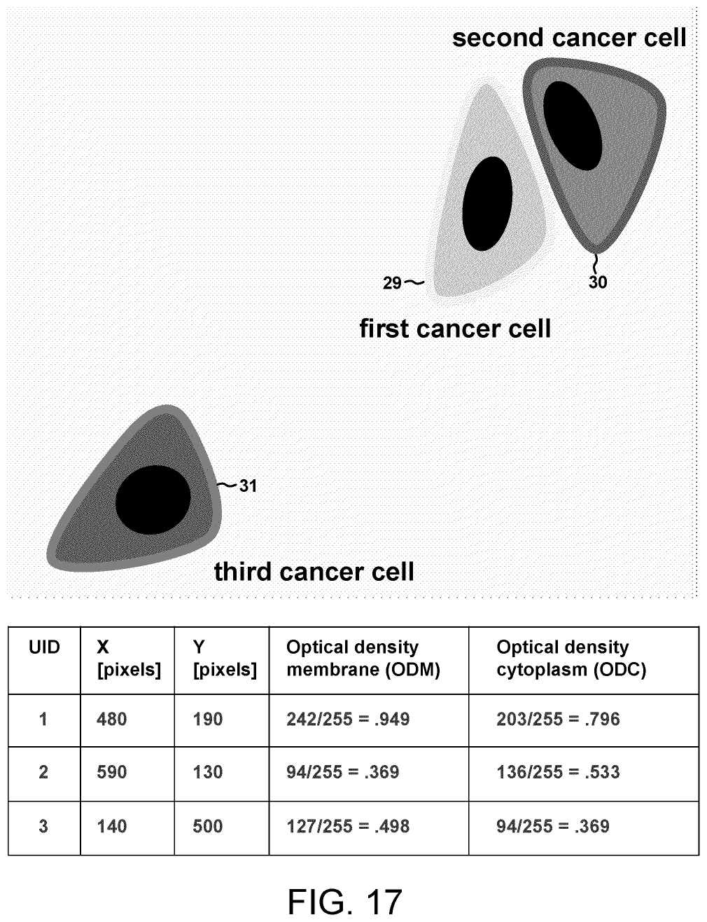

FIG. 17 lists the exemplary quantitative amounts of staining on the membranes and in the cytoplasms of the image of FIG. 16, which is reproduced in part in FIG. 17. The optical density of the brown DAB signal from the membranes of the first, second and third cells is 0.949, 0.369 and 0.498, respectively. In this example, the optical density is expressed as a percentage of the maximum staining of 255. The optical density of the brown DAB signal from the cytoplasms of the first, second and third cells is 0.796, 0.533 and 0.369, respectively. In the schematic image of FIG. 17, the first cancer cell 29 expresses a high amount of the target protein FRα and would be very likely to be killed by the ADC payload (e.g., cytotoxin) entering the cell linked to the ADC antibody (effect 1 in FIG. 15). The second cancer cell 30 and third cancer cell 31 do not express sufficient amounts of the target protein FRα to be killed directly by the anti-FRα ADC. However, due to the vicinity of the second cancer cell 30 to the first cancer cell 29, the toxic payload released from the first cancer cell would also kill the second cancer cell 30 (effect 3 in FIG. 15). The third cancer cell 31 would remain active and could be the origin of a drug resistance mechanism, which could eventually cause the death of the patient.

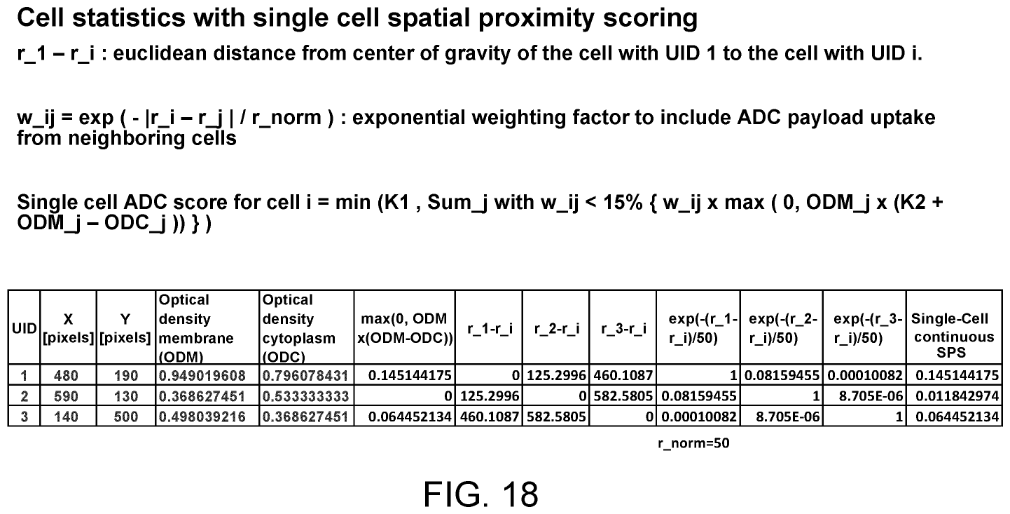

In step 14 of the method of FIG. 9, the mean optical density of each cancer cell is determined. Then in a modification of method 10, a single-cell spatial proximity score is determined. FIG. 18 illustrates the calculation of the single-cell spatial proximity score for each of the three cells shown in FIG. 17 and incorporates an exponential weighting factor for cell separation to reflect the uptake of the ADC payload (e.g., cytotoxin) into neighboring cells. The single-cell score can be calculated based on the formula shown in FIG. 18. The predefined distance used in the formula is 50 microns. The optical densities listed in FIG. 17 for the DAB signal from the cell membranes (0.949, 0.369 and 0.498) and from the cytoplasms (0.796, 0.533 and 0.369) are inputs into the calculation illustrated in FIG. 18. The first, second and third cells 29, 30, 31 have single-cell spatial proximity scores of 0.145, 0.012 and 0.064, respectively.

Thus, the continuous spatial proximity score incorporates the measurement of the amount of target protein on the cell membrane using the DAB optical density and optionally an estimation of the amount of ADC payload (e.g., cytotoxin) uptake. As shown in FIG. 17, the uptake of the ADC payload (e.g., cytotoxin) for a first cell depends on both the amount of dye in its membrane and in its cytoplasm, as well as on the amount of dye in the membrane and the cytoplasm of a second cell in the vicinity of the first cell. More specifically, the vicinity may be a circular disk with a predefined radius around the first cancer cell. In one embodiment, the continuous spatial proximity score for the first cancer cell is determined by a distance-weighted sum of the several powers of DAB optical densities of membrane and cytoplasm objects whose associated cancer cell centers are closer to the first cancer cell center than a predefined distance. In one embodiment, the predefined distance is 50 μm, as used in the calculation of FIG. 18. In another embodiment, the distance is 20 μm. In another embodiment the distance is determined by minimizing the Kaplan Meier log-rank p-value of spatial-proximity-score-positive arm compared to spatial-proximity-score-negative arm in a predefined training cohort of patients treated with the ADC. In yet another embodiment, the distance weighting involves computing the exponential of the scaled negative Euclidean distance from the first cancer cell center to the other cancer cell centers in the sum. In another embodiment, the powers in the sum are restricted to 0, 1, and 2 (constants, linear terms, squares).

### C. Validation of Binary Spatial Proximity Score

The accuracy of the predictive ADC score generated according to the novel method of FIG. 9 and variations thereof was validated based on patient-derived xenograft (PDX) studies of patients with varying FRα expression levels and correlated to tumor growth as an indication of response to the ADC. Tumor tissue fragments were implanted subcutaneously into female athymic nude mice between 6 to 8 weeks of age. When tumors reached the appropriate tumor volume range (typically 150-300 mm), animals were randomized into treatment and control groups, and dosing with the anti-FRα ADC AB1370049-SG3932-DAR8 was initiated. Dosages of 2.5 mg/kg and 5 mg/kg were used. The human equivalent doses (HED) are 0.2 mg/kg and 0.4 mg·kg, respectively. Tumor-bearing mice were administered a single dose of anti-FRα ADC via intravenous injection. The mice were observed daily, and tumor dimensions and body weight were measured and recorded twice weekly. Tumor volumes were measured by digital caliper and the volumes of tumors were calculated using the following formula: tumor volume=[length (mm) x width (mm)×0.52, where the length and width are the longest and shortest diameters of the tumor, respectively.

In step 15 of method 10, a predicted efficacy score in the form of the binary spatial proximity score (bystander_memb(meanOD)_binary_r25_cut12) is generated for the tissue sample based on the percentage of cancer cells in the digital image that are either optical-density (OD) positive or optical-density negative but within a predefined distance of an optical-density positive cancer cell. In this embodiment, the predefined distance is 25 microns. Each cancer cell is identified as optical-density positive if the mean optical density is greater than or equal to an optical density threshold, and optical-density negative if the mean optical density is less than the optical density threshold. In this embodiment, the optical density threshold is twelve on a scale with a maximum optical density of 220.

In step 15 of method 10, a cancer patient is identified as one who will likely benefit from the administration of the ADC if the binary spatial proximity score exceeds a predetermined threshold. In this embodiment, the predetermined threshold is ninety-eight percent. The predefined distance, the optical density threshold and the predetermined threshold are correlated to responses of training patients treated with the ADC.

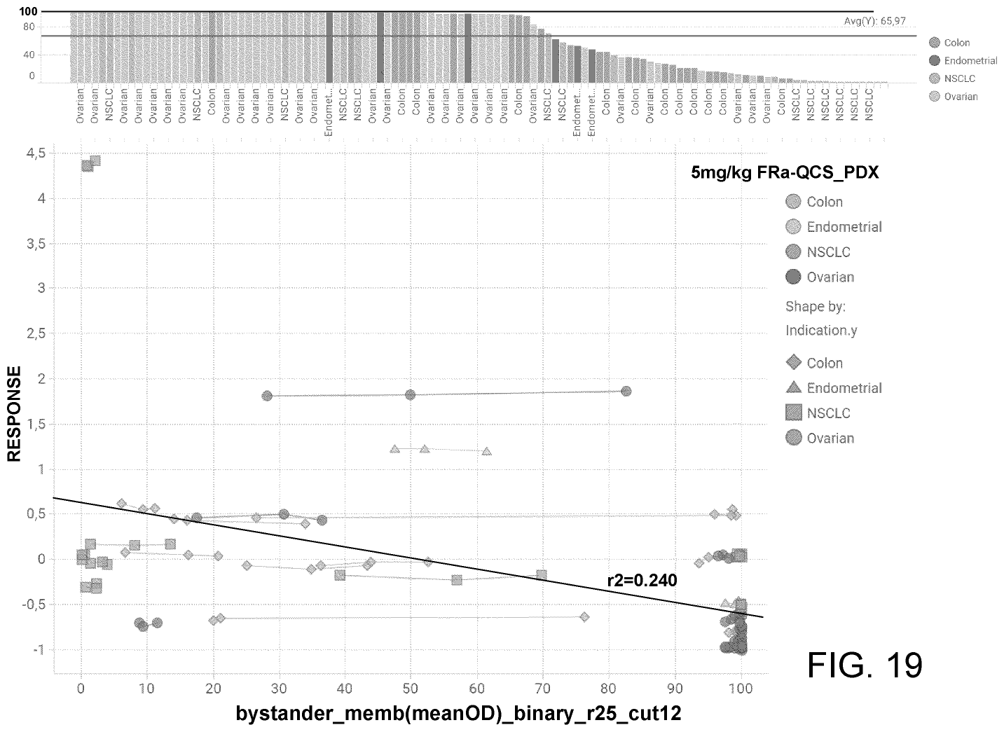

FIG. 19 illustrates how the predefined distance, the optical density threshold and the predetermined threshold were correlated to responses of tissue samples of cancer patients treated with the anti-FRα ADC. FIG. 19 shows the correlation between actual outcomes of PDX studies of 112 patients with ovarian, NSCLC, colon and endometrial cancer. The tissue analyzed in FIG. 19 was that of mice bearing tumor cells from the 112 human cancer patients. A dosage of 5 mg/kg was administered in the PDX studies. FIG. 19 shows the relationship between tumor growth rate (e.g., 1.5=150%) and the predicted efficacy score: bystander_memb(meanOD)_binary_r25 cut12 (percentage of cancer cells that are either (i) optical density (OD) positive or (ii) optical density (OD) negative and within 25 microns of an OD positive cell). Y-axis values greater than 0 denote that the tumor increased in size during observation, and values less than 0 signify that the tumor size shrank during observation.

FIG. 19 shows that there was a negative correlation between the predicted efficacy score bystander_memb(meanOD)_binary_r25_cut12 and the change in tumor size post treatment. In the lower plot of FIG. 19, Spearman's rho is −0.57, and p equals 5.77E-8. Thus, there was a larger decrease in tumor size associated with a higher predicted efficacy score. The upper bar graph of FIG. 19 shows the predicted efficacy score by decreasing magnitude for each of the 112 PDX models of patients with ovarian, NSCLC, colon and endometrial cancer.

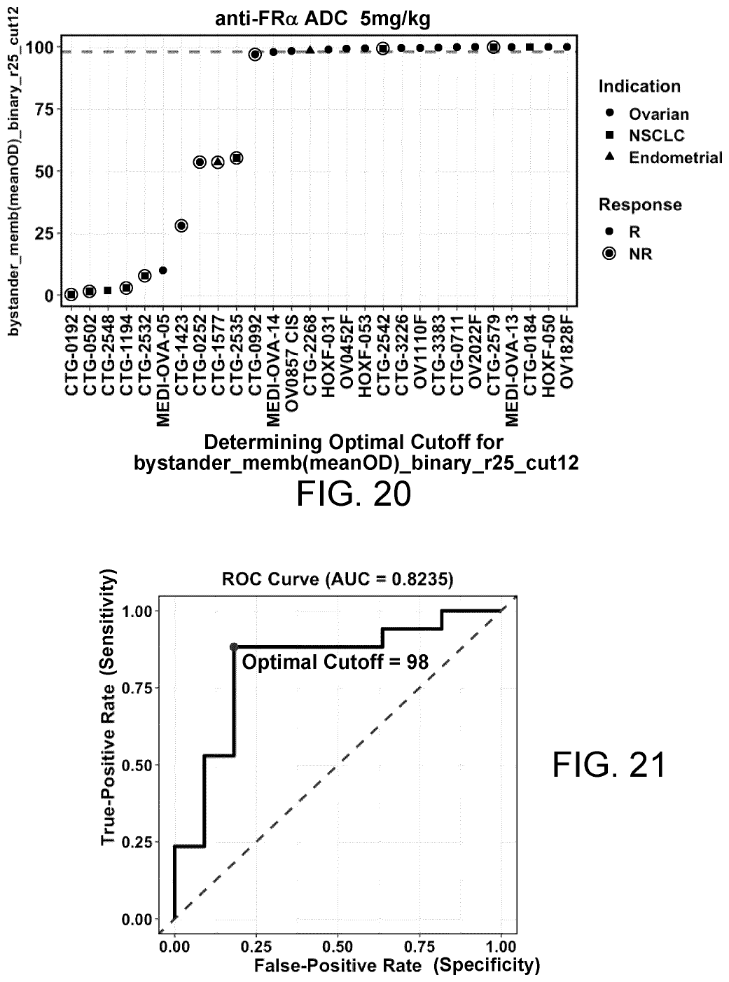

FIG. 20 is a plot showing the predicted efficacy score by increasing magnitude for 28 PDX models of patients with ovarian, NSCLC and endometrial cancer. The predetermined threshold of 98% used in step 16 of method 10 was determined based on the 28 PDX models. Circles represent PDX models with ovarian cancer, squares represent PDX models with lung cancer (NSCLC), and triangles represent PDX models with endometrial cancer. Encircled shapes represent PDX models that were non-responsive to the ADC, and shapes without circles represent PDX models that showed a positive response to the ADC.

FIG. 21 illustrates how the patients were stratified in order to determine the optimal predetermined threshold between responsive and non-responsive patients. FIG. 21 is a receive operating characteristic (ROC) curve used to evaluate the discriminative performance of a predictor, in this case the predicted efficacy score. The “R package cutpointr” ROC curve analysis tool was used to determine the optimal predetermined threshold, as well as prediction performance parameters such as the AUC (area under curve), the Youden index value, sensitivity and specificity. Values of the AUC closer to 1 indicate better discriminative performance at patient stratification between binary clinical outcomes, such as responsive (R) and non-responsive (NR). The sensitivity is the true-positive rate and equals the number of true positives divided by the sum of true positives and false negatives [TP/(TP+FN)]. The sensitivity indicates the probability that a positive score indeed corresponds to a positive clinical outcome. The specificity is the false-positive rate and equals the number of true negatives divided by the sum of true negatives and false positives [TN/(TN+FP)]. The specificity indicates the probability that a positive score actually corresponds to a negative clinical outcome. The Youden index value equals the sensitivity plus the specificity minus 1.

For the predicted efficacy score bystander_memb(meanOD)_binary_r25_cut12, FIG. 21 illustrates that the optimal predetermined threshold is 98%, and the area under curve (AUC) is 0.8235. The sensitivity is 0.8824, the specificity is 0.8182, and the Youden index value is 0.7005.

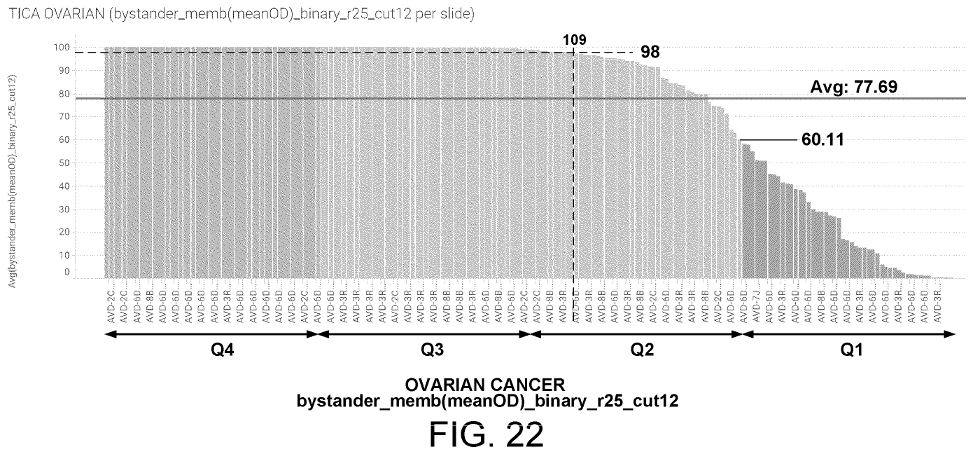

FIG. 22 shows the distribution of values of the predicted efficacy score bystander_memb(meanOD)_binary_r25 cut12 in 197 tissue samples from a database of ovarian cancer patients. Stained tissue samples of the human patients were analyzed as opposed to tissue from PDX models. The bar graph of decreasing score values indicates the prevalence among ovarian cancer patients of various ranges of the predicted efficacy score (binary spatial proximity score). One quarter of the patients had a score of 60.11% or less. The average score was 77.69%. FIG. 22 indicates that the tissue samples from more than half (109) of the 197 ovarian cancer patients resulted in a bystander_memb(meanOD)_binary_r25_cut12 score of 98% or greater. These ovarian cancer patients would likely have a positive response to a therapy involving an anti-FRα antibody-drug conjugate.

In step 17 of method 10 of FIG. 9, a therapy involving the anti-FRα ADC is recommended to the cancer patient if the predicted efficacy score exceeds the predetermined threshold of 98%. Thus, the anti-FRα ADC therapy would be recommended to 109 of the 197 ovarian cancer patients.

### D. Validation of Percent Cells Having Minimum OD

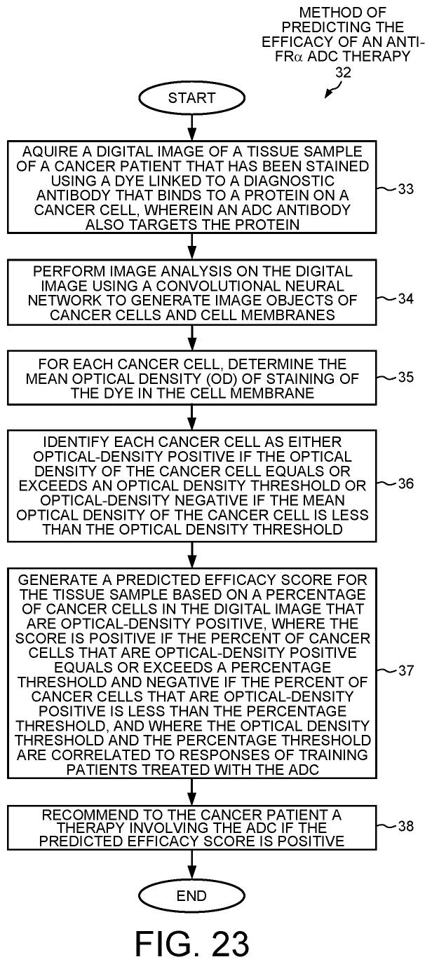

Variations of the novel method of FIG. 9 were also validated by treating tumors in the PDX models with the anti-FRα ADC AB1370049-SG3932-DAR8 in dosages of 2.5 mg/kg and 5 mg/kg. FIG. 23 is a flowchart of steps 33-38 of a method 32 of predicting the response of a cancer patient to the anti-FRα ADC. Method 32 is another embodiment of method 10 of FIG. 9 and predicts the efficacy of a therapy involving an anti-FRα ADC based on the percentage of cancer cells that exhibit a minimum amount of staining. In step 33, a digital image is acquired of a tissue slice from the cancer patient that has been stained using a dye linked to a diagnostic antibody that targets the same protein as that targeted by the ADC therapy, in this case the FRα protein. In step 34, image analysis is performed on the digital image to generate image objects of cancer cells and cell membranes. In step 35, the mean optical density (OD) of staining of the dye in the cell membrane is determined for each cancer cell.

In step 36, each cancer cell is identified as being either optical-density positive or optical-density negative based on the mean optical density of staining of the cell membrane. A cancer cell is identified as optical-density positive if the mean optical density is greater than or equal to an optical density threshold. A cancer cell is optical-density negative if the mean optical density is less than the optical density threshold. In this embodiment, the optical density threshold is twelve on a scale with a maximum optical density of 220.

In step 37, a predicted efficacy score is generated for the tissue sample based on the percentage of cancer cells in the digital image that are optical-density positive. The predicted efficacy score is positive if the percentage of cancer cells that are optical-density positive is greater than or equal to a percentage threshold. The predicted efficacy score is negative if the percentage of cancer cells that are optical-density positive is less than the percentage threshold. The optical density threshold and the percentage threshold are correlated to responses of training patients treated with the ADC. In this embodiment, the percentage threshold is ninety percent.

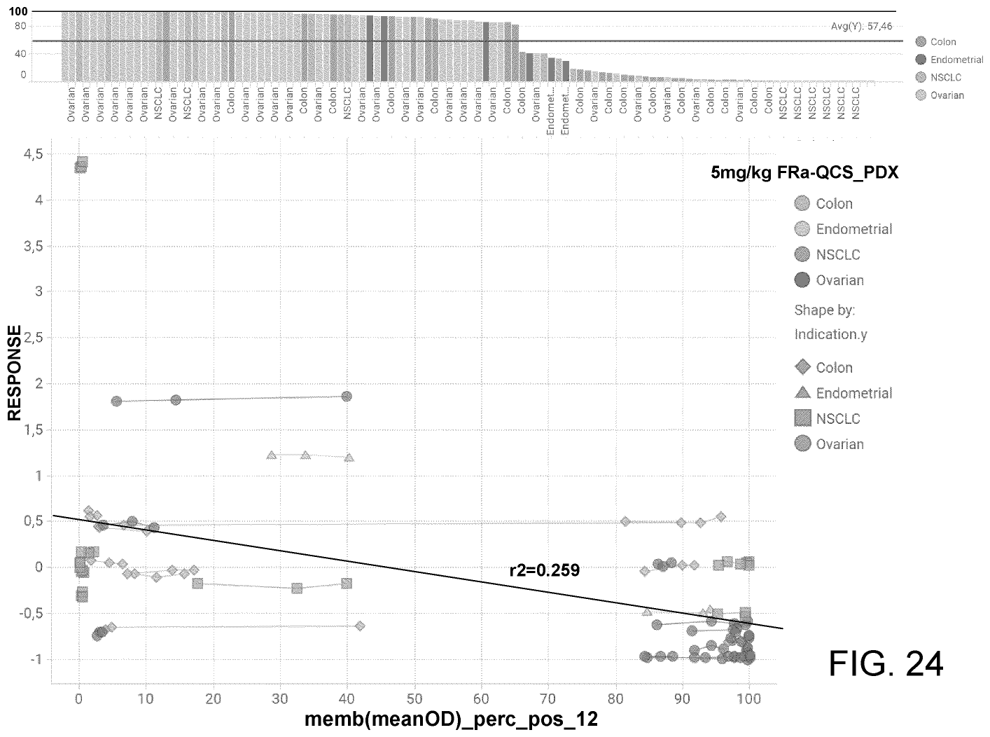

FIG. 24 illustrates how the optical density threshold and the percentage threshold were correlated to responses of tissue samples of cancer patients treated with the anti-FRα ADC. FIG. 24 shows the correlation between the actual outcomes of PDX studies of 112 patients with ovarian, NSCLC, colon and endometrial cancer. The tissue analyzed in FIG. 24 was that of mice bearing tumor cells from the 112 human cancer patients. A dosage of 5 mg/kg was administered in the PDX studies. FIG. 24 shows the relationship between tumor growth in percent (1=100%) and the predicted efficacy score memb(meanOD)_perc_pos_12 (percentage of cancer cells whose mean optical density of staining is at least 12 on a scale of 220). Y-axis values greater than 0 denote that the tumor increased in size during observation, and values less than 0 means that the tumor size shrank during observation.

FIG. 24 shows that there was a negative correlation between the predicted efficacy score memb(meanOD)_perc_pos_12 and the change in tumor size post treatment. In the lower plot of FIG. 24, Spearman's rho is −0.54, and p equals 3.36E-7. Thus, there was a larger decrease in tumor size associated with a higher predicted efficacy score. The upper bar graph of FIG. 24 shows the predicted efficacy score ordered by decreasing magnitude for each of the 112 PDX models of patients with ovarian, NSCLC, colon and endometrial cancer.

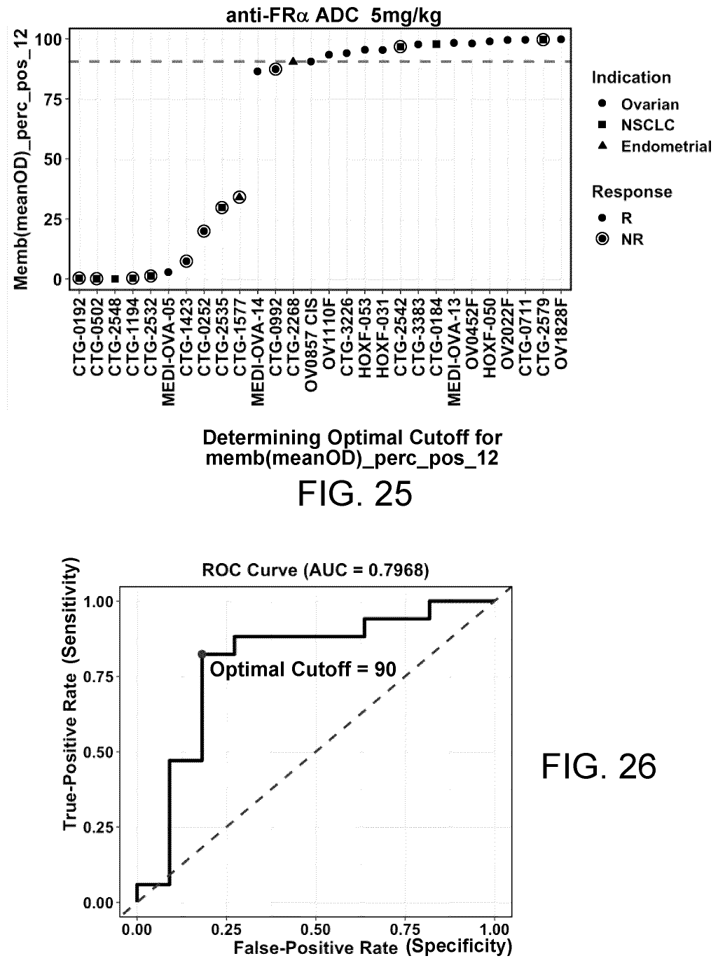

FIG. 25 is a plot showing the predicted efficacy score ordered by increasing magnitude for 28 PDX models of patients with ovarian, NSCLC and endometrial cancer. The percentage threshold of 90% used in step 37 of method 32 was determined based on the 28 PDX models. Circles represent PDX models with ovarian cancer, squares represent PDX models with lung cancer (NSCLC), and triangles represent PDX models with endometrial cancer. Encircled shapes represent PDX models that were non-responsive to the ADC, and shapes without circles represent PDX models that showed a positive response to the ADC.

FIG. 26 illustrates how the patients were stratified in order to determine the optimal percentage threshold between responsive and non-responsive patients. FIG. 26 is a receive operating characteristic (ROC) curve used to evaluate the discriminative performance of a predictor, in this case the memb(meanOD)_perc_pos_12 score. The same ROC curve analysis tool used with FIG. 20 was used to determine the optimal percentage threshold in FIG. 25. For the predicted efficacy score memb(meanOD)_perc_pos_12, FIG. 26 illustrates that the optimal percentage threshold is 90%, and the area under curve (AUC) is 0.7968. The sensitivity is 0.8235, the specificity is 0.8182, and the Youden index value is 0.6417.

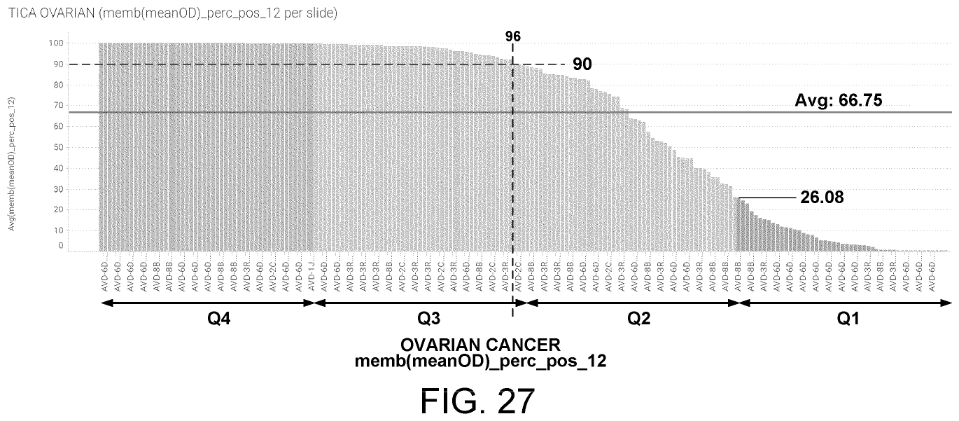

FIG. 27 shows the distribution of values of the predicted efficacy score memb(meanOD)_perc_pos_12 in 197 tissue samples from a database of ovarian cancer patients. Stained tissue samples of the human patients were analyzed in FIG. 27 as opposed to tissue from PDX models. The bar graph of decreasing score values indicates the prevalence among ovarian cancer patients of various ranges of the predicted efficacy score. One quarter of the patients had a score of 26.08% or less. The average score was 66.75%. FIG. 27 indicates that the tissue samples from just under half (96) of the 197 ovarian cancer patients resulted in a memb(meanOD)_perc_pos_12 score of 90% or greater. These ovarian cancer patients would likely have a positive response to a therapy involving an anti-FRα antibody-drug conjugate.

In step 38 of method 32 of FIG. 23, a therapy involving the anti-FRα ADC is recommended to the cancer patient if the predicted efficacy score is positive. The score is positive if the memb(meanOD)_perc_pos_12 score is 90% or greater. Thus, the anti-FRα ADC therapy would be recommended to just under half of the 197 ovarian cancer patients.

### E. Validation of Median Absolute Deviation of OD

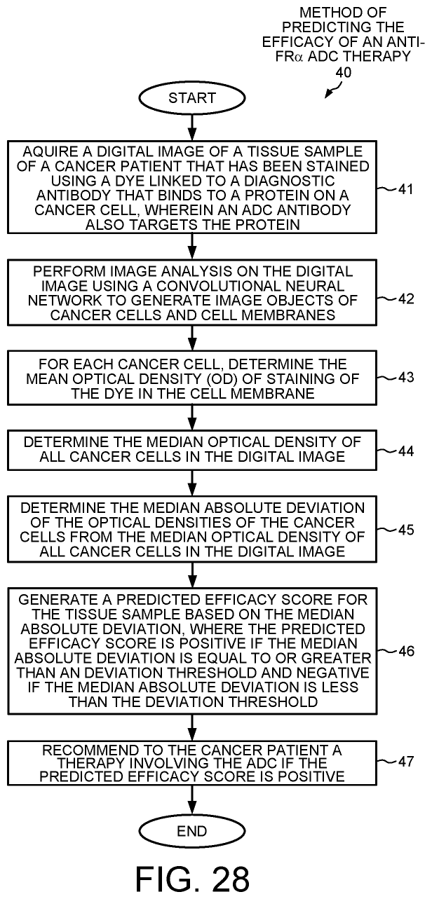

Another embodiment of method 10 of FIG. 9 involves determining the median of the absolute deviation of staining of each cell from the median membrane staining of all cancer cells in the digital image. FIG. 28 is a flowchart of steps 41-47 of a method 40 for predicting the response of a cancer patient to the anti-FRα ADC. Method 40 predicts the efficacy of a therapy involving an anti-FRα ADC based on the absolute deviation of membrane staining in the digital image. In step 41, a digital image is acquired of a tissue slice from the cancer patient that has been stained using a dye linked to a diagnostic antibody that targets the FRα protein. In step 42, image analysis is performed on the digital image to generate image objects of cancer cells and cell membranes. In step 43, the mean optical density (OD) of staining of the dye in the cell membrane is determined for each cancer cell. In step 44, the median optical density of all cancer cells in the digital image is determined.

In step 45, the median absolute deviation is determined of the optical densities of the cancer cells from the median optical density of all cancer cells in the digital image. In step 46, a predicted efficacy score is generated for the tissue sample based on the median absolute deviation. The predicted efficacy score is positive if the median absolute deviation is equal to or greater than a deviation threshold and negative if the median absolute deviation is less than the deviation threshold. The deviation threshold is correlated to responses of a cohort of training patients treated with the ADC. In this embodiment, the deviation threshold is twenty-four percent.

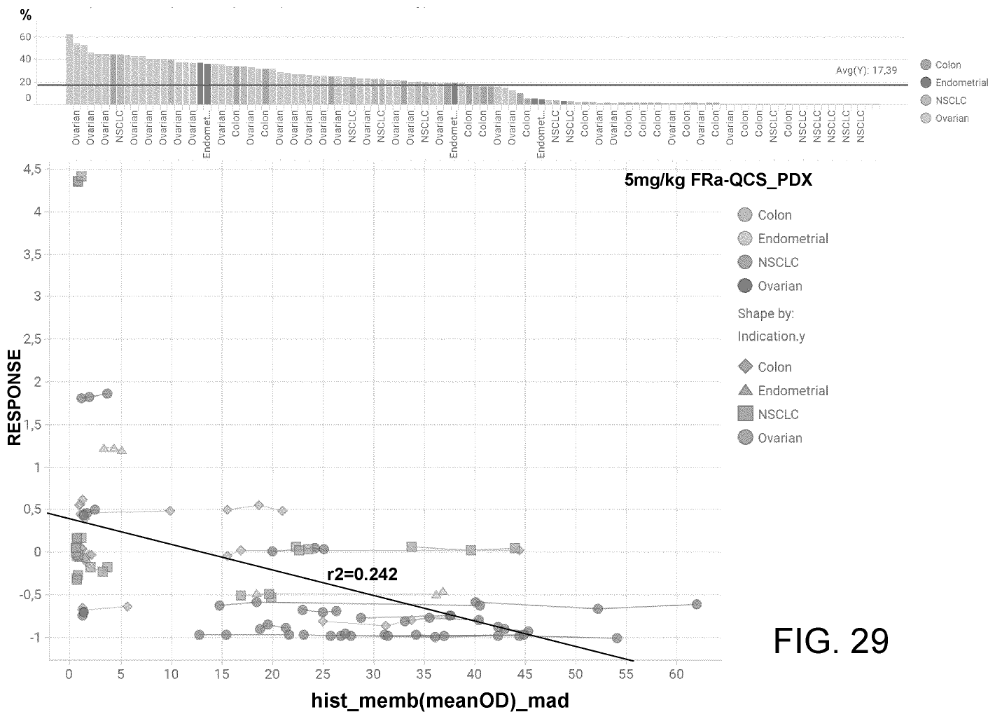

FIG. 29 illustrates how the optical density threshold and the deviation threshold were correlated to responses of tissue samples of cancer patients treated with the anti-FRα ADC. FIG. 29 shows the correlation between the actual outcomes of PDX studies of 112 patients with ovarian, NSCLC, colon and endometrial cancer. The tissue analyzed in FIG. 29 was that of mice bearing tumor cells from the 112 human cancer patients. A dosage of 5 mg/kg was administered in the PDX studies. FIG. 29 shows the relationship between tumor growth in percent (1=100%) and the predicted efficacy score hist_memb(meanOD)_mad (median absolute deviation of optical densities of cancer cells from median optical density of all cancer cells in digital image). Y-axis values greater than 0 denote that the tumor increased in size during observation, and values less than 0 means that the tumor size shrank during observation.

FIG. 29 shows that there was a negative correlation between the predicted efficacy score hist_memb(meanOD)_mad and the change in tumor size post treatment. In the lower plot of FIG. 29, Spearman's rho is −0.60, and p equals 7.03E-9. Thus, there was a larger decrease in tumor size associated with a higher predicted efficacy score. The upper bar graph of FIG. 29 shows the predicted efficacy score ordered by decreasing magnitude for each of the 112 PDX models of patients with ovarian, NSCLC, colon and endometrial cancer.

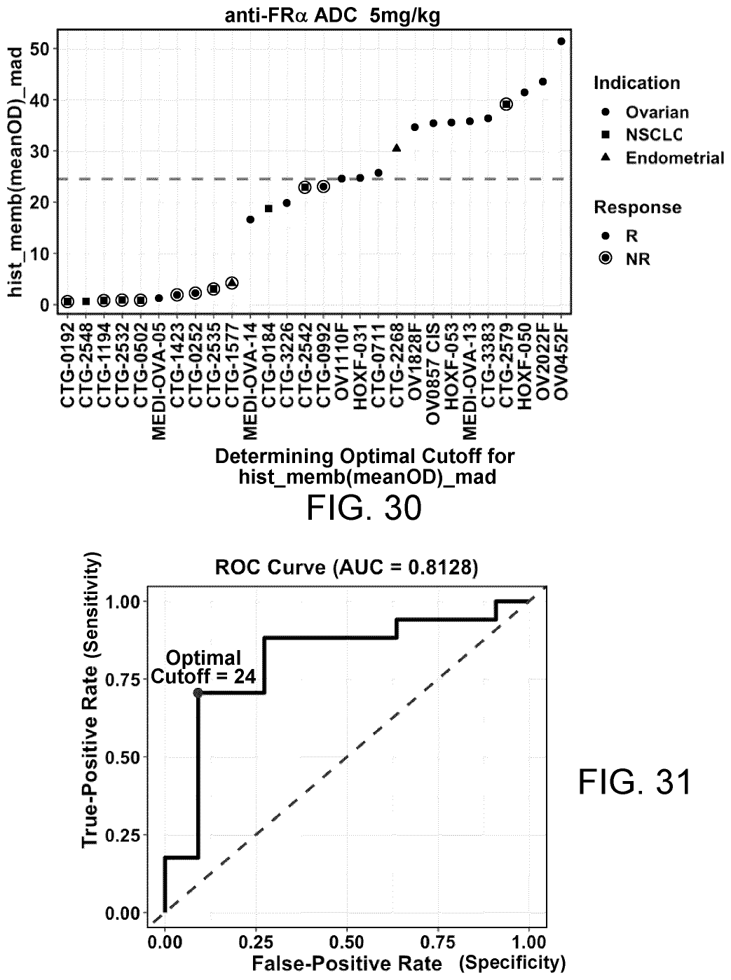

FIG. 30 is a plot showing the predicted efficacy score ordered by increasing magnitude for 28 PDX models of patients with ovarian, NSCLC and endometrial cancer. The deviation threshold of 24% used in step 46 of method 40 was determined based on the 28 PDX models. Circles represent PDX models with ovarian cancer, squares represent PDX models with lung cancer (NSCLC), and triangles represent PDX models with endometrial cancer. Encircled shapes represent PDX models that were non-responsive to the ADC, and shapes without circles represent PDX models that showed a positive response to the ADC.

FIG. 31 illustrates how the patients were stratified in order to determine the optimal deviation threshold between responsive and non-responsive patients. FIG. 31 is a receive operating characteristic (ROC) curve used to evaluate the discriminative performance of a predictor, in this case the hist_memb(meanOD)_mad score. The same ROC curve analysis tool used with FIG. 20 was used to determine the optimal deviation threshold in FIG. 30. For the predicted efficacy score hist_memb(meanOD)_mad, FIG. 31 illustrates that the optimal deviation threshold is 24%, and the area under curve (AUC) is 0.8128. The sensitivity is 0.7059, the specificity is 0.9091, and the Youden index value is 0.6150.

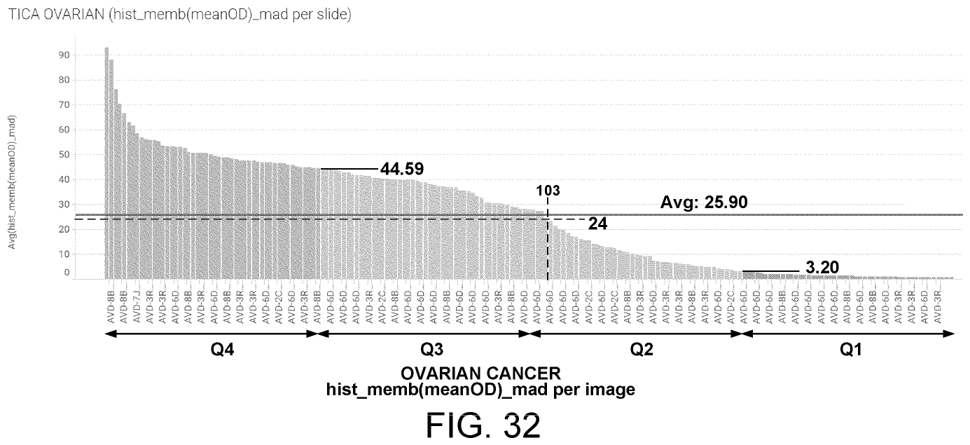

FIG. 32 shows the distribution of values of the predicted efficacy score hist_memb(meanOD) mad in 197 tissue samples from a database of ovarian cancer patients. Stained tissue samples of the human patients were analyzed in FIG. 32 as opposed to tissue from the PDX models shown in FIG. 30. The bar graph of decreasing score values indicates the prevalence among ovarian cancer patients of various ranges of the predicted efficacy score. One quarter of the patients had a score of 3.20% or less. One quarter of the patients had a score of 44.59% or more. The average score was 25.90%. FIG. 32 indicates that the tissue samples from just over half (103) of the 197 ovarian cancer patients resulted in a hist_memb(meanOD)_mad score of 24% or greater. These ovarian cancer patients would likely have a positive response to a therapy involving an anti-FRα antibody-drug conjugate.

In step 47 of method 40 of FIG. 28, a therapy involving the anti-FRα ADC is recommended to the cancer patient if the predicted efficacy score is positive. The score is positive if the hist_memb(meanOD)_mad score is 24% or greater. Thus, the anti-FRα ADC therapy would be recommended to just over half of the 197 ovarian cancer patients.

### F. Validation of Mean OD to Predict Dosage Efficacy

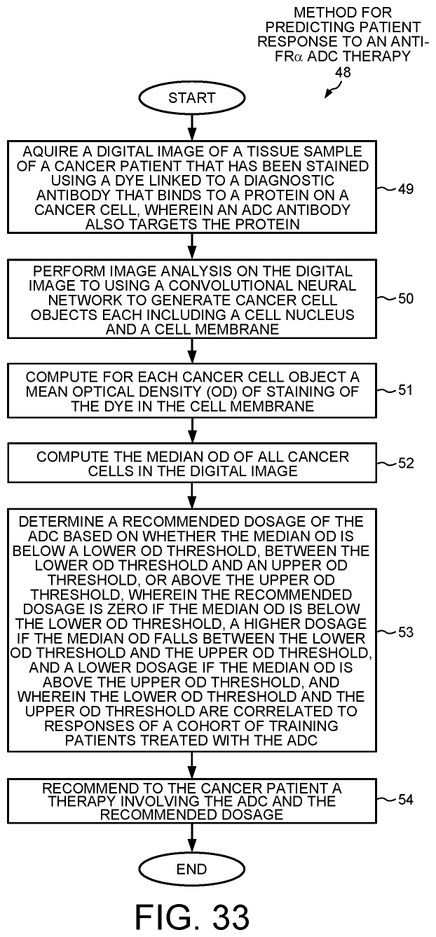

Another embodiment of method 10 of FIG. 9 involves determining the median optical density of all cancer cells in the digital image in order to determine the recommended dosage of the anti-FRα ADC. FIG. 33 is a flowchart of steps 49-54 of a method 48 for predicting the efficacy of various dosages of the anti-FRα ADC based on the median optical density of membrane staining in the digital image. In step 49, a digital image is acquired of a tissue slice from the cancer patient that has been stained using a dye linked to a diagnostic antibody that targets the FRα protein. In step 50, image analysis is performed on the digital image to generate image objects of cancer cells and cell membranes. In step 51, the mean optical density (OD) of staining of the dye in the cell membrane is determined for each cancer cell. In step 52, the median optical density of all cancer cells in the digital image is determined. This is the equivalent of determining the optical density of the fifty percent quantile (q50) of cancer cells in the digital image, which is a whole slide image (WSI).

In step 53, the recommended dosage of the anti-FRα ADC is determined based on whether the median optical density falls (i) below a lower optical density threshold, (ii) between the lower optical density threshold and an upper optical density threshold, or (iii) above the upper optical density threshold. The recommended dosage is zero if the median optical density falls below the lower optical density threshold. Thus, the ADC is not administered to the cancer patient if the median optical density is below the lower optical density threshold. The recommended dosage is a higher dosage if the median optical density falls between the lower optical density threshold and the upper optical density threshold. The recommended dosage is a lower dosage if the median optical density falls above the upper optical density threshold. The lower optical density threshold and the upper optical density threshold are correlated to responses of a cohort of training patients treated with the ADC. In this embodiment, the lower dosage is 2.5 mg/kg, and the higher dosage is 5 mg/kg. In this embodiment, the lower optical density threshold is 25, and the upper optical density threshold is 39, both on a scale with a maximum optical density of 220.

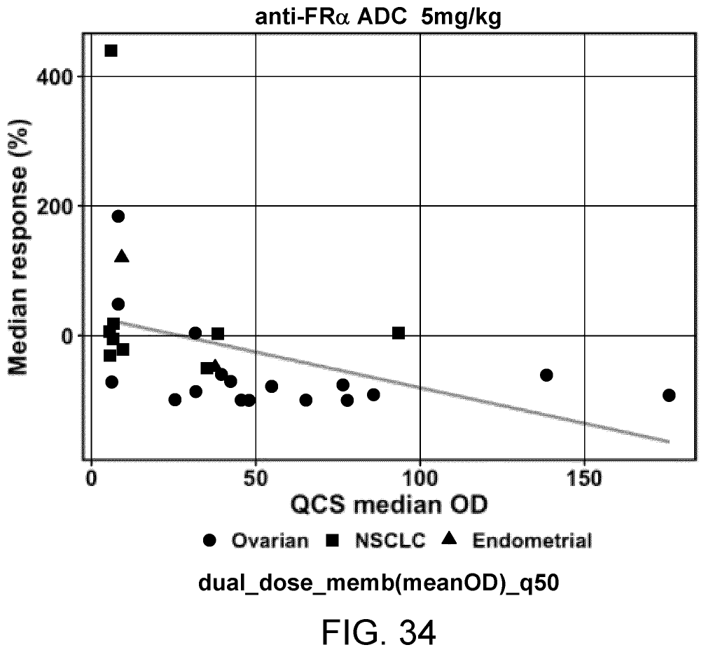

FIGS. 34-39 illustrate how the lower optical density threshold and the upper optical density threshold were correlated to responses of tissue samples of cancer patients treated with the anti-FRα ADC. FIG. 34 shows the correlation between the actual outcomes of PDX studies of 28 patients with ovarian, NSCLC and endometrial cancer. The tissue analyzed in FIG. 34 was that of mice bearing tumor cells from the 28 human cancer patients. A dosage of 5 mg/kg was administered in the PDX studies. FIG. 34 shows the relationship between tumor growth in percent and the predicted efficacy score dual_dose_memb(meanOD)_q50 (mean OD of membrane staining). Y-axis values greater than 0 denote that the tumor increased in size during observation, while values less than 0 signify that the tumor size shrank during observation.

FIG. 34 shows that there was a negative correlation between the predicted efficacy score dual_dose_memb(meanOD)_q50 and the change in tumor size post treatment. There was a larger decrease in tumor size associated with a higher predicted efficacy score.

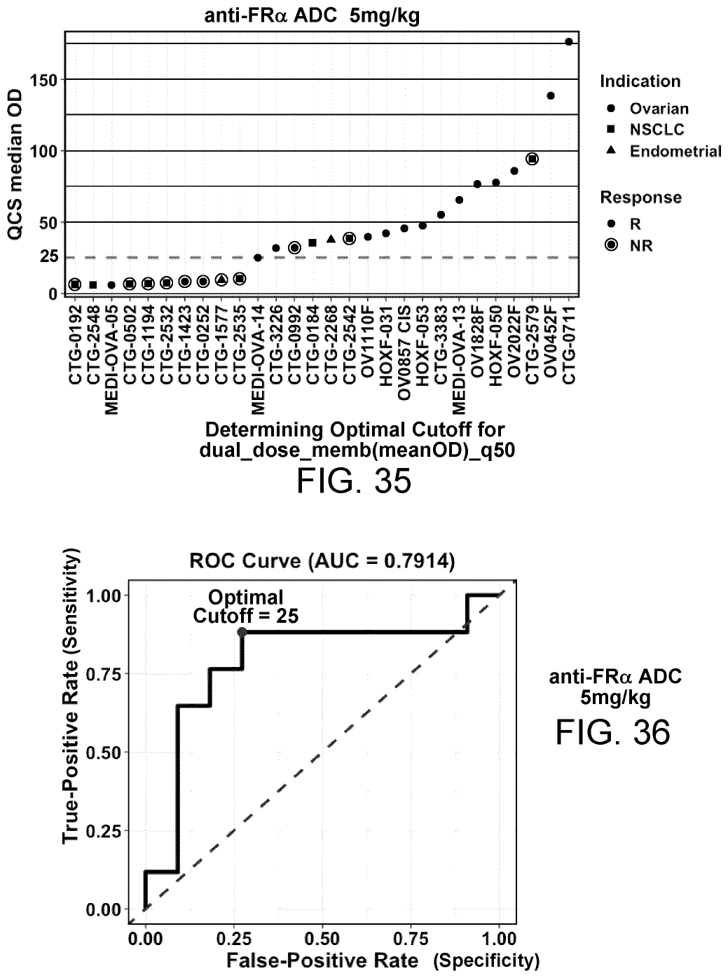

FIG. 35 is a plot showing the predicted efficacy score ordered by increasing magnitude for the 28 PDX models of patients with ovarian, NSCLC and endometrial cancer shown in FIG. 34. The lower optical density threshold of 25 used in step 53 of method 48 was determined based on the 28 PDX models that were treated with 5 mg/kg of the anti-FRα ADC. Circles represent PDX models with ovarian cancer, squares represent PDX models with lung cancer (NSCLC), and triangles represent PDX models with endometrial cancer. Encircled shapes represent PDX models that were non-responsive to the ADC, and shapes without circles represent PDX models that showed a positive response to the ADC.

FIG. 36 illustrates how the patients were stratified in order to determine the optimal lower optical density threshold between responsive and non-responsive patients. FIG. 36 is a receive operating characteristic (ROC) curve used to evaluate the discriminative performance of a predictor, in this case the dual_dose_memb(meanOD)_q50 score. For the predicted efficacy score for PDX models treated with 5 mg/kg of the anti-FRα ADC, FIG. 36 illustrates that the optimal lower optical density threshold is an optical density of 25, and the area under curve (AUC) is 0.7914. The sensitivity is 0.8824, the specificity is 0.7273, and the Youden index value is 0.6096.

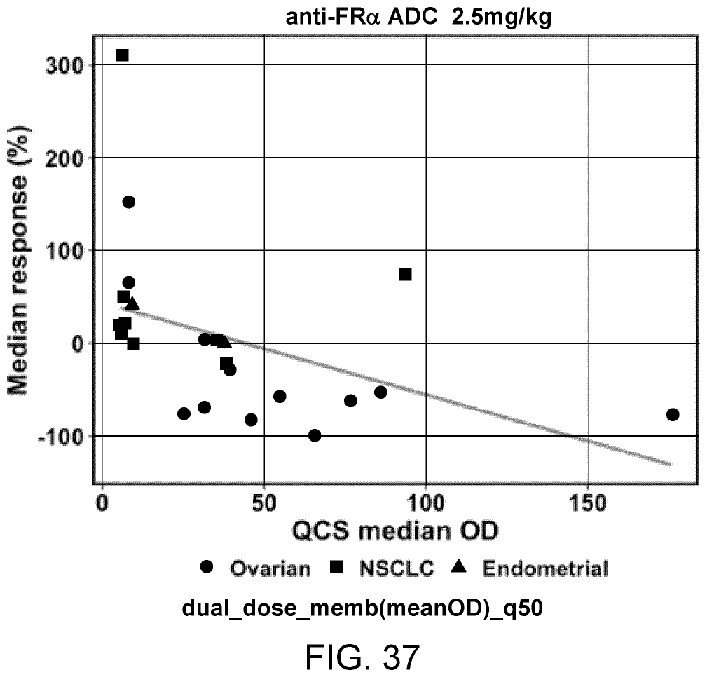

FIG. 37 illustrates how the upper optical density threshold was correlated to responses of tissue samples of cancer patients treated with the anti-FRα ADC. FIG. 37 shows the correlation between the actual outcomes of PDX studies of 23 patients with ovarian, NSCLC and endometrial cancer. A dosage of 2.5 mg/kg was administered in the PDX studies. The tissue analyzed in FIG. 37 was that of mice bearing tumor cells from the 23 human cancer patients. FIG. 37 shows the relationship between the predicted efficacy score dual_dose_memb(meanOD)_q50 and tumor growth in percent after administering a dosage of 2.5 mg/kg. FIG. 37 shows that there was a negative correlation between the predicted efficacy score dual_dose_memb(meanOD)_q50 and the change in tumor size post treatment. There was a larger decrease in tumor size associated with a higher predicted efficacy score.

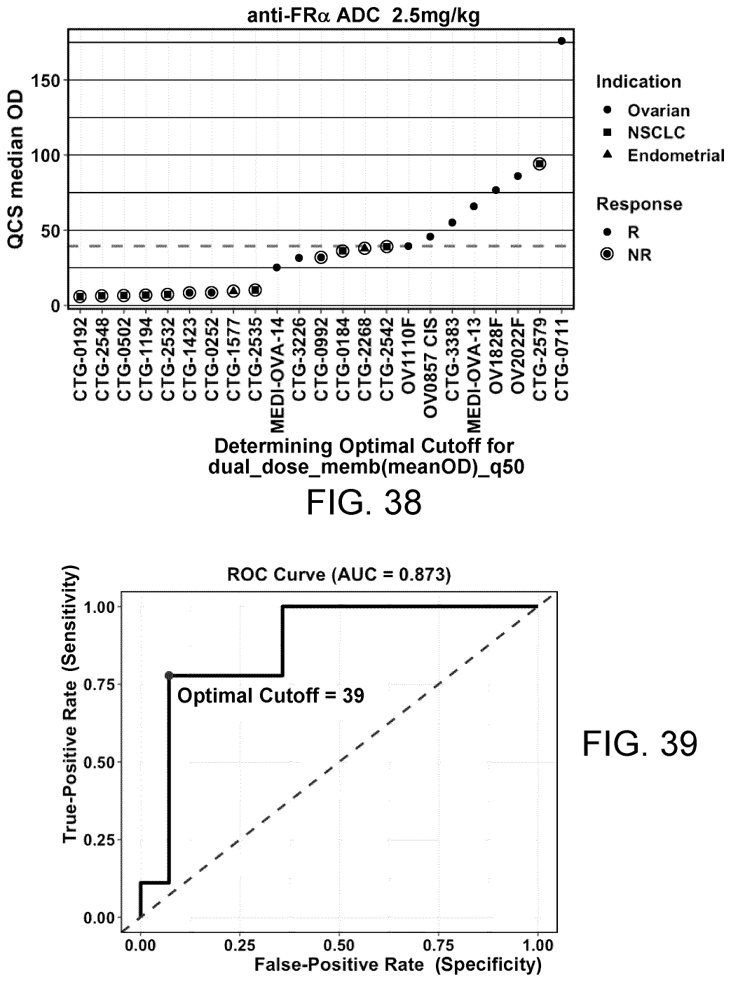

FIG. 38 is a plot showing the predicted efficacy score ordered by increasing magnitude for the 23 PDX models of patients with ovarian, NSCLC and endometrial cancer shown in FIG. 37. The upper optical density threshold of 39 used in step 53 of method 48 was determined based on the 23 PDX models that were treated with 2.5 mg/kg of the anti-FRα ADC.

FIG. 39 illustrates how the patients were stratified in order to determine the optimal upper optical density threshold between responsive and non-responsive patients. FIG. 39 is the ROC curve used to evaluate the discriminative performance of the dual_dose_memb(meanOD)_q50 predicted efficacy score. For the predicted efficacy score for PDX models treated with 2.5 mg/kg of the anti-FRα ADC, FIG. 39 illustrates that the optimal upper optical density threshold is an optical density of 39, and the area under curve (AUC) is 0.8730. The sensitivity is 0.7778, the specificity is 0.9286, and the Youden index value is 0.7063.

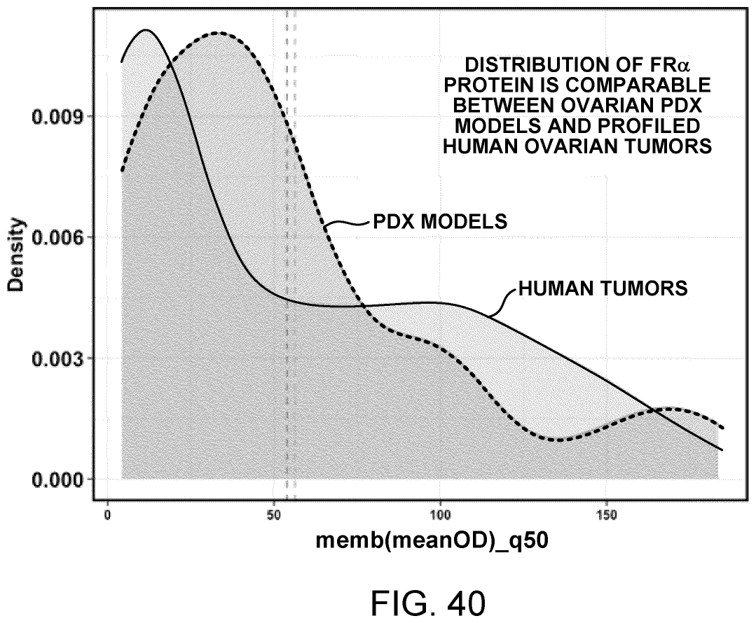

FIG. 40 is a graph of median optical density for each of multiple PDX models compared to the median optical density for human ovarian tumors. One curve shows the median optical density of membrane staining for 197 human ovarian tumors and includes a dashed line marking the mean of all the median optical density scores. The other curve shows the median optical density of membrane staining for 18 PDX models and includes a dashed line at the mean of the median optical density scores that is only slightly to the left of the dashed line for the human ovarian tumors. The graph demonstrates that the distribution of FRα protein in ovarian cancer PDX models is comparable to that in profiled human ovarian tumors, and there is a similar FRα distribution in patient-derived xenografts and in human tumors of ovarian cancer. Thus, the PDX models can be used to determine the thresholds for the various predicted efficacy scores.

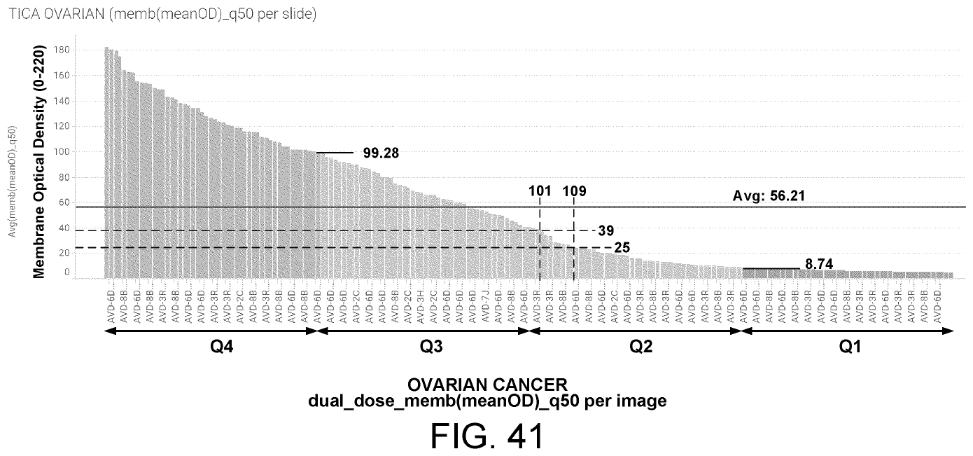

FIG. 41 shows the distribution of values of the predicted efficacy score dual_dose_memb(meanOD)_q50 in 197 tissue samples from a database of ovarian cancer patients. Stained tissue samples of the human patients were analyzed in FIG. 41 as opposed to tissue from the PDX models shown in FIGS. 34-39. The bar graph of decreasing score values indicates the prevalence among ovarian cancer patients of various ranges of the predicted efficacy score. One quarter of the patients had a score of optical density equaling 8.74 or less, and another quarter of the patients had a score of optical density equaling 99.28 or greater. The average score was an optical density of 56.21. FIG. 41 illustrates that the tissue samples from 101 of the 197 ovarian cancer patients resulted in a dual_dose_memb(meanOD)_q50 score of 39 or greater. These ovarian cancer patients would likely have a positive response to the lower dosage of 2.5 mg/kg of the anti-FRα antibody-drug conjugate. An additional 8 ovarian cancer patients would likely have a positive response to the higher dosage of 5 mg/kg of the anti-FRα antibody-drug conjugate. These additional 8 patients would likely no achieve a positive response with only the lower dosage of 2.5 mg/kg. While the 101 patients would likely also have a positive response with the higher 5 mg/kg dosage of the ADC, these patients can avoid any harmful side effects of the additional ADC dosage, which is not required in order for these patients to achieve a positive response. The recommended dosage is zero for the 88 patients whose predicted efficacy score is less than 25. A therapy involving the anti-FRα ADC would not be recommended to the 88 patients.

In step 54 of method 48 of FIG. 33, a therapy involving the anti-FRα ADC at the recommended dosage is recommended to the cancer patient based on whether the predicted efficacy score for the digital image of the patient's tissue sample is below the lower optical density threshold of 25, between the lower optical density threshold of 25 and the upper optical density threshold of 39, or above the upper optical density threshold of 39. The higher dosage of 5 mg/kg is recommended if the predicted efficacy score is between 25 and 39, and the lower dosage of 2.5 mg/kg is recommended if the predicted efficacy score is above 39. The recommended dosage is zero if the predicted efficacy score is below 25.

### G. Validation of Difference Between Membrane OD and Cytoplasm OD

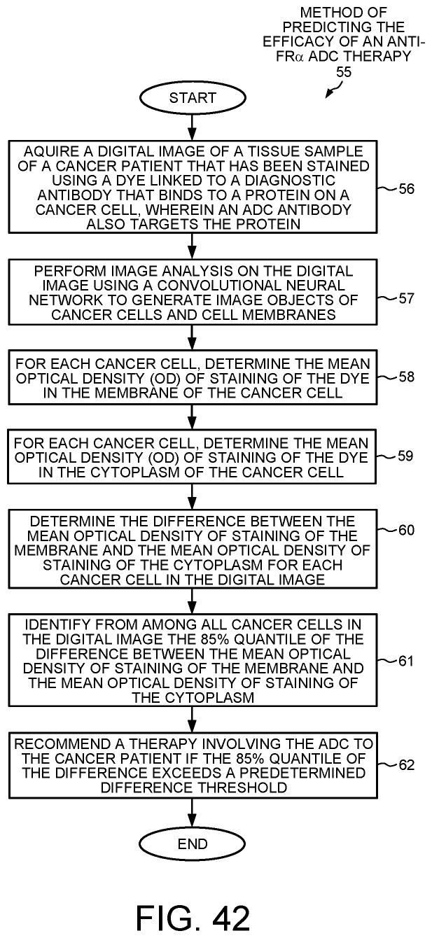

Yet another embodiment of the novel method for generating a predicted efficacy score involves determining the difference between the optical density of membrane staining and the optical density of cytoplasm staining. FIG. 42 is a flowchart of steps 56-62 of a method 55 for predicting the response of a cancer patient to the anti-FRα ADC. Method 55 predicts the efficacy of a therapy involving an anti-FRα ADC by identifying the 85% quantile of the difference between the optical densities of membrane staining and cytoplasm staining. In step 56, a digital image is acquired of a tissue slice from the cancer patient that has been stained using a dye linked to a diagnostic antibody that targets the FRα protein. In step 57, image analysis is performed on the digital image to generate image objects of cancer cells, cytoplasm and cell membranes. In step 58, the mean optical density (OD) of staining of the dye in the cell membrane is determined for each cancer cell. In step 59, the mean optical density of staining of the dye in the cell cytoplasm is determined for each cancer cell. In step 60, the difference between the mean optical density of staining of the membrane and the mean optical density of staining of the cytoplasm is determined for each cancer cell in the digital image. In step 61, the 85% quantile of the difference between the optical density of membrane staining and the optical density of cytoplasm staining is identified from among all cancer cells in the digital image. In step 62, a therapy involving the anti-FRα ADC is recommended to the cancer patient if the 85% quantile of the optical density difference exceeds a predetermined difference threshold. The difference threshold is correlated to responses of a cohort of training patients treated with the ADC. In this embodiment, the difference threshold is 23.

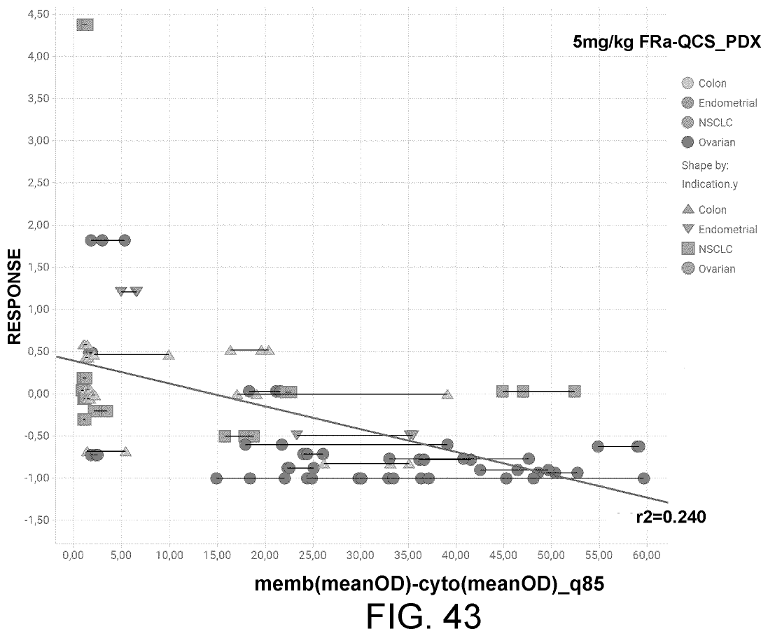

FIG. 43 illustrates how the difference threshold was correlated to responses of tissue samples of cancer patients treated with the anti-FRα ADC. FIG. 43 shows the correlation between the actual outcomes of PDX studies of patients with ovarian, NSCLC, colon and endometrial cancer. The tissue analyzed in FIG. 43 was that of mice bearing tumor cells from the human cancer patients. A dosage of 5 mg/kg was administered in the PDX studies. FIG. 43 shows the relationship between tumor growth in percent (e.g., 2.5=250%) and the predicted efficacy score memb(meanOD)-cyto(meanOD)_q85 (85% quantile of difference between OD of membrane staining and OD of cytoplasm staining). Y-axis values greater than 0 denote that the tumor increased in size during observation, and values less than 0 means that the tumor size shrank during observation. FIG. 43 shows that there was a negative correlation between the predicted efficacy score memb(meanOD)-cyto(meanOD)_q85 and the change in tumor size post treatment. In the plot of FIG. 43, Spearman's rho is −0.62, and p equals 1.50E-9. Thus, there was a larger decrease in tumor size associated with a higher predicted efficacy score.

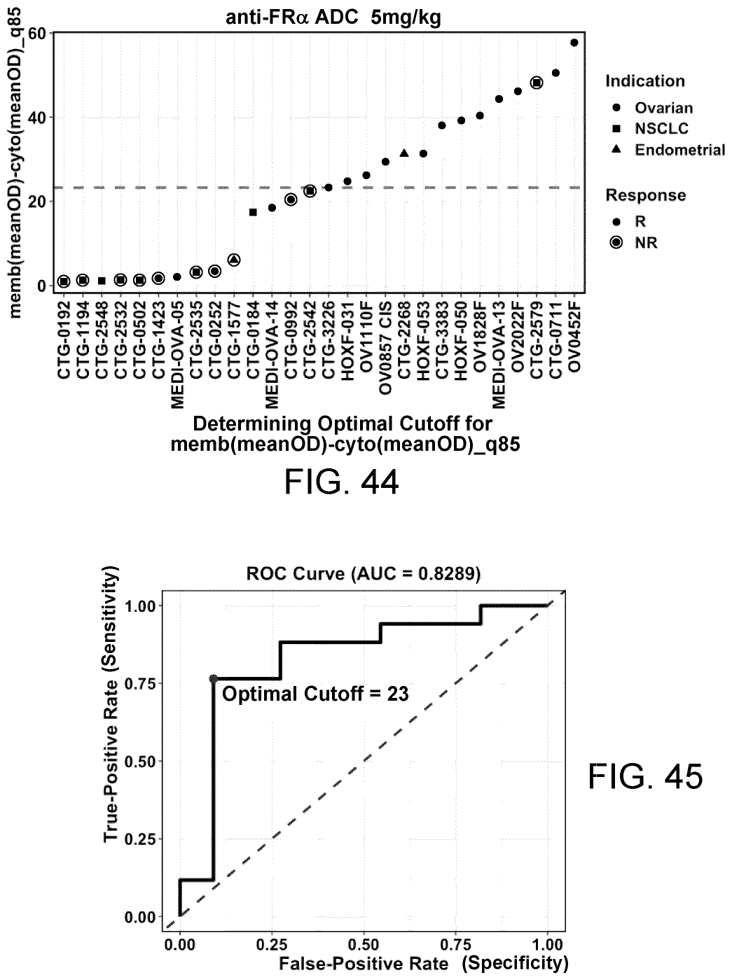

FIG. 44 is a plot showing the predicted efficacy score ordered by increasing magnitude for 28 PDX models of patients with ovarian, NSCLC and endometrial cancer. The difference threshold of 23 used in step 62 of method 55 was determined based on the 28 PDX models. Circles represent PDX models with ovarian cancer, squares represent PDX models with lung cancer (NSCLC), and triangles represent PDX models with endometrial cancer. Encircled shapes represent PDX models that were non-responsive to the ADC, and shapes without circles represent PDX models that showed a positive response to the ADC.

FIG. 45 illustrates how the patients were stratified in order to determine the optimal difference threshold between responsive and non-responsive patients. FIG. 45 is an ROC curve used to evaluate how well the predicted efficacy score memb(meanOD)-cyto(meanOD)_q85 distinguished between responsive and non-responsive patients. FIG. 45 illustrates that the optimal difference threshold is 23, and the area under curve (AUC) is 0.8289. The sensitivity is 0.7647, the specificity is 0.9091, and the Youden index value is 0.6738.

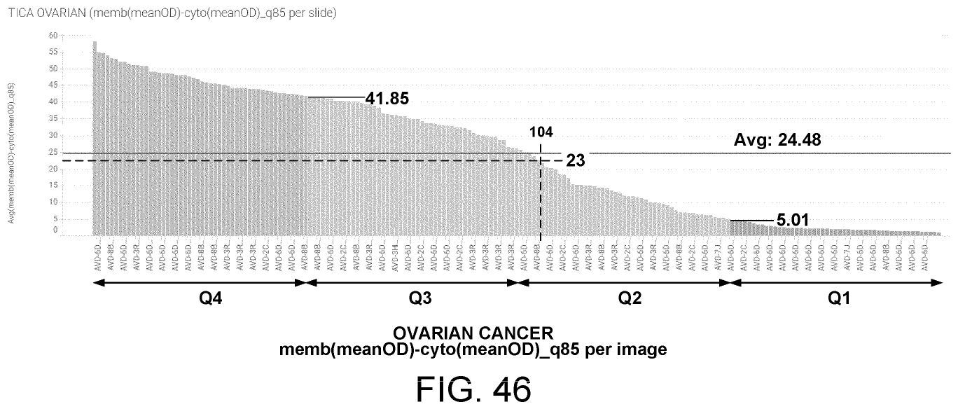

FIG. 46 shows the distribution of values of the predicted efficacy score memb(meanOD)-cyto(meanOD)_q85 in 197 tissue samples from a database of ovarian cancer patients. Stained tissue samples of the human patients were analyzed in FIG. 46 as opposed to tissue from the PDX models shown in FIG. 44. The bar graph of decreasing score values indicates the prevalence among ovarian cancer patients of various ranges of the predicted efficacy score. One quarter of the patients had a score of 5.01 or less. One quarter of the patients had a score of 41.85 or more. The average score was 24.48. FIG. 46 indicates that the tissue samples from just over half (104) of the 197 ovarian cancer patients resulted in a memb(meanOD)-cyto(meanOD)_q85 score of 23 or greater. These ovarian cancer patients would likely have a positive response to a therapy involving an anti-FRα antibody-drug conjugate.

In step 62 of method 55 of FIG. 42, a therapy involving the anti-FRα ADC is recommended to the cancer patient if, for the patient's tissue sample, the 85% quantile of the difference between membrane optical density and cytoplasm optical density exceeds a predetermined difference threshold. In this example, the predetermined difference threshold is 23. Thus, the anti-FRα ADC therapy would be recommended to 104 of the 197 ovarian cancer patients.

Although the present invention has been described in connection with certain specific embodiments for instructional purposes, the present invention is not limited thereto. Various modifications, adaptations, and combinations of various features of the described embodiments can be practiced without departing from the scope of the invention as set forth in the claims.

### XII. Preferred Embodiments of the Invention

Preferred Embodiments of the Present Invention are described below and are referred to as embodiments E1 to E32.

E1. A method of generating a recommended dosage to treat a cancer patient with an antibody drug conjugate (ADC) that includes an ADC payload and an ADC antibody or antigen-binding fragment thereof that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
computing for each cancer cell a mean optical density of staining by the dye in a membrane of the cancer cell;
computing a median optical density of all cancer cells in the digital image;
determining the recommended dosage based on whether the median optical density falls below a lower optical density threshold, between the lower optical density threshold and an upper optical density threshold, or above the upper optical density threshold, wherein the recommended dosage is zero if the median optical density falls below the lower optical density threshold, a higher dosage if the median optical density falls between the lower optical density threshold and the upper optical density threshold, and a lower dosage if the median optical density falls above the upper optical density threshold, and wherein the lower optical density threshold and the upper optical density threshold are correlated to responses of a cohort of training patients treated with the ADC; and
recommending to the cancer patient a therapy involving the ADC and the recommended dosage.

E2. The method of E1, further comprising:
administering the recommended dosage of the therapy involving the ADC to the cancer patient.

E3. The method of E1, wherein the lower optical density threshold is in a range of 21 to 29 on a scale with a maximum optical density of 220.

E4. The method of any of E1-E3, wherein the upper optical density threshold is in a range of 35 to 43 on a scale with a maximum optical density of 220.

E5. The method of any of E1-E4, wherein the lower dosage is 2.5 mg/kg or below, and wherein the higher dosage is 5 mg/kg or above.

E6. A method of generating a response score to predict a response of a cancer patient to an antibody drug conjugate (ADC) that includes an ADC payload and an ADC antibody or antigen-binding fragment thereof that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
computing for each cancer cell a mean optical density of staining by the dye in a membrane of the cancer cell;
computing a median optical density of all cancer cells in the digital image;
generating a response score for the tissue sample based on the median optical density, wherein the response score is positive if the median optical density is equal to or greater than an optical density threshold and negative if the median optical density is less than the optical density threshold, and wherein the optical density threshold is correlated to responses of a cohort of training patients treated with the ADC; and
recommending a therapy involving the ADC to the cancer patient if the response score is positive.

E7. A method of generating a response score to predict a response of a cancer patient to an antibody drug conjugate (ADC) that includes an ADC payload and an ADC antibody that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell an optical density of staining of the dye in the membrane of the cancer cell;
identifying each cancer cell as either optical-density positive if the optical density of the cancer cell is equal to or greater than an optical density threshold or optical-density negative if the mean optical density of the cancer cell is less than the optical density threshold;
generating a response score for the tissue sample based on a percentage of cancer cells in the digital image that are optical-density positive, wherein the response score is positive if the percentage of cancer cells that are optical-density positive is equal to or greater than a percentage threshold and negative if the percentage of cancer cells that are optical-density positive is less than the percentage threshold, and wherein the optical density threshold and the percentage threshold are correlated to responses of a cohort of training patients treated with the ADC; and
recommending a therapy involving the ADC to the cancer patient if the response score is positive.

E8. The method of E6 or E7, further comprising:
administering the therapy involving the ADC to the cancer patient if the response score is positive.

E9. The method of any of E6-E8, wherein the percentage threshold is in a range of 80% to 90%.

E10. A method of identifying a cancer patient for treatment with an antibody drug conjugate (ADC) that includes an ADC payload and an ADC antibody that targets a protein on cancer cells, wherein the protein is folate receptor alpha (FRα), comprising:
staining a tissue sample from the cancer patient immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
computing for each cancer cell a mean optical density of staining by the dye in a membrane of the cancer cell;
identifying each cancer cell as either optical-density positive if the mean optical density of the cancer cell is equal to or greater than an optical density threshold or optical-density negative if the mean optical density of the cancer cell is less than the optical density threshold;
generating a proximity score for the tissue sample equaling a percentage of cancer cells in the digital image that are either optical-density positive or optical-density negative but within a predefined distance of an optical-density positive cancer cell; and
identifying the cancer patient as one who will likely benefit from administration of the ADC if the proximity score exceeds a predetermined percentage threshold.

E11. The method of E10, further comprising:
recommending a therapy involving the ADC to the cancer patient when the proximity score exceeds the predetermined percentage threshold.

E12. The method of E10 or E11, further comprising:
administering the therapy involving the ADC to the cancer patient if the proximity score exceeds the predetermined percentage threshold.

E13. The method of any of E10-E12, wherein the percentage threshold is in a range of 95% to 100%.

E14. The method of any of E7-E13, wherein the optical density threshold is in a range of 10 to 15 on a scale with a maximum optical density of 220.

E15. A method of generating a response score to predict a response of a cancer patient to an antibody drug conjugate (ADC) that includes an ADC payload and an ADC antibody that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
computing for each cancer cell a mean optical density of staining by the dye in a membrane of the cancer cell;
computing a median optical density of all cancer cells in the digital image;
computing a median absolute deviation of the optical densities of the cancer cells from the median optical density of all cancer cells in the digital image;
generating a response score for the tissue sample based on the median absolute deviation, wherein the response score is positive if the median absolute deviation is equal to or greater than an deviation threshold and negative if the median absolute deviation is less than the deviation threshold, and wherein the deviation threshold is correlated to responses of a cohort of training patients treated with the ADC; and
recommending a therapy involving the ADC to the cancer patient if the response score is positive.

E16. The method of E15, further comprising:
administering the therapy involving the ADC to the cancer patient if the response score is positive.

E17. The method of E15 or E16, wherein the deviation threshold is in a range of 10 to 15 on a scale of optical density having a maximum of 220.

E18. The method of any of E7-E17, wherein the detecting of cancer cells involves detecting for each cancer cell the pixels that belong to the membrane using a cell center determined for each cancer cell.

E19. The method of any of E1-E18, wherein the staining intensity of each membrane is computed based on an average optical density of a brown diaminobenzidine (DAB) signal in pixels of the membrane.

E20. The method of any of E1-E19, wherein the ADC antibody is a humanized IgG1 monoclonal antibody.

E21. The method of any of E1-E20, wherein the dye is 3,3′-Diaminobenzidine (DAB).

E22. The method of any of E1-E21, wherein the cancer patient has a cancer selected from the group consisting of: ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

E23. The method of any of E1-E22, wherein the cancer patient has a cancer selected from the group consisting of: ovarian cancer, non-small cell lung cancer (NSCLC) and breast cancer.

E24. The method of E23, wherein the NSCLC is a selected from the group consisting of: squamous NSCLC, adenocarcinoma NSCLC, and a combination squamous NSCLC and adenocarcinoma NSCLC.

E25. The method of any of E1-E24, wherein the ADC payload is a cytotoxin.

E26. The method of E25, wherein the cytotoxin is a topoisomerase I inhibitor.

E27. The method of E26, wherein the topoisomerase I inhibitor is represented by the following formula:

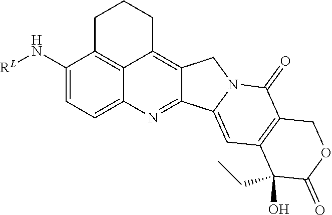

and the salts and solvates thereof, and wherein RL represents a linker connected to the ADC antibody or antigen-binding fragment thereof.

E28. The method of any of E1-E27, wherein the ADC is an anti-FRα antibody or antigen-binding fragment thereof conjugated to a topoisomerase I inhibitor, wherein the topoisomerase I inhibitor is selected from:

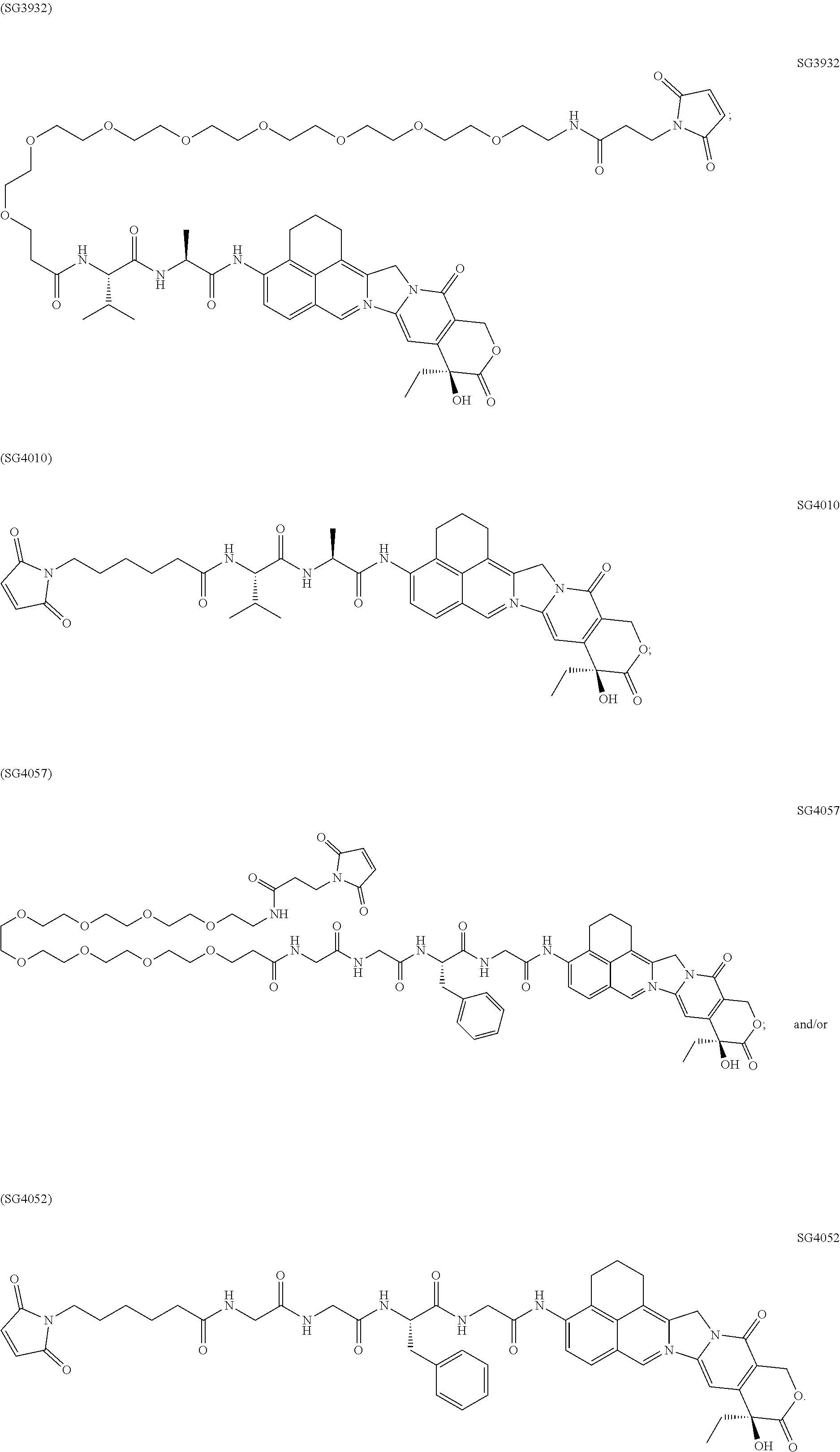

E29. The method of any of E1-E28, wherein the ADC is an anti-FRα antibody or antigen-binding fragment thereof conjugated to a topoisomerase I inhibitor, wherein the topoisomerase I inhibitor is

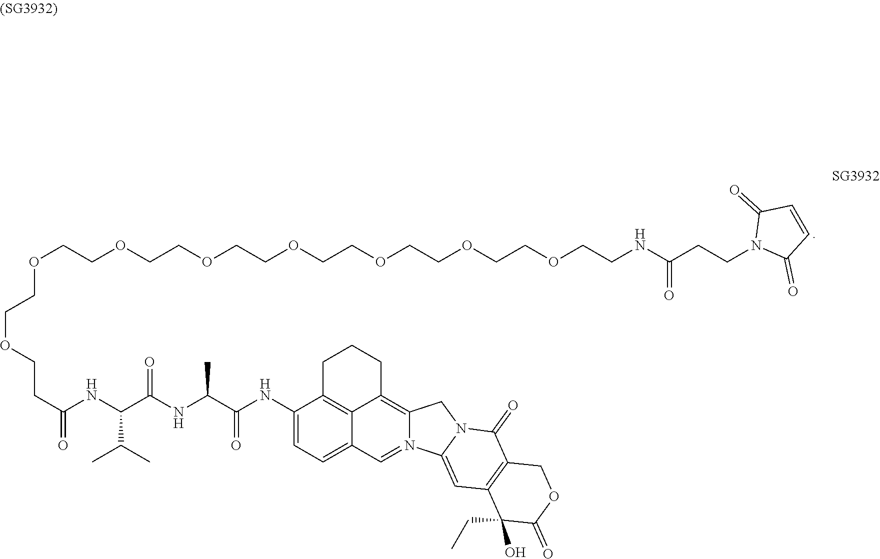

E30. The method of any of E1-E29, wherein the ADC has a drug-to-antibody ratio (DAR) that falls within a range selected from the group consisting of: 1 to 20, 1 to 10, 2 to 10, 2 to 8, 2 to 6, and 4 to 10.

E31. The method of any of E1-E30, wherein the ADC has a drug-to-antibody ratio (DAR) selected from the group consisting of: 4 and 8.

E32. The method of any of E1-E31, wherein the ADC antibody or antigen-binding fragment thereof includes a plurality of amino acid sequences selected from the group consisting of:
(a) a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG), a heavy chain CDR3 of SEQ ID NO: 9 (EMQQLALDY), a light chain CDR1 of SEQ ID NO: 10 (RASQGISNFLA), a light chain CDR2 of SEQ ID NO: 11 (AASSLQS), and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS), a heavy chain CDR3 of SEQ ID NO: 15 (GVGRFDS), a light chain CDR1 of SEQ ID NO: 16 (RASQSISSWLA), a light chain CDR2 of SEQ ID NO: 17 (KASSLES), and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG), a heavy chain CDR3 of SEQ ID NO: 21 (GMTTLTFDY), a light chain CDR1 of SEQ ID NO: 22 (RASQGISTFLA), a light chain CDR2 of SEQ ID NO: 23 (AASSLQS), and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG), a heavy chain CDR3 of SEQ ID NO: 27 (GMTTLTFDY), a light chain CDR1 of SEQ ID NO: 28 (RASQGISSFLA), a light chain CDR2 of SEQ ID NO: 29 (AASSLQS), and a light chain CDR3 of SEQ ID NO: 30 (QQYNSYPLT); and
(f) a heavy chain CDR1 of SEQ ID NO: 31 (SDSATWN), a heavy chain CDR2 of SEQ ID NO: 32 (RTYYRSKWYSDYAVSVKS), a heavy chain CDR3 of SEQ ID NO: 33 (GGAPFDY), a light chain CDR1 of SEQ ID NO: 34 (RASQSISSWLA), a light chain CDR2 of SEQ ID NO: 35 (KASSLES), and a light chain CDR3 of SEQ ID NO: 36 (QQYNSYSMYT).

E33. The method of any of E1-E32, wherein the ADC antibody or antigen-binding fragment thereof includes a plurality of amino acid sequences selected from the group consisting of:
(a) a VH chain comprising the amino acid sequence of SEQ ID NO: 37 and a VL chain comprising the amino acid sequence of SEQ ID NO: 38;
(b) a VH chain comprising the amino acid sequence of SEQ ID NO: 39 and a VL chain comprising the amino acid sequence of SEQ ID NO: 40;
(c) a VH chain comprising the amino acid sequence of SEQ ID NO: 41 and a VL chain comprising the amino acid sequence of SEQ ID NO: 42;
(d) a VH chain comprising the amino acid sequence of SEQ ID NO: 43 and a VL chain comprising the amino acid sequence of SEQ ID NO: 44;
(e) a VH chain comprising the amino acid sequence of SEQ ID NO: 45 and a VL chain comprising the amino acid sequence of SEQ ID NO: 46; and
(f) a VH chain comprising the amino acid sequence of SEQ ID NO: 47 and a VL chain comprising the amino acid sequence of SEQ ID NO: 48.

E34. The method of any of E1-E33, wherein the ADC antibody or antigen-binding fragment thereof includes a plurality of amino acid sequences selected from the group consisting of:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; and
(f) a heavy chain amino acid sequence of SEQ ID NO: 59 and a light chain amino acid sequence of SEQ ID NO: 60.

E35. The method of any of E1-E34,
wherein the ADC antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT),
wherein the ADC payload is a topoisomerase I inhibitor

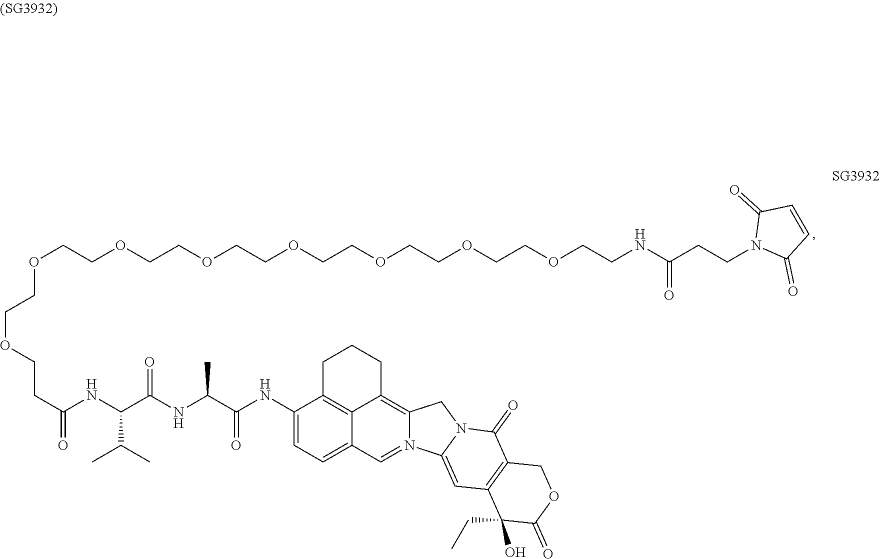

and
wherein the ADC has a drug-to-antibody ratio (DAR) of 8.

E36. The method of E35, wherein the ADC antibody or antigen-binding fragment thereof includes a VH chain comprising the amino acid sequence of SEQ ID NO: 37 and a VL chain comprising the amino acid sequence of SEQ ID NO: 38.

What is claimed is:

1. A method of determining a recommended dosage to treat a cancer patient with an antibody drug conjugate (ADC) that includes an ADC payload and an ADC antibody or antigen-binding fragment thereof that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell a mean optical density of staining by the dye in a membrane of the cancer cell;
determining a median optical density of all cancer cells in the digital image;
determining the recommended dosage based on whether the median optical density falls below a lower optical density threshold, between the lower optical density threshold and an upper optical density threshold, or above the upper optical density threshold, wherein the recommended dosage is zero if the median optical density falls below the lower optical density threshold, a higher dosage if the median optical density falls between the lower optical density threshold and the upper optical density threshold, and a lower dosage if the median optical density falls above the upper optical density threshold, and wherein the lower optical density threshold and the upper optical density threshold are correlated to responses of a cohort of training patients treated with the ADC; and
recommending to the cancer patient a therapy involving the ADC and the recommended dosage.

2. The method of claim 1, further comprising:
administering the recommended dosage of the therapy involving the ADC to the cancer patient.

3. The method of claim 1, wherein the lower optical density threshold is in a range of 21 to 29 on a scale with a maximum optical density of 220.

4. The method of claim 1, wherein the upper optical density threshold is in a range of 35 to 43 on a scale with a maximum optical density of 220.

5. The method of claim 1, wherein the lower dosage is in a range of 2.4 to 2.6 mg/kg, and wherein the higher dosage is in a range of 4.9 to 5.1 mg/kg.

6. The method of claim 1, wherein the detecting of cancer cells involves detecting for each cancer cell the pixels that belong to the membrane using a cell center determined for each cancer cell.

7. The method of claim 1, wherein the staining intensity of each membrane is computed based on an average optical density of a brown diaminobenzidine (DAB) signal in pixels of the membrane.

8. The method of claim 1, wherein the ADC antibody is a humanized IgG1 monoclonal antibody.

9. The method of claim 1, wherein the dye is 3,3′-Diaminobenzidine (DAB).

10. The method of claim 1, wherein the cancer patient has a cancer selected from the group consisting of: ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

11. The method of claim 1, wherein the cancer patient has a cancer selected from the group consisting of: ovarian cancer, non-small cell lung cancer (NSCLC) and breast cancer.

12. The method of claim 11, wherein the NSCLC is a selected from the group consisting of: squamous NSCLC, adenocarcinoma NSCLC, and a combination squamous NSCLC and adenocarcinoma NSCLC.

13. The method of claim 1, wherein the ADC payload is a cytotoxin.

14. The method of claim 13, wherein the cytotoxin is a topoisomerase I inhibitor.

15. The method of claim 14, wherein the topoisomerase I inhibitor is represented by the following formula: 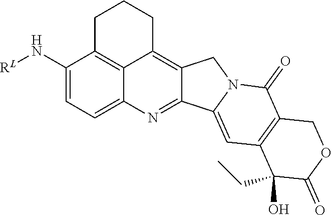
and the salts and solvates thereof, and wherein R^{L} represents a linker connected to the ADC antibody or antigen-binding fragment thereof.

16. The method of claim 1, wherein the ADC is an anti-FRα antibody or antigen-binding fragment thereof conjugated to a topoisomerase I inhibitor, wherein the topoisomerase I inhibitor is selected from:

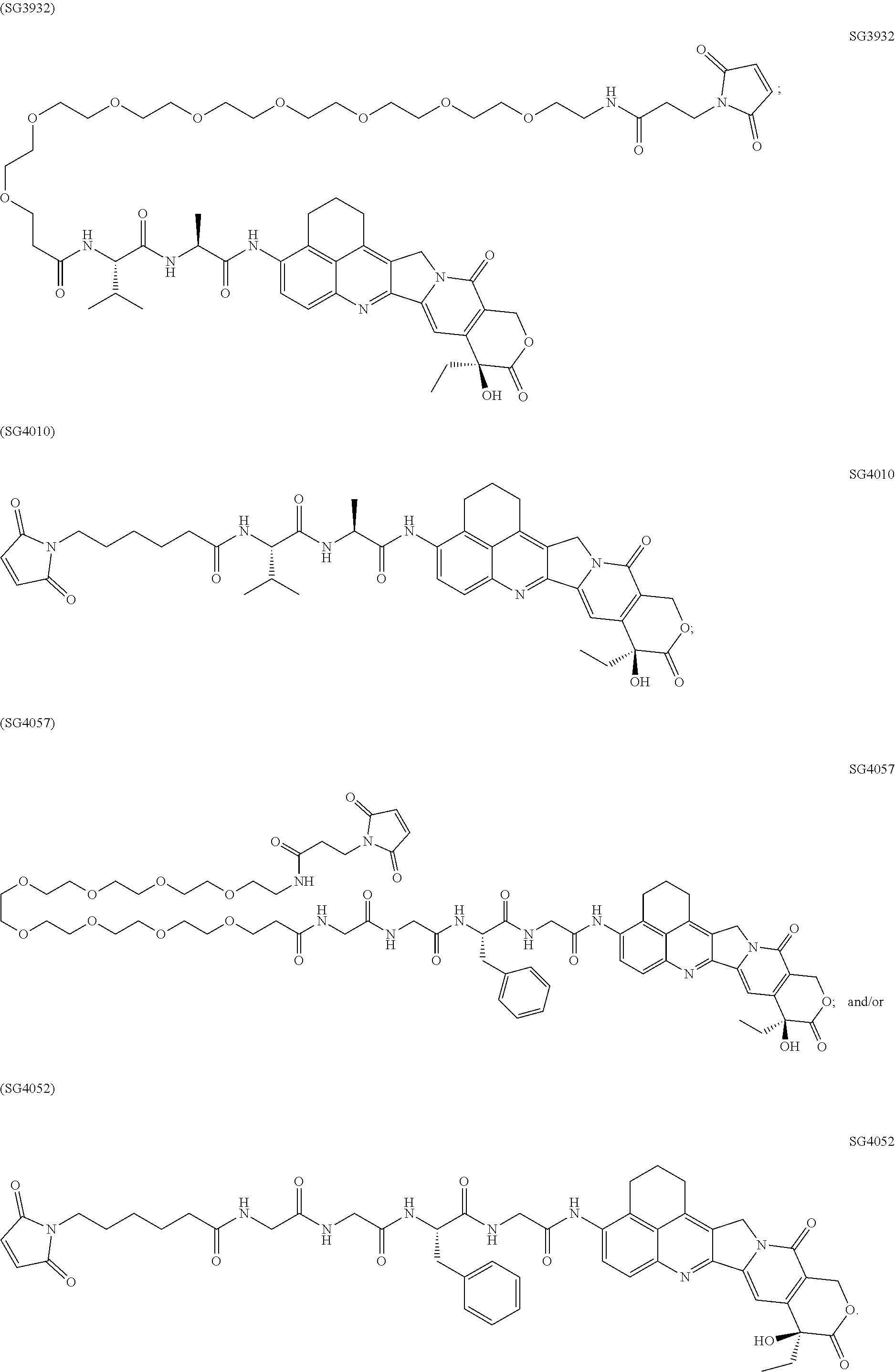

17. The method of claim 1, wherein the ADC is an anti-FRα antibody or antigen-binding fragment thereof conjugated to a topoisomerase I inhibitor, wherein the topoisomerase I inhibitor is

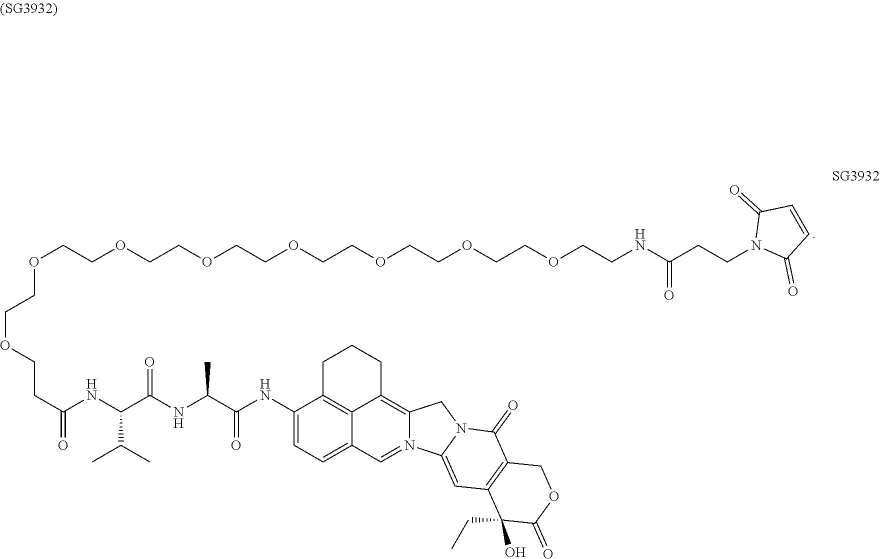

18. The method of claim 1, wherein the ADC has a drug-to-antibody ratio (DAR) that falls within a range selected from the group consisting of: 1 to 20, 1 to 10, 2 to 10, 2 to 8, 2 to 6, and 4 to 10.

19. The method of claim 1, wherein the ADC has a drug-to-antibody ratio (DAR) selected from the group consisting of: 4 and 8.

20. The method of claim 1, wherein the ADC antibody or antigen-binding fragment thereof includes a plurality of amino acid sequences selected from the group consisting of:
(a) a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG), a heavy chain CDR3 of SEQ ID NO: 9 (EMQQLALDY), a light chain CDR1 of SEQ ID NO: 10 (RASQGISNFLA), a light chain CDR2 of SEQ ID NO: 11 (AASSLQS), and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS), a heavy chain CDR3 of SEQ ID NO: 15 (GVGRFDS), a light chain CDR1 of SEQ ID NO: 16 (RASQSISSWLA), a light chain CDR2 of SEQ ID NO: 17 (KASSLES), and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG), a heavy chain CDR3 of SEQ ID NO: 21 (GMTTLTFDY), a light chain CDR1 of SEQ ID NO: 22 (RASQGISTFLA), a light chain CDR2 of SEQ ID NO: 23 (AASSLQS), and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG), a heavy chain CDR3 of SEQ ID NO: 27 (GMTTLTFDY), a light chain CDR1 of SEQ ID NO: 28 (RASQGISSFLA), a light chain CDR2 of SEQ ID NO: 29 (AASSLQS), and a light chain CDR3 of SEQ ID NO: 30 (QQYNSYPLT); and
(f) a heavy chain CDR1 of SEQ ID NO: 31 (SDSATWN), a heavy chain CDR2 of SEQ ID NO: 32 (RTYYRSKWYSDYAVSVKS), a heavy chain CDR3 of SEQ ID NO: 33 (GGAPFDY), a light chain CDR1 of SEQ ID NO: 34 (RASQSISSWLA), a light chain CDR2 of SEQ ID NO: 35 (KASSLES), and a light chain CDR3 of SEQ ID NO: 36 (QQYNSYSMYT).

21. The method of claim 1, wherein the ADC antibody or antigen-binding fragment thereof includes a plurality of amino acid sequences selected from the group consisting of:
(a) a VH chain comprising the amino acid sequence of SEQ ID NO: 37 and a VL chain comprising the amino acid sequence of SEQ ID NO: 38;
(b) a VH chain comprising the amino acid sequence of SEQ ID NO: 39 and a VL chain comprising the amino acid sequence of SEQ ID NO: 40;
(c) a VH chain comprising the amino acid sequence of SEQ ID NO: 41 and a VL chain comprising the amino acid sequence of SEQ ID NO: 42;
(d) a VH chain comprising the amino acid sequence of SEQ ID NO: 43 and a VL chain comprising the amino acid sequence of SEQ ID NO: 44;
(e) a VH chain comprising the amino acid sequence of SEQ ID NO: 45 and a VL chain comprising the amino acid sequence of SEQ ID NO: 46; and
(f) a VH chain comprising the amino acid sequence of SEQ ID NO: 47 and a VL chain comprising the amino acid sequence of SEQ ID NO: 48.

22. The method of claim 1, wherein the ADC antibody or antigen-binding fragment thereof includes a plurality of amino acid sequences selected from the group consisting of:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; and
(f) a heavy chain amino acid sequence of SEQ ID NO: 59 and a light chain amino acid sequence of SEQ ID NO: 60.

23. The method of claim 1,
wherein the ADC antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT),
wherein the ADC payload is a topoisomerase I inhibitor 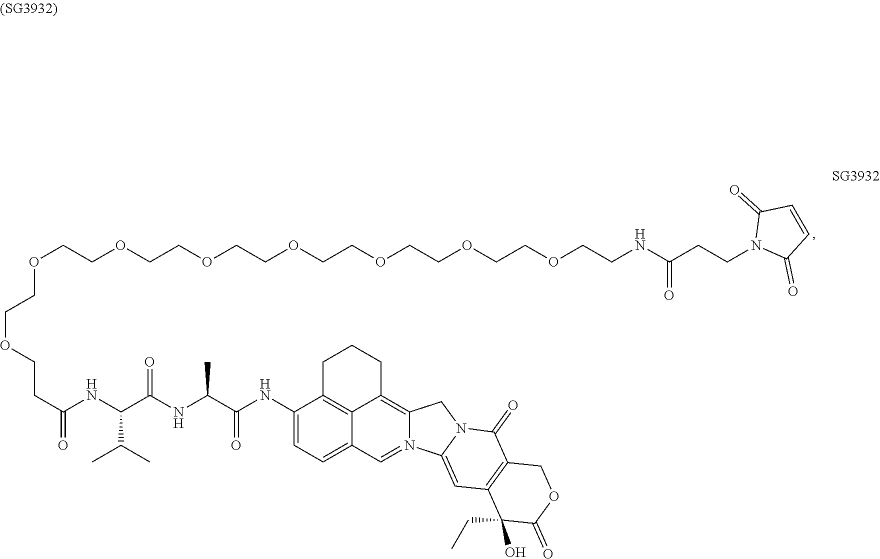
and
wherein the ADC has a drug-to-antibody ratio (DAR) of 8.

24. The method of claim 23, wherein the ADC antibody or antigen-binding fragment thereof includes a VH chain comprising the amino acid sequence of SEQ ID NO: 37 and a VL chain comprising the amino acid sequence of SEQ ID NO: 38.

25. A method of generating a predicted efficacy score to predict a response of a cancer patient to an antibody drug conjugate (ADC) that includes an ADC payload and an ADC antibody or antigen-binding fragment thereof that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell a mean optical density of staining by the dye in a membrane of the cancer cell;
determining a median optical density of all cancer cells in the digital image;
generating a predicted efficacy score for the tissue sample based on the median optical density, wherein the predicted efficacy score is positive if the median optical density is equal to or greater than an optical density threshold and negative if the median optical density is less than the optical density threshold, and wherein the optical density threshold is correlated to responses of a cohort of training patients treated with the ADC; and
recommending a therapy involving the ADC to the cancer patient if the predicted efficacy score is positive.

26. The method of claim 25, further comprising:
administering the therapy involving the ADC to the cancer patient when the predicted efficacy score is positive.

27. The method of claim 25, wherein the detecting of cancer cells involves detecting for each cancer cell the pixels that belong to the membrane using a cell center determined for each cancer cell.

28. The method of claim 25, wherein the staining intensity of each membrane is computed based on an average optical density of a brown diaminobenzidine (DAB) signal in pixels of the membrane.

29. The method of claim 25, wherein the ADC antibody is a humanized IgG1 monoclonal antibody.

30. The method of claim 25, wherein the dye is 3,3′-Diaminobenzidine (DAB).

31. The method of claim 25, wherein the cancer patient has a cancer selected from the group consisting of: ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

32. The method of claim 25, wherein the cancer patient has a cancer selected from the group consisting of: ovarian cancer, non-small cell lung cancer (NSCLC) and breast cancer.

33. The method of claim 32, wherein the NSCLC is a selected from the group consisting of: squamous NSCLC, adenocarcinoma NSCLC, and a combination squamous NSCLC and adenocarcinoma NSCLC.

34. The method of claim 25, wherein the ADC payload is a cytotoxin.

35. The method of claim 34, wherein the cytotoxin is a topoisomerase I inhibitor.

36. The method of claim 35, wherein the topoisomerase I inhibitor is represented by the following formula: 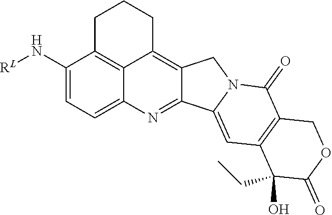
and the salts and solvates thereof, and wherein R^{L} represents a linker connected to the ADC antibody or antigen-binding fragment thereof.

37. The method of claim 25, wherein the ADC is an anti-FRα antibody or antigen-binding fragment thereof conjugated to a topoisomerase I inhibitor, wherein the topoisomerase I inhibitor is selected from:

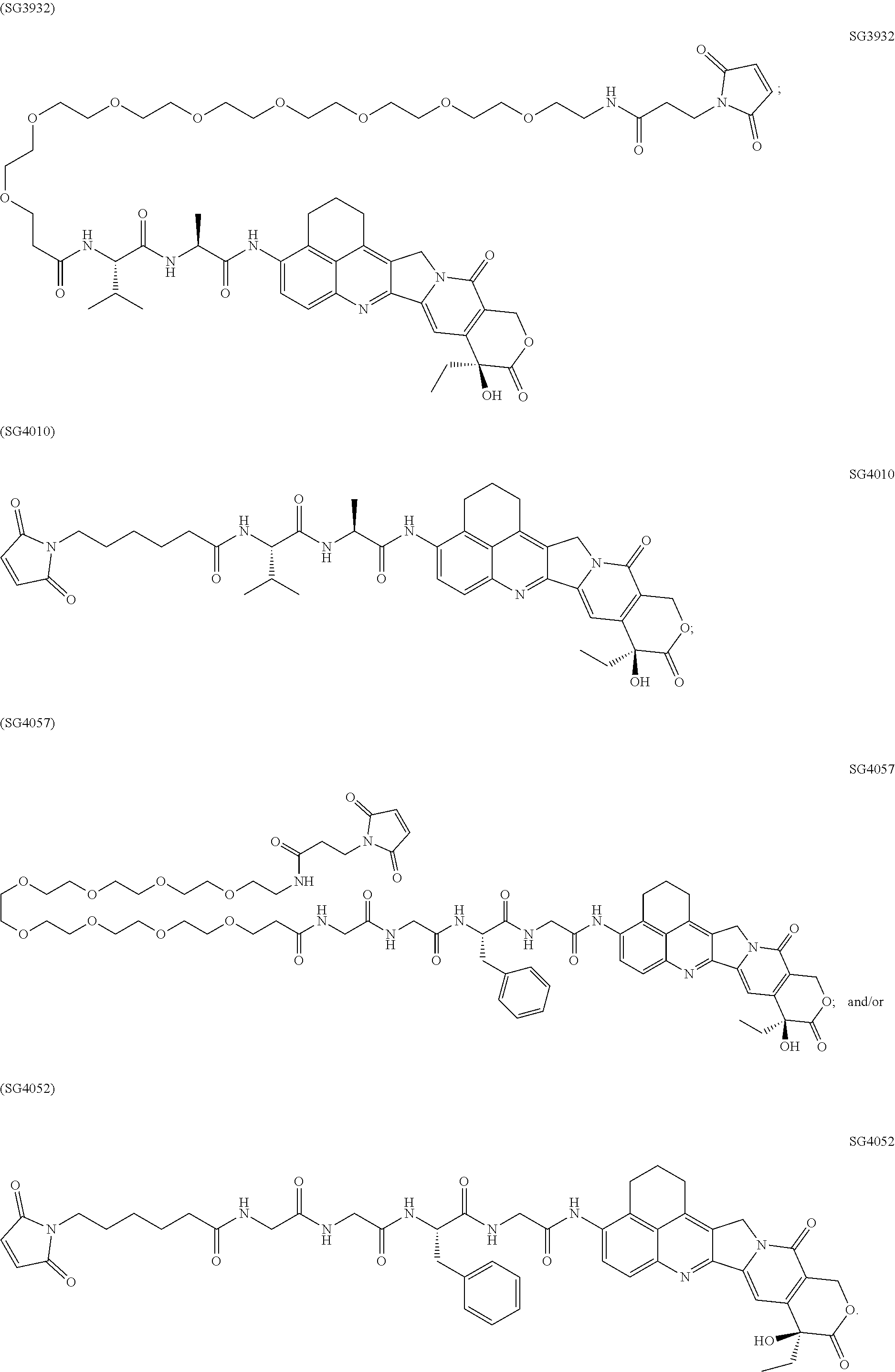

38. The method of claim 25, wherein the ADC is an anti-FRα antibody or antigen-binding fragment thereof conjugated to a topoisomerase I inhibitor, wherein the topoisomerase I inhibitor is

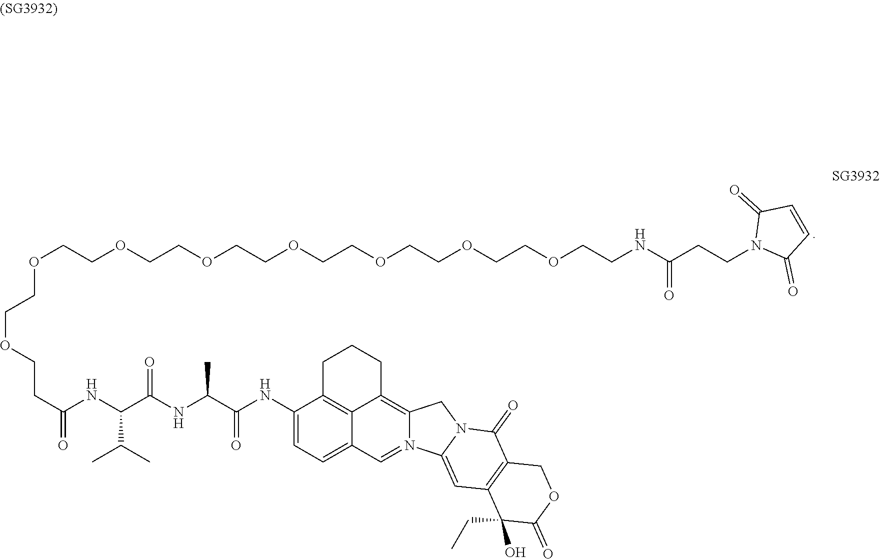

39. The method of claim 25, wherein the ADC has a drug-to-antibody ratio (DAR) that falls within a range selected from the group consisting of: 1 to 20, 1 to 10, 2 to 10, 2 to 8, 2 to 6, and 4 to 10.

40. The method of claim 25, wherein the ADC has a drug-to-antibody ratio (DAR) selected from the group consisting of: 4 and 8.

41. The method of claim 25, wherein the ADC antibody or antigen-binding fragment thereof includes a plurality of amino acid sequences selected from the group consisting of:
(a) a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG), a heavy chain CDR3 of SEQ ID NO: 9 (EMQQLALDY), a light chain CDR1 of SEQ ID NO: 10 (RASQGISNFLA), a light chain CDR2 of SEQ ID NO: 11 (AASSLQS), and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS), a heavy chain CDR3 of SEQ ID NO: 15 (GVGRFDS), a light chain CDR1 of SEQ ID NO: 16 (RASQSISSWLA), a light chain CDR2 of SEQ ID NO: 17 (KASSLES), and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG), a heavy chain CDR3 of SEQ ID NO: 21 (GMTTLTFDY), a light chain CDR1 of SEQ ID NO: 22 (RASQGISTFLA), a light chain CDR2 of SEQ ID NO: 23 (AASSLQS), and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG), a heavy chain CDR3 of SEQ ID NO: 27 (GMTTLTFDY), a light chain CDR1 of SEQ ID NO: 28 (RASQGISSFLA), a light chain CDR2 of SEQ ID NO: 29 (AASSLQS), and a light chain CDR3 of SEQ ID NO: 30 (QQYNSYPLT); and
(f) a heavy chain CDR1 of SEQ ID NO: 31 (SDSATWN), a heavy chain CDR2 of SEQ ID NO: 32 (RTYYRSKWYSDYAVSVKS), a heavy chain CDR3 of SEQ ID NO: 33 (GGAPFDY), a light chain CDR1 of SEQ ID NO: 34 (RASQSISSWLA), a light chain CDR2 of SEQ ID NO: 35 (KASSLES), and a light chain CDR3 of SEQ ID NO: 36 (QQYNSYSMYT).

42. The method of claim 25, wherein the ADC antibody or antigen-binding fragment thereof includes a plurality of amino acid sequences selected from the group consisting of:
(a) a VH chain comprising the amino acid sequence of SEQ ID NO: 37 and a VL chain comprising the amino acid sequence of SEQ ID NO: 38;
(b) a VH chain comprising the amino acid sequence of SEQ ID NO: 39 and a VL chain comprising the amino acid sequence of SEQ ID NO: 40;
(c) a VH chain comprising the amino acid sequence of SEQ ID NO: 41 and a VL chain comprising the amino acid sequence of SEQ ID NO: 42;
(d) a VH chain comprising the amino acid sequence of SEQ ID NO: 43 and a VL chain comprising the amino acid sequence of SEQ ID NO: 44;
(e) a VH chain comprising the amino acid sequence of SEQ ID NO: 45 and a VL chain comprising the amino acid sequence of SEQ ID NO: 46; and
(f) a VH chain comprising the amino acid sequence of SEQ ID NO: 47 and a VL chain comprising the amino acid sequence of SEQ ID NO: 48.

43. The method of claim 25, wherein the ADC antibody or antigen-binding fragment thereof includes a plurality of amino acid sequences selected from the group consisting of:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; and
(f) a heavy chain amino acid sequence of SEQ ID NO: 59 and a light chain amino acid sequence of SEQ ID NO: 60.

44. The method of claim 25,
wherein the ADC antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT),
wherein the ADC payload is a topoisomerase I inhibitor 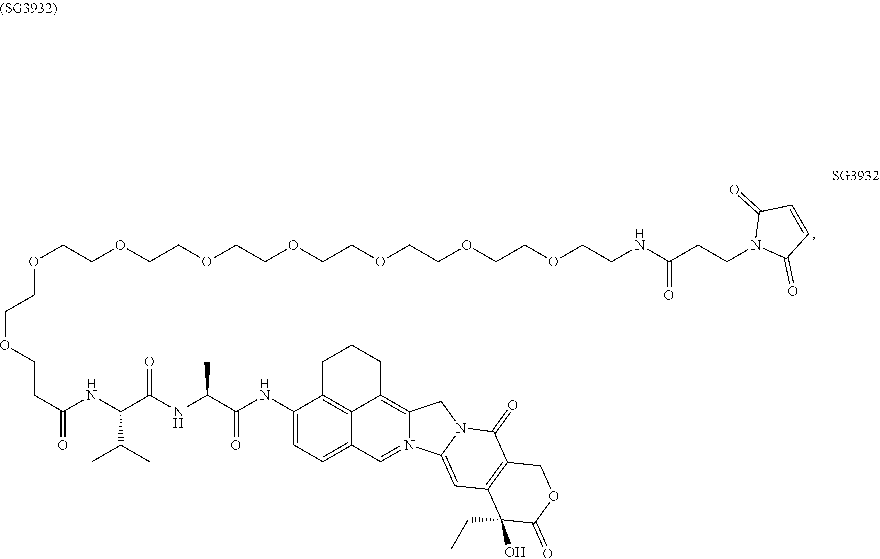
and
wherein the ADC has a drug-to-antibody ratio (DAR) of 8.

45. The method of claim 44, wherein the ADC antibody or antigen-binding fragment thereof includes a VH chain comprising the amino acid sequence of SEQ ID NO: 37 and a VL chain comprising the amino acid sequence of SEQ ID NO: 38.

46. A method of generating a predicted efficacy score to predict a response of a cancer patient to an antibody drug conjugate (ADC) that includes an ADC payload and an ADC antibody that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell an optical density of staining of the dye in the membrane of the cancer cell;
identifying each cancer cell as either optical-density positive if the optical density of the cancer cell is equal to or greater than an optical density threshold or optical-density negative if the mean optical density of the cancer cell is less than the optical density threshold;
generating a predicted efficacy score for the tissue sample based on a percentage of cancer cells in the digital image that are optical-density positive, wherein the predicted efficacy score is positive if the percentage of cancer cells that are optical-density positive is equal to or greater than a percentage threshold and negative if the percentage of cancer cells that are optical-density positive is less than the percentage threshold, and wherein the optical density threshold and the percentage threshold are correlated to responses of a cohort of training patients treated with the ADC; and
recommending a therapy involving the ADC to the cancer patient if the predicted efficacy score is positive.

47. The method of claim 46, further comprising:
administering the therapy involving the ADC to the cancer patient when the predicted efficacy score is positive.

48. The method of claim 46, wherein the optical density threshold is in a range of 10 to 15 on a scale with a maximum optical density of 220.

49. The method of claim 46, wherein the percentage threshold is in a range of 80% to 90%.

50. The method of claim 46, wherein the detecting of cancer cells involves detecting for each cancer cell the pixels that belong to the membrane using a cell center determined for each cancer cell.

51. The method of claim 46, wherein the staining intensity of each membrane is computed based on an average optical density of a brown diaminobenzidine (DAB) signal in pixels of the membrane.

52. The method of claim 46, wherein the ADC antibody is a humanized IgG1 monoclonal antibody.

53. The method of claim 46, wherein the dye is 3,3′-Diaminobenzidine (DAB).

54. The method of claim 46, wherein the cancer patient has a cancer selected from the group consisting of: ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

55. The method of claim 46, wherein the cancer patient has a cancer selected from the group consisting of: ovarian cancer, non-small cell lung cancer (NSCLC) and breast cancer.

56. The method of claim 55, wherein the NSCLC is a selected from the group consisting of: squamous NSCLC, adenocarcinoma NSCLC, and a combination squamous NSCLC and adenocarcinoma NSCLC.

57. The method of claim 46, wherein the ADC payload is a cytotoxin.

58. The method of claim 57, wherein the cytotoxin is a topoisomerase I inhibitor.

59. The method of claim 58, wherein the topoisomerase I inhibitor is represented by the following formula: 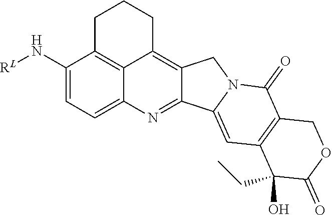
and the salts and solvates thereof, and wherein R^{L} represents a linker connected to the ADC antibody or antigen-binding fragment thereof.

60. The method of claim 46, wherein the ADC is an anti-FRα antibody or antigen-binding fragment thereof conjugated to a topoisomerase I inhibitor, wherein the topoisomerase I inhibitor is selected from:

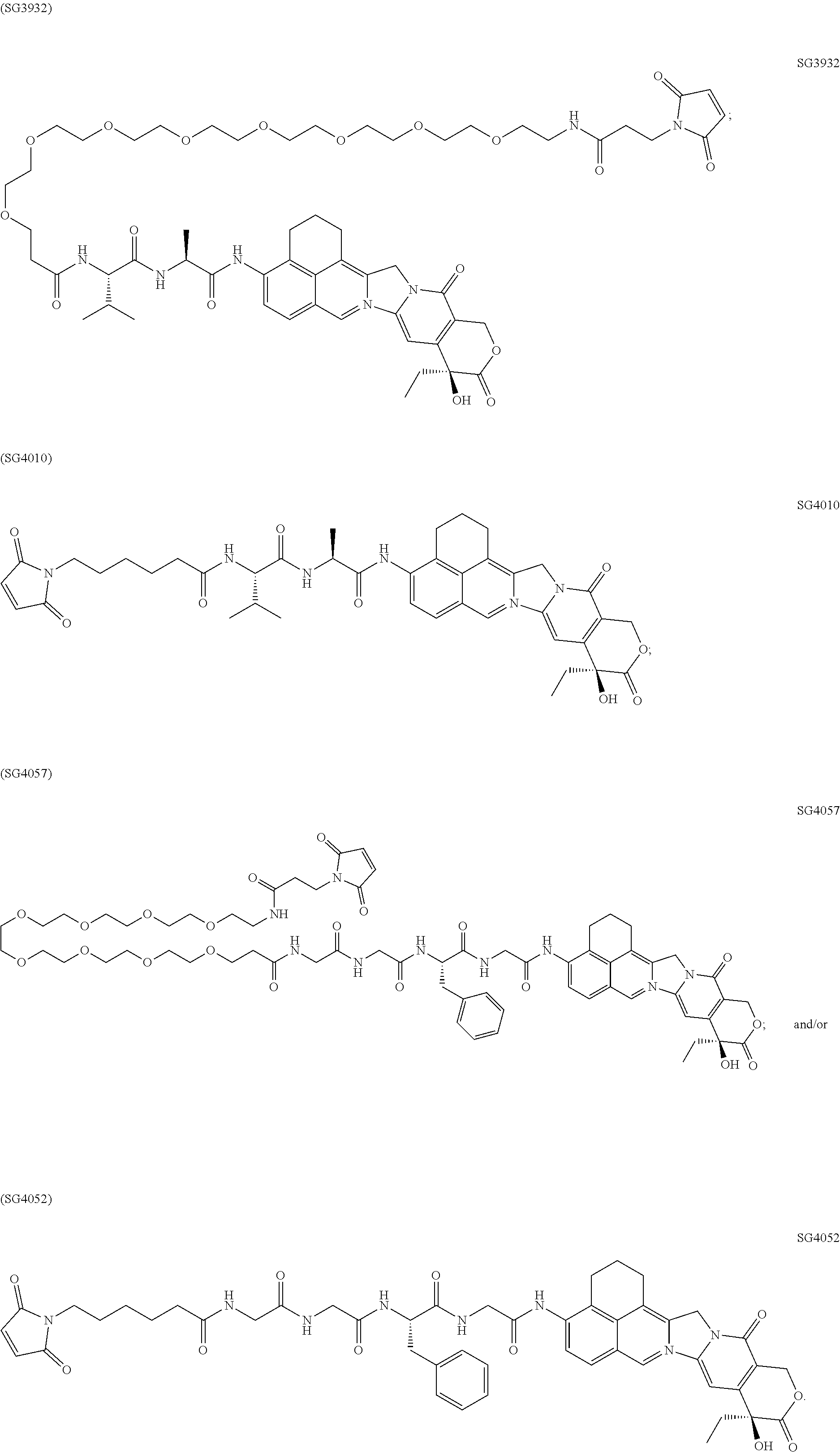

61. The method of claim 46, wherein the ADC is an anti-FRα antibody or antigen-binding fragment thereof conjugated to a topoisomerase I inhibitor, wherein the topoisomerase I inhibitor is

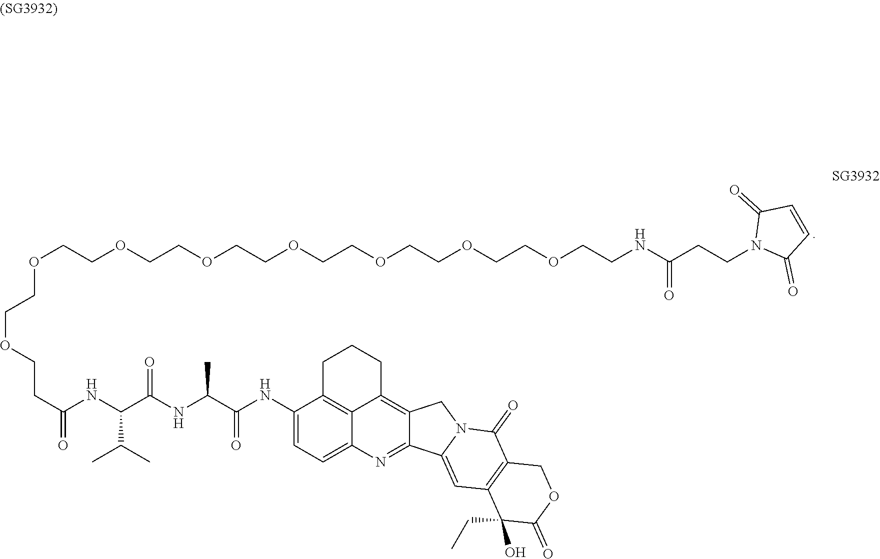 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

62. The method of claim 46, wherein the ADC has a drug-to-antibody ratio (DAR) that falls within a range selected from the group consisting of: 1 to 20, 1 to 10, 2 to 10, 2 to 8, 2 to 6, and 4 to 10.

63. The method of claim 46, wherein the ADC has a drug-to-antibody ratio (DAR) selected from the group consisting of: 4 and 8.

64. The method of claim 46, wherein the ADC antibody or antigen-binding fragment thereof includes a plurality of amino acid sequences selected from the group consisting of:
(a) a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG), a heavy chain CDR3 of SEQ ID NO: 9 (EMQQLALDY), a light chain CDR1 of SEQ ID NO: 10 (RASQGISNFLA), a light chain CDR2 of SEQ ID NO: 11 (AASSLQS), and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS), a heavy chain CDR3 of SEQ ID NO: 15 (GVGRFDS), a light chain CDR1 of SEQ ID NO: 16 (RASQSISSWLA), a light chain CDR2 of SEQ ID NO: 17 (KASSLES), and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG), a heavy chain CDR3 of SEQ ID NO: 21 (GMTTLTFDY), a light chain CDR1 of SEQ ID NO: 22 (RASQGISTFLA), a light chain CDR2 of SEQ ID NO: 23 (AASSLQS), and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG), a heavy chain CDR3 of SEQ ID NO: 27 (GMTTLTFDY), a light chain CDR1 of SEQ ID NO: 28 (RASQGISSFLA), a light chain CDR2 of SEQ ID NO: 29 (AASSLQS), and a light chain CDR3 of SEQ ID NO: 30 (QQYNSYPLT); and
(f) a heavy chain CDR1 of SEQ ID NO: 31 (SDSATWN), a heavy chain CDR2 of SEQ ID NO: 32 (RTYYRSKWYSDYAVSVKS), a heavy chain CDR3 of SEQ ID NO: 33 (GGAPFDY), a light chain CDR1 of SEQ ID NO: 34 (RASQSISSWLA), a light chain CDR2 of SEQ ID NO: 35 (KASSLES), and a light chain CDR3 of SEQ ID NO: 36 (QQYNSYSMYT).

65. The method of claim 46, wherein the ADC antibody or antigen-binding fragment thereof includes a plurality of amino acid sequences selected from the group consisting of:
(a) a VH chain comprising the amino acid sequence of SEQ ID NO: 37 and a VL chain comprising the amino acid sequence of SEQ ID NO: 38;
(b) a VH chain comprising the amino acid sequence of SEQ ID NO: 39 and a VL chain comprising the amino acid sequence of SEQ ID NO: 40;
(c) a VH chain comprising the amino acid sequence of SEQ ID NO: 41 and a VL chain comprising the amino acid sequence of SEQ ID NO: 42;
(d) a VH chain comprising the amino acid sequence of SEQ ID NO: 43 and a VL chain comprising the amino acid sequence of SEQ ID NO: 44;
(e) a VH chain comprising the amino acid sequence of SEQ ID NO: 45 and a VL chain comprising the amino acid sequence of SEQ ID NO: 46; and
(f) a VH chain comprising the amino acid sequence of SEQ ID NO: 47 and a VL chain comprising the amino acid sequence of SEQ ID NO: 48.

66. The method of claim 46, wherein the ADC antibody or antigen-binding fragment thereof includes a plurality of amino acid sequences selected from the group consisting of:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; and
(f) a heavy chain amino acid sequence of SEQ ID NO: 59 and a light chain amino acid sequence of SEQ ID NO: 60.

67. The method of claim 46,
wherein the ADC antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT),
wherein the ADC payload is a topoisomerase I inhibitor 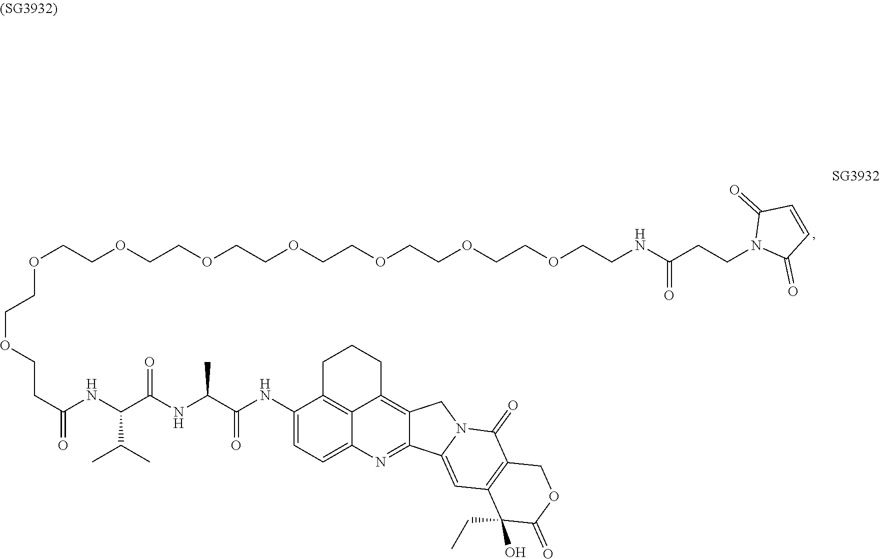 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]
and
wherein the ADC has a drug-to-antibody ratio (DAR) of 8.

68. The method of claim 67, wherein the ADC antibody or antigen-binding fragment thereof includes a VH chain comprising the amino acid sequence of SEQ ID NO: 37 and a VL chain comprising the amino acid sequence of SEQ ID NO: 38.

69. A method of identifying a cancer patient for treatment with an antibody drug conjugate (ADC) that includes an ADC payload and an ADC antibody that targets a protein on cancer cells, wherein the protein is folate receptor alpha (FRα), comprising:
staining a tissue sample from the cancer patient immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell a mean optical density of staining by the dye in a membrane of the cancer cell;
identifying each cancer cell as either optical-density positive if the mean optical density of the cancer cell is equal to or greater than an optical density threshold or optical-density negative if the mean optical density of the cancer cell is less than the optical density threshold;
generating a proximity score for the tissue sample equaling a percentage of cancer cells in the digital image that are either optical-density positive or optical-density negative but within a predefined distance of an optical-density positive cancer cell; and
identifying the cancer patient as one who will likely benefit from administration of the ADC if the proximity score exceeds a predetermined percentage threshold.

70. The method of claim 69, further comprising:
recommending a therapy involving the ADC to the cancer patient when the proximity score exceeds the predetermined percentage threshold.

71. The method of claim 69, further comprising:
administering the therapy involving the ADC to the cancer patient when the proximity score exceeds the predetermined percentage threshold.

72. The method of claim 69, wherein the optical density threshold is in a range of 10 to 15 on a scale with a maximum optical density of 220.

73. The method of claim 69, wherein the percentage threshold is in a range of 95% to 100%.

74. The method of claim 69, wherein the detecting of cancer cells involves detecting for each cancer cell the pixels that belong to the membrane using a cell center determined for each cancer cell.

75. The method of claim 69, wherein the staining intensity of each membrane is computed based on an average optical density of a brown diaminobenzidine (DAB) signal in pixels of the membrane.

76. The method of claim 69, wherein the ADC antibody is a humanized IgG1 monoclonal antibody.

77. The method of claim 69, wherein the dye is 3,3′-Diaminobenzidine (DAB).

78. The method of claim 69, wherein the cancer patient has a cancer selected from the group consisting of: ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

79. The method of claim 69, wherein the cancer patient has a cancer selected from the group consisting of: ovarian cancer, non-small cell lung cancer (NSCLC) and breast cancer.

80. The method of claim 79, wherein the NSCLC is a selected from the group consisting of: squamous NSCLC, adenocarcinoma NSCLC, and a combination squamous NSCLC and adenocarcinoma NSCLC.

81. The method of claim 69, wherein the ADC payload is a cytotoxin.

82. The method of claim 81, wherein the cytotoxin is a topoisomerase I inhibitor.

83. The method of claim 82, wherein the topoisomerase I inhibitor is represented by the following formula: 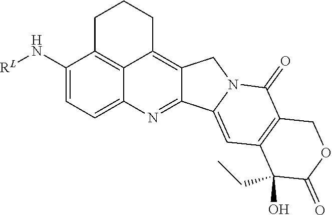
and the salts and solvates thereof, and wherein R^{L} represents a linker connected to the ADC antibody or antigen-binding fragment thereof.

84. The method of claim 69, wherein the ADC is an anti-FRα antibody or antigen-binding fragment thereof conjugated to a topoisomerase I inhibitor, wherein the topoisomerase I inhibitor is selected from:

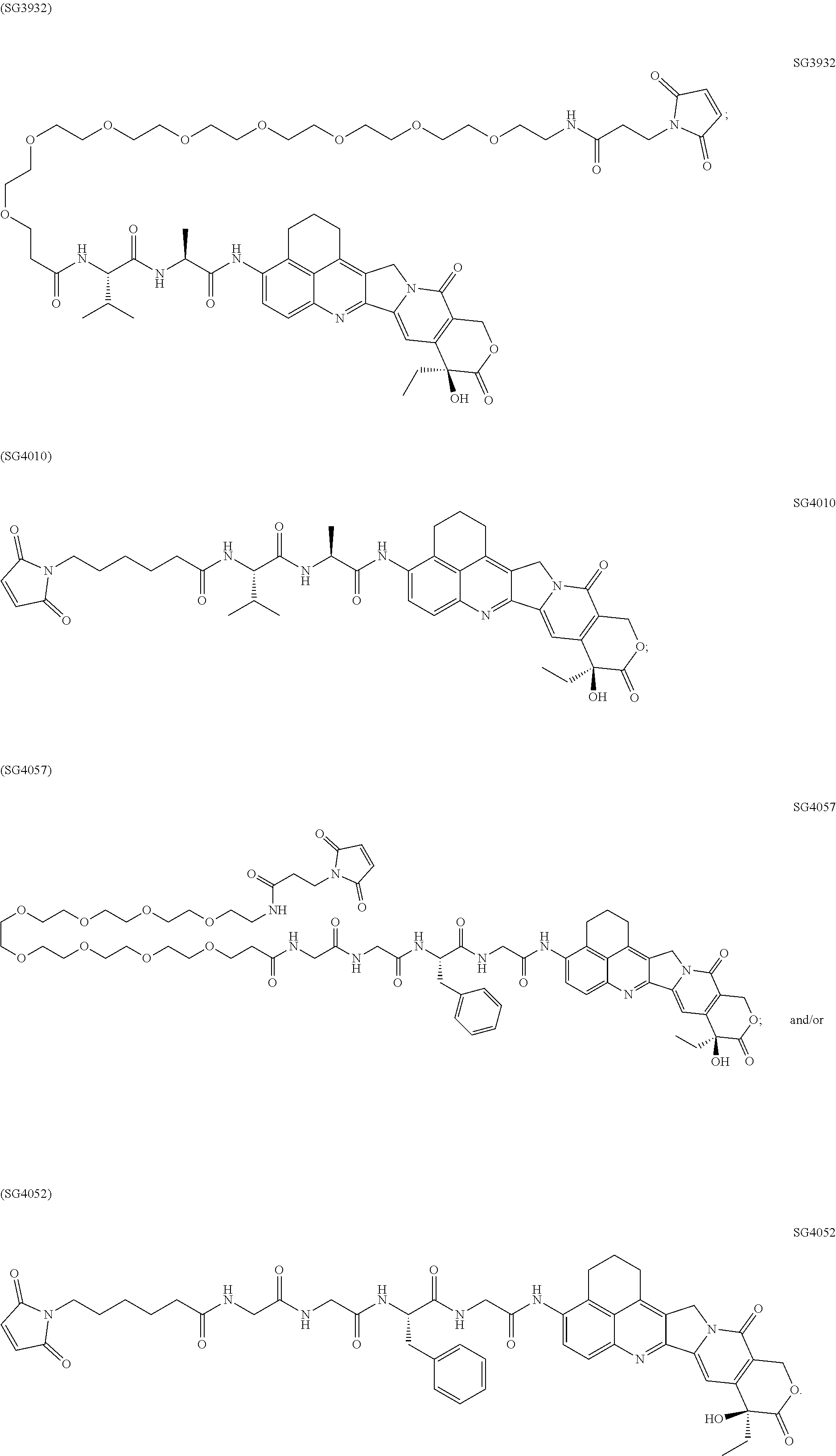

85. The method of claim 69, wherein the ADC is an anti-FRα antibody or antigen-binding fragment thereof conjugated to a topoisomerase I inhibitor, wherein the topoisomerase I inhibitor is

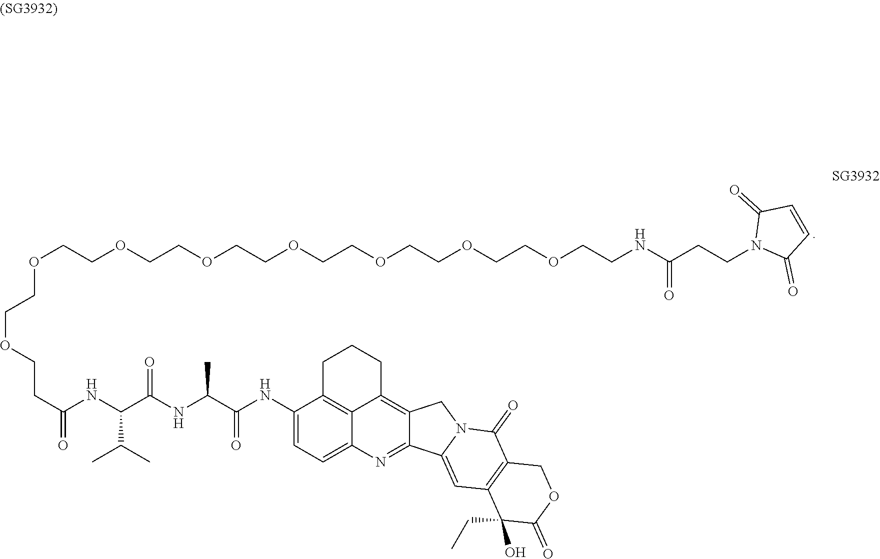 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

86. The method of claim 69, wherein the ADC has a drug-to-antibody ratio (DAR) that falls within a range selected from the group consisting of: 1 to 20, 1 to 10, 2 to 10, 2 to 8, 2 to 6, and 4 to 10.

87. The method of claim 69, wherein the ADC has a drug-to-antibody ratio (DAR) selected from the group consisting of:
4 and 8.

88. The method of claim 69, wherein the ADC antibody or antigen-binding fragment thereof includes a plurality of amino acid sequences selected from the group consisting of:
(a) a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG), a heavy chain CDR3 of SEQ ID NO: 9 (EMQQLALDY), a light chain CDR1 of SEQ ID NO: 10 (RASQGISNFLA), a light chain CDR2 of SEQ ID NO: 11 (AASSLQS), and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS), a heavy chain CDR3 of SEQ ID NO: 15 (GVGRFDS), a light chain CDR1 of SEQ ID NO: 16 (RASQSISSWLA), a light chain CDR2 of SEQ ID NO: 17 (KASSLES), and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG), a heavy chain CDR3 of SEQ ID NO: 21 (GMTTLTFDY), a light chain CDR1 of SEQ ID NO: 22 (RASQGISTFLA), a light chain CDR2 of SEQ ID NO: 23 (AASSLQS), and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG), a heavy chain CDR3 of SEQ ID NO: 27 (GMTTLTFDY), a light chain CDR1 of SEQ ID NO: 28 (RASQGISSFLA), a light chain CDR2 of SEQ ID NO: 29 (AASSLQS), and a light chain CDR3 of SEQ ID NO: 30 (QQYNSYPLT); and
(f) a heavy chain CDR1 of SEQ ID NO: 31 (SDSATWN), a heavy chain CDR2 of SEQ ID NO: 32 (RTYYRSKWYSDYAVSVKS), a heavy chain CDR3 of SEQ ID NO: 33 (GGAPFDY), a light chain CDR1 of SEQ ID NO: 34 (RASQSISSWLA), a light chain CDR2 of SEQ ID NO: 35 (KASSLES), and a light chain CDR3 of SEQ ID NO: 36 (QQYNSYSMYT).

89. The method of claim 69, wherein the ADC antibody or antigen-binding fragment thereof includes a plurality of amino acid sequences selected from the group consisting of:
(a) a VH chain comprising the amino acid sequence of SEQ ID NO: 37 and a VL chain comprising the amino acid sequence of SEQ ID NO: 38;
(b) a VH chain comprising the amino acid sequence of SEQ ID NO: 39 and a VL chain comprising the amino acid sequence of SEQ ID NO: 40;
(c) a VH chain comprising the amino acid sequence of SEQ ID NO: 41 and a VL chain comprising the amino acid sequence of SEQ ID NO: 42;
(d) a VH chain comprising the amino acid sequence of SEQ ID NO: 43 and a VL chain comprising the amino acid sequence of SEQ ID NO: 44;
(e) a VH chain comprising the amino acid sequence of SEQ ID NO: 45 and a VL chain comprising the amino acid sequence of SEQ ID NO: 46; and
(f) a VH chain comprising the amino acid sequence of SEQ ID NO: 47 and a VL chain comprising the amino acid sequence of SEQ ID NO: 48.

90. The method of claim 69, wherein the ADC antibody or antigen-binding fragment thereof includes a plurality of amino acid sequences selected from the group consisting of:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; and
(f) a heavy chain amino acid sequence of SEQ ID NO: 59 and a light chain amino acid sequence of SEQ ID NO: 60.

91. The method of claim 69,
wherein the ADC antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT),
wherein the ADC payload is a topoisomerase I inhibitor 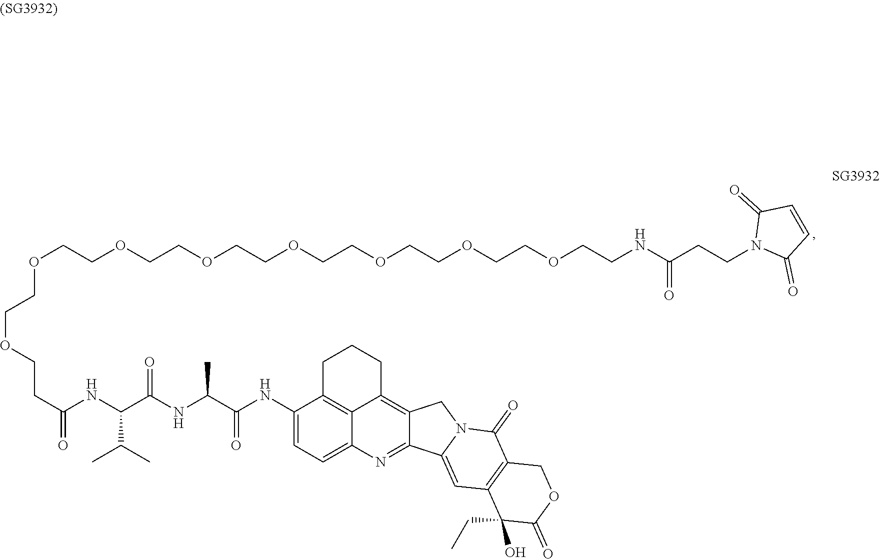 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]
and
wherein the ADC has a drug-to-antibody ratio (DAR) of 8.

92. The method of claim 91, wherein the ADC antibody or antigen-binding fragment thereof includes a VH chain comprising the amino acid sequence of SEQ ID NO: 37 and a VL chain comprising the amino acid sequence of SEQ ID NO: 38.

93. A method of generating a predicted efficacy score to predict a response of a cancer patient to an antibody drug conjugate (ADC) that includes an ADC payload and an ADC antibody that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell a mean optical density of staining by the dye in a membrane of the cancer cell;
determining a median optical density of all cancer cells in the digital image;
determining a median absolute deviation of the optical densities of the cancer cells from the median optical density of all cancer cells in the digital image;
generating a predicted efficacy score for the tissue sample based on the median absolute deviation, wherein the predicted efficacy score is positive if the median absolute deviation is equal to or greater than a deviation threshold and negative if the median absolute deviation is less than the deviation threshold, and wherein the deviation threshold is correlated to responses of a cohort of training patients treated with the ADC; and
recommending a therapy involving the ADC to the cancer patient if the predicted efficacy score is positive.

94. The method of claim 93, further comprising:
administering the therapy involving the ADC to the cancer patient when the predicted efficacy score is positive.

95. The method of claim 93, wherein the deviation threshold is in a range of 10 to 15 on a scale of optical density having a maximum of 220.

96. The method of claim 93, wherein the detecting of cancer cells involves detecting for each cancer cell the pixels that belong to the membrane using a cell center determined for each cancer cell.

97. The method of claim 93, wherein the staining intensity of each membrane is computed based on an average optical density of a brown diaminobenzidine (DAB) signal in pixels of the membrane.

98. The method of claim 93, wherein the ADC antibody is a humanized IgG1 monoclonal antibody.

99. The method of claim 93, wherein the dye is 3,3′-Diaminobenzidine (DAB).

100. The method of claim 93, wherein the cancer patient has a cancer selected from the group consisting of: ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

101. The method of claim 93, wherein the cancer patient has a cancer selected from the group consisting of: ovarian cancer, non-small cell lung cancer (NSCLC) and breast cancer.

102. The method of claim 101, wherein the NSCLC is a selected from the group consisting of: squamous NSCLC, adenocarcinoma NSCLC, and a combination squamous NSCLC and adenocarcinoma NSCLC.

103. The method of claim 93, wherein the ADC payload is a cytotoxin.

104. The method of claim 103, wherein the cytotoxin is a topoisomerase I inhibitor.

105. The method of claim 104, wherein the topoisomerase I inhibitor is represented by the following formula: 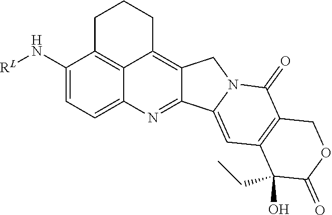
and the salts and solvates thereof, and wherein R^{L} represents a linker connected to the ADC antibody or antigen-binding fragment thereof.

106. The method of claim 93, wherein the ADC is an anti-FRα antibody or antigen-binding fragment thereof conjugated to a topoisomerase I inhibitor, wherein the topoisomerase I inhibitor is selected from:

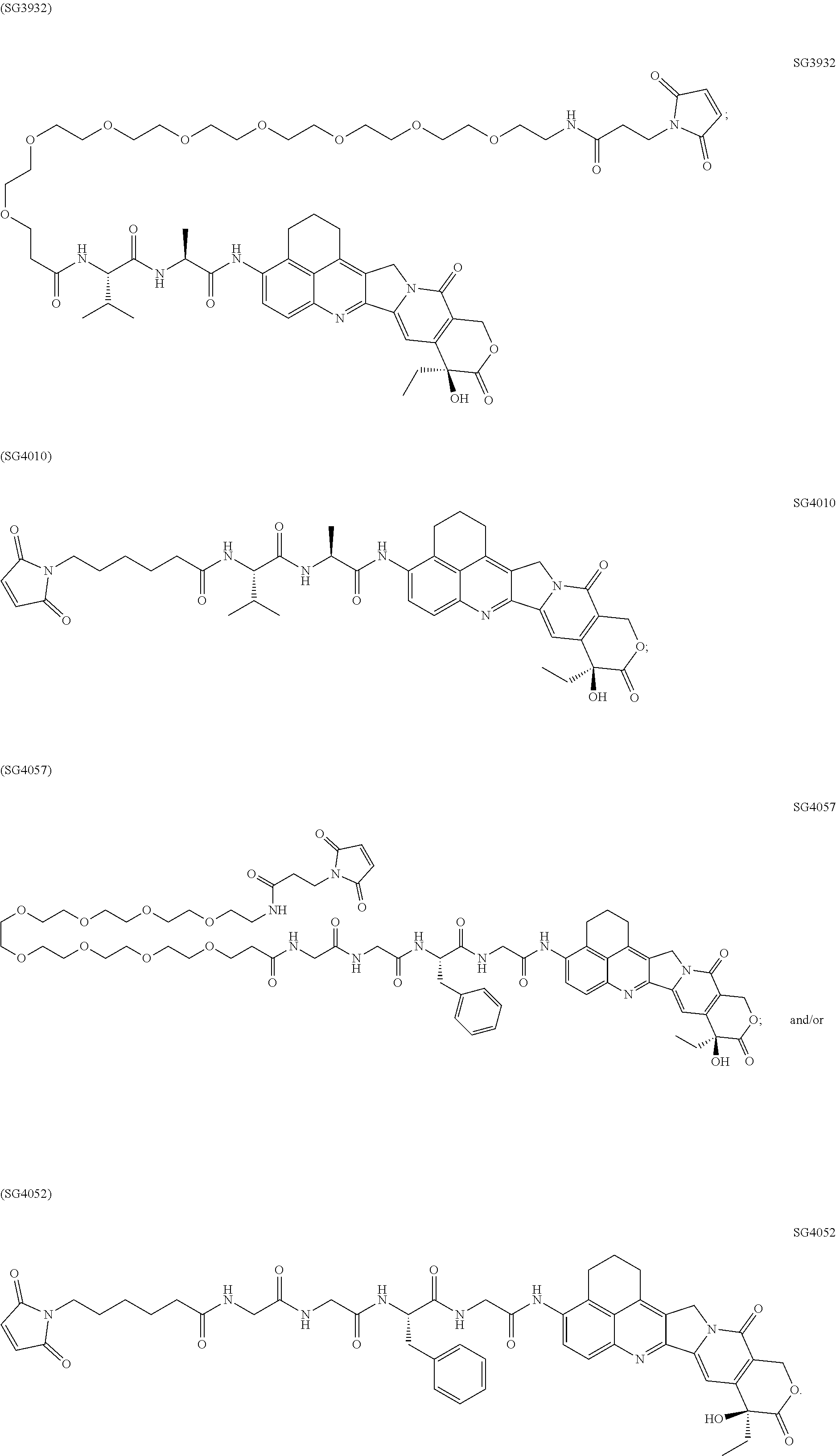

107. The method of claim 93, wherein the ADC is an anti-FRα antibody or antigen-binding fragment thereof conjugated to a topoisomerase I inhibitor, wherein the topoisomerase I inhibitor is

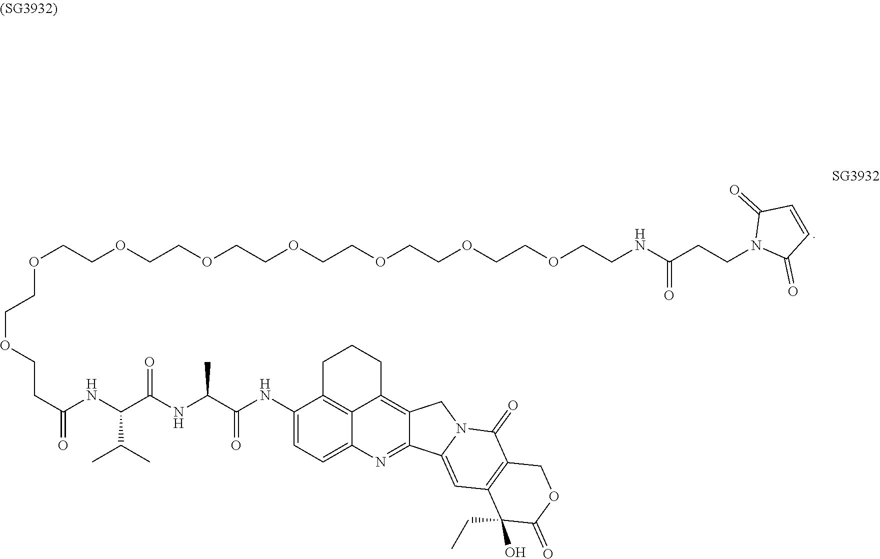 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

108. The method of claim 93, wherein the ADC has a drug-to-antibody ratio (DAR) that falls within a range selected from the group consisting of: 1 to 20, 1 to 10, 2 to 10, 2 to 8, 2 to 6, and 4 to 10.

109. The method of claim 93, wherein the ADC has a drug-to-antibody ratio (DAR) selected from the group consisting of: 4 and 8.

110. The method of claim 93, wherein the ADC antibody or antigen-binding fragment thereof includes a plurality of amino acid sequences selected from the group consisting of:
(a) a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG), a heavy chain CDR3 of SEQ ID NO: 9 (EMQQLALDY), a light chain CDR1 of SEQ ID NO: 10 (RASQGISNFLA), a light chain CDR2 of SEQ ID NO: 11 (AASSLQS), and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS), a heavy chain CDR3 of SEQ ID NO: 15 (GVGRFDS), a light chain CDR1 of SEQ ID NO: 16 (RASQSISSWLA), a light chain CDR2 of SEQ ID NO: 17 (KASSLES), and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG), a heavy chain CDR3 of SEQ ID NO: 21 (GMTTLTFDY), a light chain CDR1 of SEQ ID NO: 22 (RASQGISTFLA), a light chain CDR2 of SEQ ID NO: 23 (AASSLQS), and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG), a heavy chain CDR3 of SEQ ID NO: 27 (GMTTLTFDY), a light chain CDR1 of SEQ ID NO: 28 (RASQGISSFLA), a light chain CDR2 of SEQ ID NO: 29 (AASSLQS), and a light chain CDR3 of SEQ ID NO: 30 (QQYNSYPLT); and
(f) a heavy chain CDR1 of SEQ ID NO: 31 (SDSATWN), a heavy chain CDR2 of SEQ ID NO: 32 (RTYYRSKWYSDYAVSVKS), a heavy chain CDR3 of SEQ ID NO: 33 (GGAPFDY), a light chain CDR1 of SEQ ID NO: 34 (RASQSISSWLA), a light chain CDR2 of SEQ ID NO: 35 (KASSLES), and a light chain CDR3 of SEQ ID NO: 36 (QQYNSYSMYT).

111. The method of claim 93, wherein the ADC antibody or antigen-binding fragment thereof includes a plurality of amino acid sequences selected from the group consisting of:
(a) a VH chain comprising the amino acid sequence of SEQ ID NO: 37 and a VL chain comprising the amino acid sequence of SEQ ID NO: 38;
(b) a VH chain comprising the amino acid sequence of SEQ ID NO: 39 and a VL chain comprising the amino acid sequence of SEQ ID NO: 40;
(c) a VH chain comprising the amino acid sequence of SEQ ID NO: 41 and a VL chain comprising the amino acid sequence of SEQ ID NO: 42;
(d) a VH chain comprising the amino acid sequence of SEQ ID NO: 43 and a VL chain comprising the amino acid sequence of SEQ ID NO: 44;
(e) a VH chain comprising the amino acid sequence of SEQ ID NO: 45 and a VL chain comprising the amino acid sequence of SEQ ID NO: 46; and
(f) a VH chain comprising the amino acid sequence of SEQ ID NO: 47 and a VL chain comprising the amino acid sequence of SEQ ID NO: 48.

112. The method of claim 93, wherein the ADC antibody or antigen-binding fragment thereof includes a plurality of amino acid sequences selected from the group consisting of:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; and
(f) a heavy chain amino acid sequence of SEQ ID NO: 59 and a light chain amino acid sequence of SEQ ID NO: 60.

113. The method of claim 93,
wherein the ADC antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT),
wherein the ADC payload is a topoisomerase I inhibitor 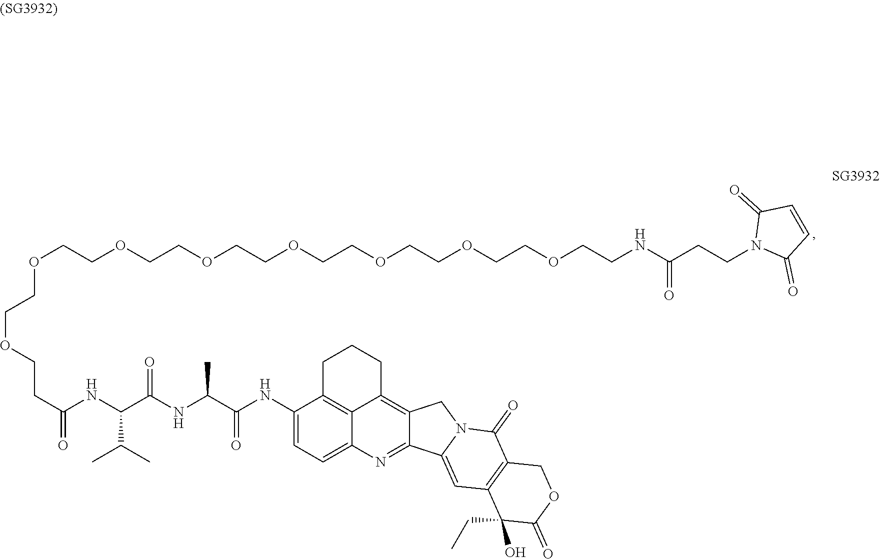 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]
and
wherein the ADC has a drug-to-antibody ratio (DAR) of 8.

114. The method of claim 113, wherein the ADC antibody or antigen-binding fragment thereof includes a VH chain comprising the amino acid sequence of SEQ ID NO: 37 and a VL chain comprising the amino acid sequence of SEQ ID NO: 38.

115. A method of generating a predicted efficacy score to predict a response of a cancer patient to an antibody drug conjugate (ADC) that includes an ADC payload and an ADC antibody that targets a protein on cancer cells, wherein the protein is folate receptor alpha (FRα), comprising:
staining a tissue sample immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell a mean optical density of staining by the dye in the membrane of the cancer cell;
determining for each cancer cell a mean optical density of staining by the dye in the cytoplasm of the cancer cell;
determining a difference between the mean optical density of staining of the membrane and the mean optical density of staining of the cytoplasm for each cancer cell in the digital image;
identifying from among all cancer cells in the digital image an 85% quantile of the difference between the mean optical density of staining of the membrane and the mean optical density of staining of the cytoplasm; and
recommending a therapy involving the ADC to the cancer patient if the 85% quantile of the difference exceeds a predetermined difference threshold.

116. The method of claim 115, further comprising:
administering the therapy involving the ADC to the cancer patient when the 85% quantile of the difference exceeds the predetermined difference threshold.

117. The method of claim 115, wherein the difference threshold is in a range of 21 to 25 on a scale of optical density having a maximum of 220.

118. The method of claim 115, wherein the detecting of cancer cells involves detecting for each cancer cell the pixels that belong to the membrane and the pixels that belong to the cytoplasm.

119. The method of claim 115, wherein the staining intensity of each membrane is computed based on an average optical density of a brown diaminobenzidine (DAB) signal in pixels of the membrane.

120. The method of claim 115, wherein the ADC antibody is a humanized IgG1 monoclonal antibody.

121. The method of claim 115, wherein the dye is 3,3′-Diaminobenzidine (DAB).

122. The method of claim 115, wherein the cancer patient has a cancer selected from the group consisting of: ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

123. The method of claim 115, wherein the cancer patient has a cancer selected from the group consisting of: ovarian cancer, non-small cell lung cancer (NSCLC) and breast cancer.

124. The method of claim 123, wherein the NSCLC is a selected from the group consisting of: squamous NSCLC, adenocarcinoma NSCLC, and a combination squamous NSCLC and adenocarcinoma NSCLC.

125. The method of claim 115, wherein the ADC payload is a cytotoxin.

126. The method of claim 125, wherein the cytotoxin is a topoisomerase I inhibitor.

127. The method of claim 126, wherein the topoisomerase I inhibitor is represented by the following formula: 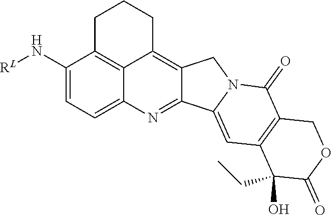
and the salts and solvates thereof, and wherein R^{L} represents a linker connected to the ADC antibody or antigen-binding fragment thereof.

128. The method of claim 115, wherein the ADC is an anti-FRα antibody or antigen-binding fragment thereof conjugated to a topoisomerase I inhibitor, wherein the topoisomerase I inhibitor is selected from:

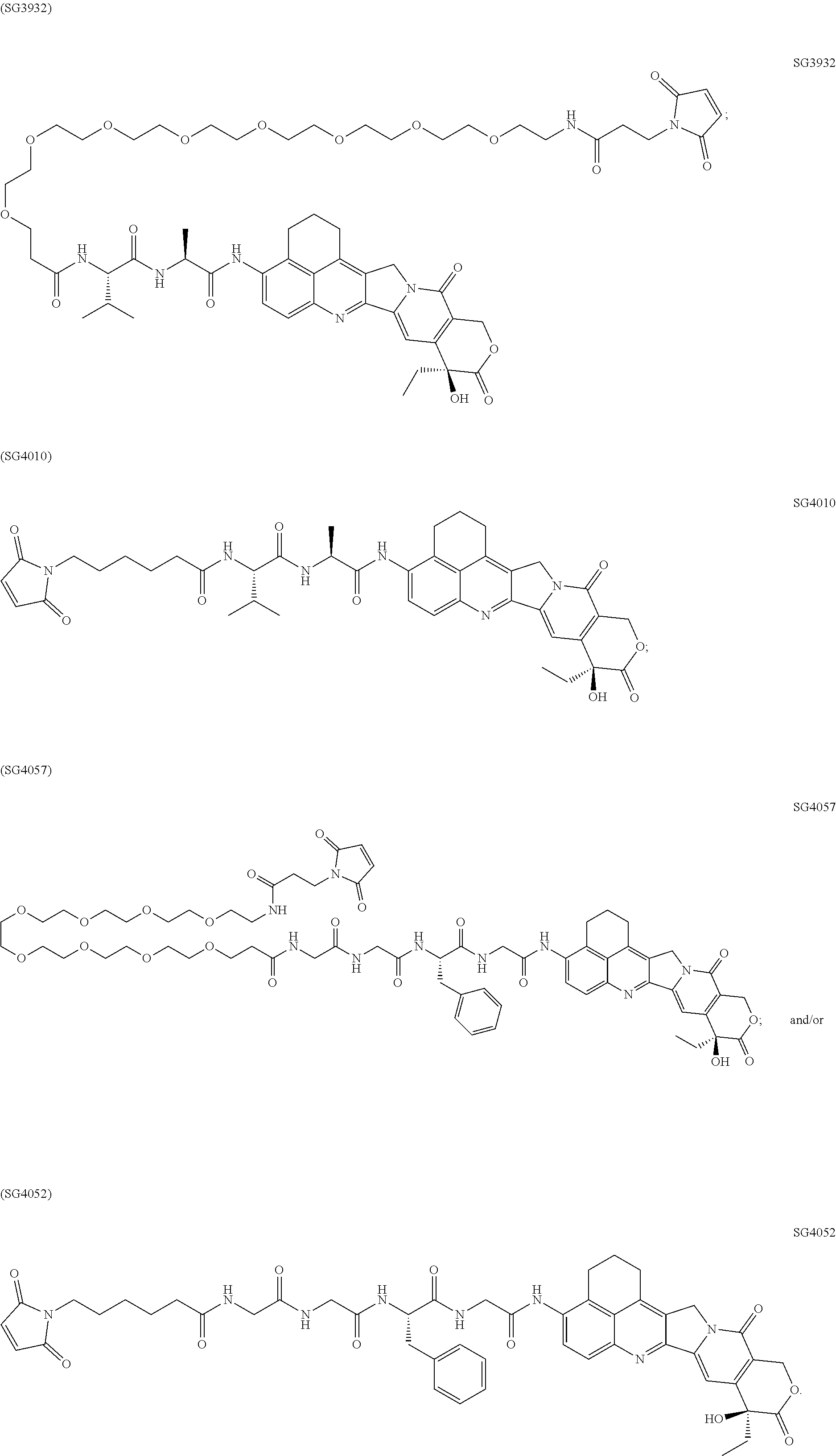

129. The method of claim 115, wherein the ADC is an anti-FRα antibody or antigen-binding fragment thereof conjugated to a topoisomerase I inhibitor, wherein the topoisomerase I inhibitor is

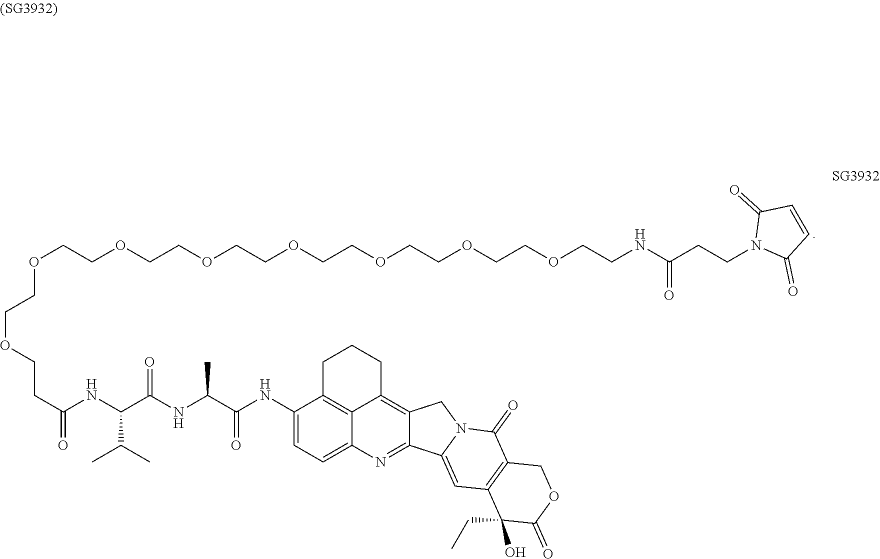 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

130. The method of claim 115, wherein the ADC has a drug-to-antibody ratio (DAR) that falls within a range selected from the group consisting of: 1 to 20, 1 to 10, 2 to 10, 2 to 8, 2 to 6, and 4 to 10.

131. The method of claim 115, wherein the ADC has a drug-to-antibody ratio (DAR) selected from the group consisting of: 4 and 8.

132. The method of claim 115, wherein the ADC antibody or antigen-binding fragment thereof includes a plurality of amino acid sequences selected from the group consisting of:
(a) a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG), a heavy chain CDR3 of SEQ ID NO: 9 (EMQQLALDY), a light chain CDR1 of SEQ ID NO: 10 (RASQGISNFLA), a light chain CDR2 of SEQ ID NO: 11 (AASSLQS), and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS), a heavy chain CDR3 of SEQ ID NO: 15 (GVGRFDS), a light chain CDR1 of SEQ ID NO: 16 (RASQSISSWLA), a light chain CDR2 of SEQ ID NO: 17 (KASSLES), and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG), a heavy chain CDR3 of SEQ ID NO: 21 (GMTTLTFDY), a light chain CDR1 of SEQ ID NO: 22 (RASQGISTFLA), a light chain CDR2 of SEQ ID NO: 23 (AASSLQS), and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG), a heavy chain CDR3 of SEQ ID NO: 27 (GMTTLTFDY), a light chain CDR1 of SEQ ID NO: 28 (RASQGISSFLA), a light chain CDR2 of SEQ ID NO: 29 (AASSLQS), and a light chain CDR3 of SEQ ID NO: 30 (QQYNSYPLT); and
(f) a heavy chain CDR1 of SEQ ID NO: 31 (SDSATWN), a heavy chain CDR2 of SEQ ID NO: 32 (RTYYRSKWYSDYAVSVKS), a heavy chain CDR3 of SEQ ID NO: 33 (GGAPFDY), a light chain CDR1 of SEQ ID NO: 34 (RASQSISSWLA), a light chain CDR2 of SEQ ID NO: 35 (KASSLES), and a light chain CDR3 of SEQ ID NO: 36 (QQYNSYSMYT).

133. The method of claim 115, wherein the ADC antibody or antigen-binding fragment thereof includes a plurality of amino acid sequences selected from the group consisting of:
(a) a VH chain comprising the amino acid sequence of SEQ ID NO: 37 and a VL chain comprising the amino acid sequence of SEQ ID NO: 38;
(b) a VH chain comprising the amino acid sequence of SEQ ID NO: 39 and a VL chain comprising the amino acid sequence of SEQ ID NO: 40;
(c) a VH chain comprising the amino acid sequence of SEQ ID NO: 41 and a VL chain comprising the amino acid sequence of SEQ ID NO: 42;
(d) a VH chain comprising the amino acid sequence of SEQ ID NO: 43 and a VL chain comprising the amino acid sequence of SEQ ID NO: 44;
(e) a VH chain comprising the amino acid sequence of SEQ ID NO: 45 and a VL chain comprising the amino acid sequence of SEQ ID NO: 46; and
(f) a VH chain comprising the amino acid sequence of SEQ ID NO: 47 and a VL chain comprising the amino acid sequence of SEQ ID NO: 48.

134. The method of claim 115, wherein the ADC antibody or antigen-binding fragment thereof includes a plurality of amino acid sequences selected from the group consisting of:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; and
(f) a heavy chain amino acid sequence of SEQ ID NO: 59 and a light chain amino acid sequence of SEQ ID NO: 60.

135. The method of claim 115,
wherein the ADC antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS), a heavy chain CDR3 of SEQ ID NO: 3 (GVGSFDY), a light chain CDR1 of SEQ ID NO: 4 (RASQSISSWLA), a light chain CDR2 of SEQ ID NO: 5 (KASGLES), and a light chain CDR3 of SEQ ID NO: 6 (QQYNSYSQLT),
wherein the ADC payload is a topoisomerase I inhibitor 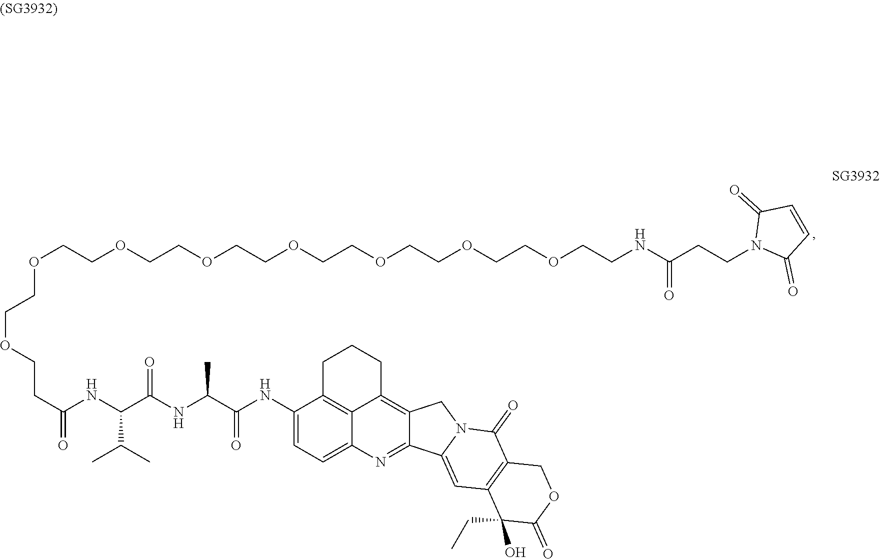 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]
and
wherein the ADC has a drug-to-antibody ratio (DAR) of 8.

136. The method of claim 135, wherein the ADC antibody or antigen-binding fragment thereof includes a VH chain comprising the amino acid sequence of SEQ ID NO: 37 and a VL chain comprising the amino acid sequence of SEQ ID NO: 38.


SEQUENCE LISTING (113 sequences)

SEQ ID NO: 1 - protein, 7 aa, synthetic construct
SDSATWN

SEQ ID NO: 2 - protein, 18 aa, synthetic construct
RTYYRSKWYNDYAVSVKS

SEQ ID NO: 3 - protein, 7 aa, synthetic construct
GVGSFDY

SEQ ID NO: 4 - protein, 11 aa, synthetic construct
RASQSISSWLA

SEQ ID NO: 5 - protein, 7 aa, synthetic construct
KASGLES

SEQ ID NO: 6 - protein, 10 aa, synthetic construct
QQYNSYSQLT

SEQ ID NO: 7 - protein, 5 aa, synthetic construct
SYAMS

SEQ ID NO: 8 - protein, 17 aa, synthetic construct
SISSGRSYIYYADSVKG

SEQ ID NO: 9 - protein, 9 aa, synthetic construct
EMQQLALDY

SEQ ID NO: 10 - protein, 11 aa, synthetic construct
RASQGISNFLA

SEQ ID NO: 11 - protein, 7 aa, synthetic construct
AASSLQS

SEQ ID NO: 12 - protein, 9 aa, synthetic construct
QQYNSYPFT

SEQ ID NO: 13 - protein, 7 aa, synthetic construct
SNSAAWN

SEQ ID NO: 14 - protein, 18 aa, synthetic construct
RTYYRSNWYNDYTLSVKS

SEQ ID NO: 15 - protein, 7 aa, synthetic construct
GVGRFDS

SEQ ID NO: 16 - protein, 11 aa, synthetic construct
RASQSISSWLA

SEQ ID NO: 17 - protein, 7 aa, synthetic construct
KASSLES

SEQ ID NO: 18 - protein, 10 aa, synthetic construct
QEYKTYSIFT

SEQ ID NO: 19 - protein, 5 aa, synthetic construct
SYNMN

SEQ ID NO: 20 - protein, 17 aa, synthetic construct
SISSGSSYIYYADSMKG

SEQ ID NO: 21 - protein, 9 aa, synthetic construct
GMTTLTFDY

SEQ ID NO: 22 - protein, 11 aa, synthetic construct
RASQGISTFLA

SEQ ID NO: 23 - protein, 7 aa, synthetic construct
AASSLQS

SEQ ID NO: 24 - protein, 9 aa, synthetic construct
QQYISYPLT

SEQ ID NO: 25 - protein, 5 aa, synthetic construct
SYSMN

SEQ ID NO: 26 - protein, 17 aa, synthetic construct
SISSRSSYVYYADSVKG

SEQ ID NO: 27 - protein, 9 aa, synthetic construct
GMTTLTFDY

SEQ ID NO: 28 - protein, 11 aa, synthetic construct
RASQGISSFLA

SEQ ID NO: 29 - protein, 7 aa, synthetic construct
AASSLQS

SEQ ID NO: 30 - protein, 9 aa, synthetic construct
QQYNSYPLT

SEQ ID NO: 31 - protein, 7 aa, synthetic construct
SDSATWN

SEQ ID NO: 32 - protein, 18 aa, synthetic construct
RTYYRSKWYSDYAVSVKS

SEQ ID NO: 33 - protein, 7 aa, synthetic construct
GGAPFDY

SEQ ID NO: 34 - protein, 11 aa, synthetic construct
RASQSISSWLA

SEQ ID NO: 35 - protein, 7 aa, synthetic construct
KASSLES

SEQ ID NO: 36 - protein, 10 aa, synthetic construct
QQYNSYSMYT

SEQ ID NO: 37 - protein, 119 aa, synthetic construct
LVQLQQSGPGLVKPSQTLSLTCAISGDSVSSDSATWNWIRQSPSRGLEWLGRTYYRSKWYNDYAVSVKSRITINPDTSKNQFSLQLNSVTPEDTAVYYCARGVGSFDYWGQGTLVTVSS

SEQ ID NO: 38 - protein, 108 aa, synthetic construct
DIQMTQSPSTLSASVGDRVTITCRASQSISSWLAWYQQKPGKAPKLLIYKASGLESGVPSRFSGSGSGTEFTLTISSLQPDDFATYYCQQYNSYSQLTFGGGTKVEIK

SEQ ID NO: 39 - protein, 118 aa, synthetic construct
EVQLVESGGGLVKPGGSLRLSCAASGFTFSSYAMSWVRQAPGKGLEWVSSISSGRSYIYYADSVKGRFTISRDNAKNSLYLKMNSLRDEDTAVYYCAREMQQLALDYWGQGTLVTVSS

SEQ ID NO: 40 - protein, 107 aa, synthetic construct
DIQMTQSPSSLSASVGDRVTITCRASQGISNFLAWFQQAPGKAPKSLIYAASSLQSGVPSKFSGSGSGTDFTLTISSLQPEDFATYYCQQYNSYPFTFGQGTKLEIK

SEQ ID NO: 41 - protein, 119 aa, synthetic construct
QVQLQQSGPGLVKPSQTLSLTCAISGDSVSSNSAAWNWIRQSPSRGLEWLGRTYYRSNWYNDYTLSVKSRITVNPDTSKNQFSLQLNSVTPEDTAVYYCVRGVGRFDSWGQGTLVTVSS

SEQ ID NO: 42 - protein, 108 aa, synthetic construct
DIQMTQSPSTLSASVGDRVIITCRASQSISSWLAWYQQKPGKAPKLLIYKASSLESGVPSRFSGSGSGTEFTLTITSLQPDDFASYYCQEYKTYSIFTFGPGTKVDIK

SEQ ID NO: 43 - protein, 118 aa, synthetic construct
EVQLVESGGGLVKPGGSLRLSCAASGFTFSSYNMNWVRQAPGKGLEWVSSISSGSSYIYYADSMKGRFTISRDNAKNSLFLQMNSLRAEDTAVYYCARGMTTLTFDYWGQGTLVTVSS

SEQ ID NO: 44 - protein, 107 aa, synthetic construct
DIQMTQSPSSLSASVGDRVTITCRASQGISTFLAWFQQKPGKAPKSLIYAASSLQSGVPSKFSGSGSETDFTLTISSLQPEDFATYYCQQYISYPLTFGGGTKVEIK

SEQ ID NO: 45 - protein, 118 aa, synthetic construct
EVQLVESGGGLVKPGGSLRLSCAASGFTFSSYSMNWVRQAPGKGLEWVSSISSRSSYVYYADSVKGRFTISRDNAKNSLYLQMNSLRAEDTAVYYCARGMTTLTFDYWGQGTLVTVSS

SEQ ID NO: 46 - protein, 107 aa, synthetic construct
DIQMTQSPSSLSASVGDRVTITCRASQGISSFLAWFQQKPGKAPKSLIYAASSLQSGVPSKFSGSGSGTDFTLTISSLQPEDFATYYCQQYNSYPLTFGGGTKVEIK

SEQ ID NO: 47 - protein, 119 aa, synthetic construct
QVQLQQSGPGLVKPSQTLSLTCAISGDSVSSDSATWNWIRQSPSRGLEWLGRTYYRSKWYSDYAVSVKSRITINPDTSKNQFSLQLNSVTPEDTAVYFCARGGAPFDYWGQGTLVTVSS

SEQ ID NO: 48 - protein, 108 aa, synthetic construct
DIQMTQSPSTLSASVGDRVTINCRASQSISSWLAWYQQKPGKAPNLLIYKASSLESGVPSRFSGSGSGTEFTLTISSLQPDDFATYYCQQYNSYSMYTFGQGTKLEIK

SEQ ID NO: 49 - protein, 449 aa, synthetic construct
LVQLQQSGPGLVKPSQTLSLTCAISGDSVSSDSATWNWIRQSPSRGLEWLGRTYYRSKWYNDYAVSVKSRITINPDTSKNQFSLQLNSVTPEDTAVYYCARGVGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 215 aa, synthetic construct
DIQMTQSPSTLSASVGDRVTITCRASQSISSWLAWYQQKPGKAPKLLIYKASGLESGVPSRFSGSGSGTEFTLTISSLQPDDFATYYCQQYNSYSQLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 448 aa, synthetic construct
EVQLVESGGGLVKPGGSLRLSCAASGFTFSSYAMSWVRQAPGKGLEWVSSISSGRSYIYYADSVKGRFTISRDNAKNSLYLKMNSLRDEDTAVYYCAREMQQLALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 214 aa, synthetic construct
DIQMTQSPSSLSASVGDRVTITCRASQGISNFLAWFQQAPGKAPKSLIYAASSLQSGVPSKFSGSGSGTDFTLTISSLQPEDFATYYCQQYNSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 449 aa, synthetic construct
QVQLQQSGPGLVKPSQTLSLTCAISGDSVSSNSAAWNWIRQSPSRGLEWLGRTYYRSNWYNDYTLSVKSRITVNPDTSKNQFSLQLNSVTPEDTAVYYCVRGVGRFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 215 aa, synthetic construct
DIQMTQSPSTLSASVGDRVIITCRASQSISSWLAWYQQKPGKAPKLLIYKASSLESGVPSRFSGSGSGTEFTLTITSLQPDDFASYYCQEYKTYSIFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 448 aa, synthetic construct
EVQLVESGGGLVKPGGSLRLSCAASGFTFSSYNMNWVRQAPGKGLEWVSSISSGSSYIYYADSMKGRFTISRDNAKNSLFLQMNSLRAEDTAVYYCARGMTTLTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, synthetic construct
DIQMTQSPSSLSASVGDRVTITCRASQGISTFLAWFQQKPGKAPKSLIYAASSLQSGVPSKFSGSGSETDFTLTISSLQPEDFATYYCQQYISYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 448 aa, synthetic construct
EVQLVESGGGLVKPGGSLRLSCAASGFTFSSYSMNWVRQAPGKGLEWVSSISSRSSYVYYADSVKGRFTISRDNAKNSLYLQMNSLRAEDTAVYYCARGMTTLTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
DIQMTQSPSSLSASVGDRVTITCRASQGISSFLAWFQQKPGKAPKSLIYAASSLQSGVPSKFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 449 aa, synthetic construct
QVQLQQSGPGLVKPSQTLSLTCAISGDSVSSDSATWNWIRQSPSRGLEWLGRTYYRSKWYSDYAVSVKSRITINPDTSKNQFSLQLNSVTPEDTAVYFCARGGAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 215 aa, synthetic construct
DIQMTQSPSTLSASVGDRVTINCRASQSISSWLAWYQQKPGKAPNLLIYKASSLESGVPSRFSGSGSGTEFTLTISSLQPDDFATYYCQQYNSYSMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 30 aa, synthetic construct
LVQLQQSGPGLVKPSQTLSLTCAISGDSVS

SEQ ID NO: 62 - protein, 14 aa, synthetic construct
WIRQSPSRGLEWLG

SEQ ID NO: 63 - protein, 32 aa, synthetic construct
RITINPDTSKNQFSLQLNSVTPEDTAVYYCAR

SEQ ID NO: 64 - protein, 11 aa, synthetic construct
WGQGTLVTVSS

SEQ ID NO: 65 - protein, 23 aa, synthetic construct
DIQMTQSPSTLSASVGDRVTITC

SEQ ID NO: 66 - protein, 15 aa, synthetic construct
WYQQKPGKAPKLLIY

SEQ ID NO: 67 - protein, 32 aa, synthetic construct
GVPSRFSGSGSGTEFTLTISSLQPDDFATYYC

SEQ ID NO: 68 - protein, 10 aa, synthetic construct
FGGGTKVEIK

SEQ ID NO: 69 - protein, 30 aa, synthetic construct
EVQLVESGGGLVKPGGSLRLSCAASGFTFS

SEQ ID NO: 70 - protein, 14 aa, synthetic construct
WVRQAPGKGLEWVS

SEQ ID NO: 71 - protein, 32 aa, synthetic construct
RFTISRDNAKNSLYLKMNSLRDEDTAVYYCAR

SEQ ID NO: 72 - protein, 11 aa, synthetic construct
WGQGTLVTVSS

SEQ ID NO: 73 - protein, 23 aa, synthetic construct
DIQMTQSPSSLSASVGDRVTITC

SEQ ID NO: 74 - protein, 15 aa, synthetic construct
WFQQAPGKAPKSLIY

SEQ ID NO: 75 - protein, 32 aa, synthetic construct
GVPSKFSGSGSGTDFTLTISSLQPEDFATYYC

SEQ ID NO: 76 - protein, 10 aa, synthetic construct
FGQGTKLEIK

SEQ ID NO: 77 - protein, 30 aa, synthetic construct
QVQLQQSGPGLVKPSQTLSLTCAISGDSVS

SEQ ID NO: 78 - protein, 14 aa, synthetic construct
WIRQSPSRGLEWLG

SEQ ID NO: 79 - protein, 32 aa, synthetic construct
RITVNPDTSKNQFSLQLNSVTPEDTAVYYCVR

SEQ ID NO: 80 - protein, 11 aa, synthetic construct
WGQGTLVTVSS

SEQ ID NO: 81 - protein, 23 aa, synthetic construct
DIQMTQSPSTLSASVGDRVIITC

SEQ ID NO: 82 - protein, 15 aa, synthetic construct
WYQQKPGKAPKLLIY

SEQ ID NO: 83 - protein, 32 aa, synthetic construct
GVPSRFSGSGSGTEFTLTITSLQPDDFASYYC

SEQ ID NO: 84 - protein, 10 aa, synthetic construct
FGPGTKVDIK

SEQ ID NO: 85 - protein, 30 aa, synthetic construct; identical to SEQ ID NO: 69

SEQ ID NO: 86 - protein, 14 aa, synthetic construct
WVRQAPGKGLEWVS

SEQ ID NO: 87 - protein, 32 aa, synthetic construct
RFTISRDNAKNSLFLQMNSLRAEDTAVYYCAR

SEQ ID NO: 88 - protein, 11 aa, synthetic construct
WGQGTLVTVSS

SEQ ID NO: 89 - protein, 23 aa, synthetic construct
DIQMTQSPSSLSASVGDRVTITC

SEQ ID NO: 90 - protein, 15 aa, synthetic construct
WFQQKPGKAPKSLIY

SEQ ID NO: 91 - protein, 32 aa, synthetic construct
GVPSKFSGSGSETDFTLTISSLQPEDFATYYC

SEQ ID NO: 92 - protein, 10 aa, synthetic construct
FGGGTKVEIK

SEQ ID NO: 93 - protein, 30 aa, synthetic construct; identical to SEQ ID NO: 69

SEQ ID NO: 94 - protein, 14 aa, synthetic construct
WVRQAPGKGLEWVS

SEQ ID NO: 95 - protein, 32 aa, synthetic construct
RFTISRDNAKNSLYLQMNSLRAEDTAVYYCAR

SEQ ID NO: 96 - protein, 11 aa, synthetic construct
WGQGTLVTVSS

SEQ ID NO: 97 - protein, 23 aa, synthetic construct
DIQMTQSPSSLSASVGDRVTITC

SEQ ID NO: 98 - protein, 15 aa, synthetic construct
WFQQKPGKAPKSLIY

SEQ ID NO: 99 - protein, 32 aa, synthetic construct; identical to SEQ ID NO: 75

SEQ ID NO: 100 - protein, 10 aa, synthetic construct
FGGGTKVEIK

SEQ ID NO: 101 - protein, 30 aa, synthetic construct; identical to SEQ ID NO: 77

SEQ ID NO: 102 - protein, 14 aa, synthetic construct
WIRQSPSRGLEWLG

SEQ ID NO: 103 - protein, 32 aa, synthetic construct
RITINPDTSKNQFSLQLNSVTPEDTAVYFCAR

SEQ ID NO: 104 - protein, 11 aa, synthetic construct
WGQGTLVTVSS

SEQ ID NO: 105 - protein, 23 aa, synthetic construct
DIQMTQSPSTLSASVGDRVTINC

SEQ ID NO: 106 - protein, 15 aa, synthetic construct
WYQQKPGKAPNLLIY

SEQ ID NO: 107 - protein, 32 aa, synthetic construct; identical to SEQ ID NO: 67

SEQ ID NO: 108 - protein, 10 aa, synthetic construct
FGQGTKLEIK

SEQ ID NO: 109 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 107 aa, synthetic construct [sequence omitted]

SEQ ID NO: 111 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 112 - protein, 210 aa, Homo sapiens
RIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMS

SEQ ID NO: 113 - protein, 210 aa, Macaca fascicularis
RTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMS